(19) United States
(12) Patent Application Publication
(10) Pub. No.: US 2006/0280789 A1
(43) Pub. Date: Dec. 14, 2006

(54) Sustained release formulations

(75) Inventors: Yosuke Ueki, Kakamigahara-shi (JP); Satoshi Fujioka, Kakamigahara-shi (JP); Shigeru Aoki, Kakamigahara-shi (JP); Susan Abu-Shakra, Malvern, PA (US); J. Paul Carter, North Andover, MA (US); Gary Dorough, Andrews AFB, MD (US)

Correspondence Address: VENABLE LLP, P.O. BOX 34385, WASHINGTON, DC, 20043-9998, US

(73) Assignee: Eisai Research Institute, Andover, MA (US)

(21) Appl. No.: 11/317,897

(22) Filed: Dec. 27, 2005

Related U.S. Application Data
(60) Provisional application No. 60/663,723, filed on Mar. 22, 2005.
(60) Provisional application No. 60/675,482, filed on Apr. 28, 2005.

(30) Foreign Application Priority Data
Apr. 28, 2005 (JP) 2005-132338
Apr. 6, 2005 (JP) 2005-110404
Dec. 27, 2004 (JP) 2004-376770

(51) Int. Cl. A61K 31/445; A61K 9/20

(52) U.S. Cl. 424/464; 514/319

(57) ABSTRACT

The invention provides sustained release formulations of basic drugs, stereoisomers of basic drugs, pharmaceutically acceptable salts of basic drugs, and pharmaceutically acceptable salts of stereoisomers of basic drugs. The basic drugs may be anti-dementia drugs, such as cholinesterase inhibitors or memantine. In one embodiment, the cholinesterase inhibitor is donepezil.

### RELATED APPLICATIONS

This application claims priority to U.S. Provisional Application No. 60/675,482 filed Apr. 28, 2005; Japanese Priority Patent Application No. 2005-132338 filed Apr. 28, 2005; Japanese Priority Patent Application No. 2005-110404 filed Apr. 6, 2005; U.S. Provisional Application No. 60/663,723 filed Mar. 22, 2005; and Japanese Priority Patent Application No. 2004-376770 filed Dec. 27, 2004; the disclosures of which are incorporated by reference herein in their entirety.

### FIELD OF THE INVENTION

The invention provides sustained release formulations of basic drugs, stereoisomers of basic drugs, pharmaceutically acceptable salts of basic drugs, or pharmaceutically acceptable salts of stereoisomers of basic drugs. The basic drugs may be any known in the art, such as anti-dementia drugs. In one embodiment, the anti-dementia drug is a cholinesterase inhibitor.

### BACKGROUND OF THE INVENTION

Cholinesterase inhibitors, such as donepezil hydrochloride, are currently available in oral dosage forms (e.g., ARICEPT®, Eisai, Inc., Teaneck, N.J.) that provide for immediate release of the cholinesterase inhibitor to treat, for example, Alzheimer's disease. ARICEPT® is available in 5 mg and 10 mg oral dosage forms and is generally administered once per day.

The immediate release of cholinesterase inhibitors, such as ARICEPT®, results in a spike in the patient's blood plasma levels within 2 to 5 hours after administration of the drug. FIG. 10, taken from Rogers et al, Br. J Clin. Pharmacol., 46(Suppl. 1):1-6 (1998), shows the mean plasma concentration time curves following single dose administrations of 2.0 mg, 4.0 mg and 6.0 mg donepezil hydrochloride to groups of six healthy male volunteers. As can be seen from FIG. 10, the patients administered 2.0 mg donepezil hydrochloride experienced a peak plasma concentration (Cₘₐₓ) of 3.2±0.6 ng/ml and the time at which the peak concentration occurred (tₘₐₓ) was 4.5±1.2 hours; the patients administered 4.0 mg donepezil hydrochloride experienced a Cₘₐₓ of 6.9±0.7 ng/ml at a tₘₐₓ of 4.7±1.9 hours; and the patients administered 6.0 mg donepezil hydrochloride experienced a Cₘₐₓ, of 11.6±2.8 ng/ml at a tₘₐₓ of 3.2±1.5 hours. The total area under the plasma concentration-time curve (AUC_{(t-∞)}) for patients administered 2.0 mg, 4.0 mg and 6.0 mg donepezil hydrochloride was 225.1±82.6 ng·h/ml; 518.6±154.5 ng·h/ml; and 706.6±195.8ng·h/ml, respectively.

FIG. 11, taken from Yasui-Furukori et al, Journal of Chromatography B, 768:261-265 (2002), shows the plasma concentration versus time curves of donepezil hydrochloride after a single oral does of 5 mg was given to two volunteers. The first volunteer experienced a Cₘₐₓ of 17.6 ng/ml at a tₘₐₓ of 4 hours, while the second volunteer experienced a Cₘₐₓ of 13.7 ng/ml at a tₘₐₓ of 2 hours. The AUC_{(t-∞)} for the two volunteers was 628 ng·h/ml and 416 ng·h/ml, respectively.

FIG. 12, taken from Tiseo et al, Br. J Clin. Pharmacol., 46(Suppl. 1):13-18 (1998), shows the mean plasma concentration-time curves for 5 mg and 10 mg donepezil over the course of a 37 day study, where the full pharmacokinetic profiles were undertaken on days 1, 7, 14, 21 and 28, and all other time-points represent the trough levels. The pharmacokinetic parameters of donepezil at steady state shown in FIG. 12 are numerically shown in Table A below.

TABLE A

| Parameter | Donepezil 5 mg | Donepezil 10 mg |
|---|---|---|
| Cₘᵢₙ (ng/ml) | 24.1 ± 3.8 | 38.5 ± 8.6 |
| Cₘₐₓ (ng/ml) | 34.1 ± 7.3 | 60.5 ± 10.0 |
| tₘₐₓ (h) | 3.0 ± 1.4 | 3.9 ± 1.0 |
| Cₛₛ (ng/ml) | 26.4 ± 3.9 | 47.0 ± 8.0 |
| AUC₍₀₋₂₄₎ (ng · h/ml) | 634.8 ± 92.2 | 1127.8 ± 195.9 |
| AUC_{(0-∞)} (ng · h/ml) | 2889.3 ± 751.6 | 5051.9 ± 1613.6 |

The initial spike in blood plasma levels at tₘₐₓ may cause undesirable side effects in patients, such as anxiety, nightmares, insomnia, and/or gastrointestinal problems (e.g., nausea, emesis, diarrhea).

### SUMMARY OF THE INVENTION

Compared to immediate release formulations, a sustained release formulation containing a physiologically active drug allows blood concentrations of the drug to be maintained for a long time or above the therapeutic concentration. Accordingly, by achieving the sustained-release characteristics of a drug it may be possible to reduce the number of dosings while providing the same or better therapeutic effects—potentially improving compliance. With the sustained-release characteristics of the drug, it may also be possible to avoid a rapid increase in blood plasma concentration levels immediately after administration of the drug, thus potentially reducing or eliminating adverse side effects. There is a need in the art for new drug formulations to treat Alzheimer's disease that overcome the side effects of immediate release formulations or that provide other benefits over immediate release formulations. The invention is directed to these, as well as other, important ends.

The invention provides sustained release formulations of basic (alkaline) drugs, such as cholinesterase inhibitors. The term “basic drugs” includes basic drugs, stereoisomers of basic drugs, pharmaceutically acceptable salts of basic drugs, and pharmaceutically acceptable salts of stereoisomers of basic drugs. In one embodiment, the basic drug is a cholinesterase inhibitor. The term “cholinesterase inhibitor” includes cholinesterase inhibitors, stereoisomers of cholinesterase inhibitors, pharmaceutically acceptable salts of cholinesterase inhibitors, and pharmaceutically acceptable salts of stereoisomers of cholinesterase inhibitors. In one embodiment, the cholinesterase inhibitor is donepezil, a stereoisomer thereof and/or a pharmaceutically acceptable salt thereof. The phrase “donepezil, a pharmaceutically acceptable salt thereof, and/or a stereoisomer thereof” refers to donepezil, pharmaceutically acceptable salts of donepezil, stereoisomers of donepezil, and pharmaceutically acceptable salts of stereoisomers of donepezil. When used in the context of a dosage amount, such as “between 1 and 10 milligrams of donepezil or a pharmaceutically acceptable salt thereof,” the numerical weight refers to the weight of donepezil, exclusive of any salt, counterion, and so on. Therefore, to obtain the equivalent of 10 milligrams of donepezil, it would be necessary to use more than 10 milligrams of donepezil hydrochloride, due to the additional weight of the hydrochloride.

The term “patient” refers to mammals, preferably humans. The term “patient” includes males and females, and includes neonates, children and adults.

In one embodiment, the invention provides orally administrable formulations comprising from 1 milligram to 50 milligrams donepezil or a pharmaceutically acceptable salt thereof; wherein the formulations provide blood plasma concentration levels in a patient wherein the ratio of the maximum steady state plasma concentration to the minimum steady state plasma concentration is from 1.00 to 1.50; and wherein the steady state plasma concentration is measured after a patient is administered the formulation on a daily basis for at least three weeks. The blood samples are taken at intervals beginning immediately after the last dose is taken and are taken at intervals for a period of between 3 and 5 half-lives of the drug.

In other embodiments, the invention provides orally administrable formulations comprising from 10 milligrams to 25 milligrams donepezil or a pharmaceutically acceptable salt thereof; wherein the formulations provide blood plasma concentration levels in a patient wherein the maximum steady state plasma concentration is 5% to 15% higher than the average steady state plasma concentration; and wherein the steady state plasma concentration is measured after a patient is administered the formulation on a daily basis for at least three weeks.

In another embodiment, the invention provides orally administrable formulations comprising from 10 milligrams to 25 milligrams donepezil or a pharmaceutically acceptable salt thereof; wherein the formulations provide steady state plasma concentrations in a patient from 2.0 to 3.0 ng/ml per milligram of donepezil or the pharmaceutically acceptable salt thereof in the formulation; and wherein the steady state plasma concentration is measured after a patient is administered the formulation on a daily basis for at least three weeks.

In another embodiment, the invention provides orally administrable formulations comprising from 1 milligram to 50 milligrams donepezil or a pharmaceutically acceptable salt thereof; wherein the formulations provide blood plasma concentration levels in a patient wherein the maximum steady state plasma concentration is 1% to 40% higher than the minimum steady state plasma concentration; and wherein the steady state plasma concentration is measured after a patient is administered the formulation on a daily basis for at least three weeks.

In another embodiment, the invention provides orally administrable formulations comprising from 1 milligram to 50 milligrams donepezil or a pharmaceutically acceptable salt thereof; wherein the formulations provide blood plasma concentration levels in a patient having maximum steady state plasma concentrations that are 5% to 25% higher than the average steady state plasma concentrations; and wherein the steady state plasma concentration is measured after a patient is administered the formulation on a daily basis for at least three weeks.

In certain embodiments, the invention provides orally administrable sustained release formulations comprising an amount of donepezil (or a donepezil salt) selected from 9 milligrams to 30 milligrams; from 10 milligrams to 25 milligrams; from 14 milligrams to 23 milligrams donepezil; from 13 to 15 milligrams; from 22 to 24 milligrams; from 11 milligrams to 17.5 milligrams; or from 18 milligrams to 29 milligrams. In other embodiments, the invention provides orally administrable sustained release formulations comprising 14 milligrams, 15 milligrams, 20 milligrams, or 23 milligrams of donepezil (or a donepezil salt). In other embodiments, the invention provides an orally administrable sustained release formulations consisting essentially of 14 milligrams, 15 milligrams, 20 milligrams, or 23 milligrams donepezil (or a donepezil salt), respectively. The sustained release formulations may release donepezil with a maximum concentration of release occurring between 8 and 12 hours after administration of the formulation to the patient; or 10 hours after administration. The donepezil may be in the form of a pharmaceutically acceptable salt and/or a stereoisomer.

In one embodiment, the invention provides orally administrable sustained release formulations comprising 10 milligrams to 25 milligrams donepezil that provide a steady state serum concentration in a patient of 45 to 55 ng/ml; from 48 to 53 ng/ml; from 50 to 52 ng/ml; or 51 ng/ml. The sustained release formulations may comprise 14 milligrams, 15 milligrams, 20 milligrams or 23 milligrams donepezil. The donepezil may be in the form of a pharmaceutically acceptable salt and/or a stereoisomer.

In one embodiment, the invention provides orally administrable sustained release formulations comprising from 10 milligrams to 25 milligrams donepezil that provide serum concentrations of donepezil in a patient wherein the ratio of the maximum steady state serum concentration to the minimum steady state serum concentration is from 1.00 to 1.50; from 1.05 to 1.4; from 1.1 to 1.3; or 1.2. The sustained release formulations may comprise 14 milligrams, 15 milligrams, 20 milligrams, or 23 milligrams donepezil. The donepezil may be in the form of a pharmaceutically acceptable salt and/or a stereoisomer.

In one embodiment, the invention provides orally administrable sustained release formulations comprising 10 milligrams to 25 milligrams donepezil that provide serum concentrations of donepezil in a patient wherein the ratio of the maximum steady state serum concentration to the average steady state serum concentration is from 1.0 to 1.2; from 1.05 to 1.15; or 1.1. The sustained release formulations may comprise 14 milligrams, 15 milligrams, 20 milligrams, or 23 milligrams donepezil. The donepezil may be in the form of a pharmaceutically acceptable salt and/or a stereoisomer.

In one embodiment, the invention provides orally administrable sustained release formulations comprising 10 milligrams to 25 milligrams donepezil that provide serum concentrations of donepezil in a patient wherein the area under the curve (AUC) is from 1100 to 1300 ng/ml/hour; from 1150 to 1250 ng/ml/hour; or 1200 ng/ml/hour. The sustained release formulations may comprise 14 milligrams, 15 milligrams, 20 milligrams, or 23 milligrams donepezil. The donepezil may be in the form of a pharmaceutically acceptable salt and/or a stereoisomer.

In another embodiment, the invention provides orally administrable sustained release formulations comprising a basic drug, an enteric polymer, and, optionally, one or more compounds selected from water-insoluble polymers, water-soluble sugars, sugar alcohols, and pharmaceutically acceptable excipients. In another embodiment, the invention provides orally administrable sustained release formulations comprising (i) from 1 to 50% by weight of at least one cholinesterase inhibitor; (ii) from 5 to 90% by weight of at least one enteric polymer; (iii) from 1 to 75% by weight of at least one water-insoluble polymer; (iv) from 10 to 70% by weight of one or more (a) water soluble sugars, (b) water soluble sugar alcohols, or (c) water soluble sugars and water soluble sugar alcohols; and (v) optionally one or more other pharmaceutically acceptable excipients. In one embodiment, the pharmaceutically acceptable excipients may comprise from 0 to 5.0% by weight, or from 0.01% to 5.0% by weight, of at least one lubricant and/or from 0 to 5.0% by weight, or from 0.01% to 5.0% by weight, of at least one binder. The term “% by weight” is percentage by weight based on the total weight of the formulation.

In another embodiment, the invention provides orally administrable sustained release pharmaceutical formulations comprising: (i) from 2.5% to 20.0% by weight donepezil or a pharmaceutically acceptable salt thereof; (ii) from 5.0% to 30.0% by weight of at least one enteric polymer; (iii) from 20.0% to 35.0% by weight of at least one water-insoluble polymer; (iv) from 35.0% to 55.0% by weight of (a) at least one water-soluble sugar, (b) at least one water-soluble sugar alcohol, or (c) at least one water-soluble sugar and water-soluble sugar alcohol; and (v) from 0 to 10.0% by weight of one or more pharmaceutically acceptable excipients.

In another embodiment, the invention provides orally administrable sustained release pharmaceutical formulations comprising: (i) from 5.0% to 15.0% by weight donepezil or a pharmaceutically acceptable salt thereof; (ii) from 10.0% to 25.0% by weight of at least one methacrylic acid copolymer; (iii) from 20.0% to 30.0% by weight of at least one C₁₋₆ alkyl cellulose; (iv) from 40.0% to 50.0% by weight of at least one compound selected from the group consisting of lactose, sucrose, glucose, dextrin, pullulan, mannitol, erythritol, xylitol and sorbitol; and (v) from 0.01 to 5.0% by weight of one or more pharmaceutically acceptable excipients.

In another embodiment, the invention provides orally administrable sustained release pharmaceutical formulations comprising from 5.0% to 13.0% by weight donepezil or a pharmaceutically acceptable salt thereof; from 40.0% to 50.0% by weight lactose; from 20.0% to 30.0% by weight ethylcellulose; from 10.0% to 20.0% by weight of a methacrylic acid-methylmethacrylate copolymer; and from 0.1% to 5.0% by weight of one or more pharmaceutically acceptable excipients. In one embodiment, the invention provides sustained release pharmaceutical formulations comprising from 6.5% to 7.5% by weight donepezil or a pharmaceutically acceptable salt thereof. In one embodiment, the invention provides sustained release pharmaceutical formulations comprising from 7.0% to 8.0% by weight donepezil or a pharmaceutically acceptable salt thereof. In one embodiment, the invention provides sustained release pharmaceutical formulations comprising from 9.5% to 10.5% by weight donepezil or a pharmaceutically acceptable salt thereof. In another embodiment, the invention provides sustained release pharmaceutical formulations comprising from 11.0% to 12.0% by weight donepezil or a pharmaceutically acceptable salt thereof.

In other embodiments, the invention provides sustained release pharmaceutical formulations comprising from 7.0% to 8.0% by weight donepezil or a pharmaceutically acceptable salt thereof; from 46.2% to 47.2% by weight lactose; from 26.5% to 27.5% by weight ethylcellulose; from 15.5% to 16.5% by weight of a methacrylic acid-methylmethacrylate copolymer; from 2% to 3% by weight hydroxypropyl cellulose, and from 0.1% to 0.5% by weight magnesium stearate. In other embodiments, the invention provides formulations comprising from 9.5% to 10.5% by weight donepezil or a pharmaceutically acceptable salt thereof; from 43.2% to 44.2% by weight lactose; from 24.5% to 25.5% by weight ethylcellulose; from 17.5% to 18.5% by weight of a methacrylic acid-methylmethacrylate copolymer; from 2.5% to 3.5% by weight hydroxypropyl cellulose; and from 0.1% to 0.5% by weight magnesium stearate.

### BRIEF DESCRIPTION OF THE FIGURES

FIG. 1 shows the dissolution profile of donepezil hydrochloride in a 0.1 N hydrochloric acid solution in the matrix sustained release formulations of Examples 2 and 4, where Comparative Example 1 is the control experiment.

FIG. 2 shows the dissolution profile of donepezil hydrochloride in a 50 mM phosphoric acid buffer (pH 6.8) in the matrix sustained release formulations of Examples 2 and 4, where Comparative Example 1 is the control experiment.

FIG. 3 shows the dissolution profile of donepezil hydrochloride in a 0.1 N hydrochloric acid solution in the matrix sustained release formulations of Examples 14-17.

FIG. 4 shows the dissolution profile of donepezil hydrochloride in a 50 mM phosphoric acid buffer (pH 6.8) in the matrix sustained release formulations of Examples 14-17.

FIG. 5 shows the dissolution profile of donepezil hydrochloride in a 0.1 N hydrochloric acid solution in the matrix sustained release formulations of Examples 12 and 13, where Comparative Example 2 is the control experiment.

FIG. 6 shows the dissolution profile of donepezil hydrochloride in a 50 mM phosphoric acid buffer (pH 6.8) in the matrix sustained release formulations of Examples 12 and 13, where Comparative Example 2 is the control experiment.

FIG. 7 shows the results of dissolution profiles of donepezil hydrochloride in test solution A and test solution B in the matrix sustained release formulations of inventive Example 27.

FIG. 8 shows the results of dissolution profiles of donepezil hydrochloride in test solution A and test solution B in the matrix sustained release formulations of inventive Examples 28 and 29.

FIG. 9 shows the results of dissolution profiles of donepezil hydrochloride in test solution A and test solution B in the matrix sustained release formulations of inventive Examples 30 and 31.

FIG. 10 was taken from Rogers et al, Br. J Clin. Pharmacol., 46(Suppl. 1):1-6 (1998) and shows the mean plasma concentration time curves following single dose administrations of 2.0 mg, 4.0 mg and 6.0 mg immediate release donepezil hydrochloride to groups of six healthy male volunteers.

FIG. 11 was taken from Yasui-Furukori et al, Journal of Chromatography B, 768:261-265 (2002), and shows the plasma concentration versus time curves of donepezil hydrochloride after a single oral does of 5 mg was given to two volunteers.

FIG. 12 was taken from Tiseo et al, Br. J Clin. Pharmacol., 46(Suppl. 1):13-18 (1998), and shows the mean plasma concentration-time curves for 5 mg and 10 mg donepezil over the course of a 37 day study, where the full pharmacokinetic profiles were undertaken on days 1, 7, 14, 21 and 28, and all other time-points represent the trough levels.

### DETAILED DESCRIPTION OF THE INVENTION

The invention provides sustained release formulations of basic drugs (such as cholinesterase inhibitors) that overcome the problems associated with immediate release formulations because the basic drug is released without causing an undesirable spike (Cₘₐₓ) at tₘₐₓ, as opposed to the conventional formulations which provide for immediate release, and a consequent blood plasma spike (Cₘₐₓ), of the basic drug at tₘₐₓ. The term “sustained-release” includes “controlled-release” and “extended-release.”

In one embodiment, the invention provides sustained-release formulations comprising at least one cholinesterase inhibitor, where the difference between the maximum steady state plasma concentration (C_{ss:max}) and the average steady state plasma concentration (Cₛₛ) is 5% to 25%. One skilled in the art would appreciate that Cₛₛ is generally achieved within two to three weeks (usually three weeks) after the start of cholinesterase inhibitor therapy, and that Cₘₐₓ is generally achieved within hours of administration of the cholinesterase inhibitor. In another embodiment, the invention provides sustained-release formulations comprising at least one cholinesterase inhibitor, where the difference between C_{ss:max} and Cₛₛ is 5% to 23%; 5% to 22%; 5% to 21%; 5% to 20%; 5% to 18%; 5% to 15%; 5% to 12%; 5% to 11%; 5% to 10%; 5% to 9%; or 5% to 8%. In one embodiment, the cholinesterase inhibitor is donepezil, a pharmaceutically acceptable salt thereof, and/or a stereoisomer thereof. In another embodiment, the invention provides sustained-release formulations comprising at least one cholinesterase inhibitor, where the difference between the maximum steady state plasma concentration (C_{ss:max}) and the steady state plasma concentration (Cₛₛ) is 10% to 25%; 10% to 23%; or 10% to 20%. In one embodiment, the cholinesterase inhibitor is donepezil, a pharmaceutically acceptable salt thereof and/or a stereoisomer thereof. The sustained release formulations may comprise from 1 milligram to 50 milligrams of donepezil; or from 10 milligrams to 25 milligrams donepezil. In other embodiments, the invention provides sustained release formulations comprising 14 milligrams, 15 milligrams, 20 milligrams, or 23 milligrams donepezil.

In another embodiment, the invention provides sustained-release formulations comprising 10 milligrams to 25 milligrams of at least one cholinesterase inhibitor, where the difference between C_{ss:max} and Cₛₛ is 15% to 23%; 15% to 22%; 15% to 21%; or 18% to 21%. In other embodiments, the invention provides formulations comprising 14 milligrams, 15 milligrams, 20 milligrams, or 23 milligrams donepezil. In one embodiment, the cholinesterase inhibitor is donepezil, a pharmaceutically acceptable salt thereof, and/or a stereoisomer thereof.

In another embodiment, the invention provides sustained-release formulations comprising 10 milligrams to 25 milligrams of at least one cholinesterase inhibitor, where the difference between C_{ss:max} and Cₛₛ is 5% to 15%; 5% to 12%; or 5% to 11%. The sustained release formulations may comprise 10 milligrams to 25 milligrams of a cholinesterase inhibitor. In one embodiment, the cholinesterase inhibitor is donepezil, a pharmaceutically acceptable salt thereof, and/or a stereoisomer thereof. In other embodiments, the invention provides orally administrable sustained release formulations comprising 14 milligrams, 15 milligrams, 20 milligrams, or 23 milligrams donepezil.

In another embodiment, the invention provides sustained-release formulations comprising 10 milligrams to 25 milligrams of at least one cholinesterase inhibitor, where the difference between C_{ss:max} and Cₛₛ is 5% to 12%; 5% to 10%; 5% to 9%; or 5% to 8%. In one embodiment, the cholinesterase inhibitor is donepezil, a pharmaceutically acceptable salt thereof, and/or a stereoisomer thereof. In other embodiments, the invention provides orally administrable sustained release formulations comprising 14 milligrams, 15 milligrams, 20 milligrams, or 23 milligrams donepezil.

In another embodiment, the invention provides sustained-release formulations comprising 10 milligrams of at least one cholinesterase inhibitor where the steady state plasma concentration (Cₛₛ) is from 20 ng/ml to 30 ng/ml; from 20 ng/ml to 29 ng/ml; or from 20 ng/ml to 28 ng/ml. In one embodiment, the cholinesterase inhibitor is donepezil, a pharmaceutically acceptable salt thereof and/or a stereoisomer thereof.

In another embodiment, the invention provides sustained-release formulations comprising 14 milligrams of at least one cholinesterase inhibitor where the steady state plasma concentration (Cₛₛ) is from 28 ng/ml to 42 ng/ml; or from 30 ng/ml to 40 ng/ml. In one embodiment, the cholinesterase inhibitor is donepezil, a pharmaceutically acceptable salt thereof and/or a stereoisomer thereof.

In another embodiment, the invention provides sustained-release formulations comprising 15 milligrams of at least one cholinesterase inhibitor where the steady state plasma concentration (Cₛₛ) is from 30 ng/ml to 45 ng/ml; from 32 ng/ml to 45 ng/ml; from 32 ng/ml to 44 ng/ml; from 32 ng/ml to 43 ng/ml; or from 32 ng/ml to 42 ng/ml. In one embodiment, the cholinesterase inhibitor is donepezil, a pharmaceutically acceptable salt thereof, and/or a stereoisomer thereof.

In another embodiment, the invention provides sustained-release formulations comprising 20 milligrams of at least one cholinesterase inhibitor where the steady state plasma concentration (Cₛₛ) is from 45 ng/ml to 57 ng/ml; from 45 ng/ml to 56 ng/ml; from 45 ng/ml to 56 ng/ml; from 45 ng/ml to 55 ng/ml; from 48 ng/ml to 53 ng/ml; from 50 ng/ml to 52 ng/ml; or 51 ng/ml. In one embodiment, the cholinesterase inhibitor is donepezil, a pharmaceutically acceptable salt thereof and/or a stereoisomer thereof.

In another embodiment, the invention provides sustained-release formulations comprising 23 milligrams of at least one cholinesterase inhibitor where the steady state plasma concentration (Cₛₛ) is from 46 ng/ml to 69 ng/ml; or from 50 ng/ml to 60 ng/ml. In one embodiment, the cholinesterase inhibitor is donepezil, a pharmaceutically acceptable salt thereof and/or a stereoisomer thereof.

In another embodiment, the invention provides sustained-release formulations comprising at least one cholinesterase inhibitor, where the difference between the maximum steady state plasma concentration (C_{ss:max}) and the minimum steady state plasma concentration (C_{ss:min}) is less than 40%. In another embodiment, the invention provides sustained-release formulations comprising at least one cholinesterase inhibitor, where the difference between C_{ss:max} and C_{ss:min} is from 5% to 35%; from 5% to 30%; from 5% to 25%; from 5% to 20%; from 5% to 15%, or from 5% to 10%. The sustained release formulations may comprise from 10 to 25 milligrams of a cholinesterase inhibitor. In one embodiment, the cholinesterase inhibitor is donepezil, a pharmaceutically acceptable salt thereof and/or a stereoisomer thereof. The sustained-release formulations may comprise from 14 to 23 milligrams donepezil. In other embodiments, the formulations may comprise 14 milligrams, 15 milligrams, 20 milligrams, or 23 milligrams donepezil.

In another embodiment, the invention provides sustained-release formulations comprising at least one cholinesterase inhibitor, where the ratio of the maximum steady state plasma concentration (C_{ss:max}) to the minimum steady state plasma concentration (C_{ss:min}) is from 1.0 to 1.5; from 1.0 to 1.4; from 1.0 to 1.3; or from 1.0 to 1.2. In one embodiment, the lower value is 1.05. In yet another embodiment, the ratio of the maximum steady state plasma concentration (C_{ss:max}) to the minimum steady state plasma concentration (C_{ss:min}) is 1.05 to 1.4; 1.1 to 1.3; or 1.2. The sustained release formulations may comprise from 10 to 25 milligrams of a cholinesterase inhibitor. In one embodiment, the cholinesterase inhibitor is donepezil, a pharmaceutically acceptable salt thereof and/or a stereoisomer thereof. In other embodiments, the formulations may comprise 14 milligrams, 15 milligrams, 20 milligrams, or 23 milligrams donepezil.

In another embodiment, the invention provides sustained-release formulations comprising at least one cholinesterase inhibitor wherein Cₘₐₓ of the sustained-release formulation is at least 20% less than Cₘₐₓ of a conventional, immediate release formulation. For example, sustained-release formulations of the invention comprising 5 mg donepezil would have a Cₘₐₓ of 27.3 ng/ml where Cₘₐₓ of a conventional, immediate release formulation was 34.1 ng/ml, as shown in Table A in the Background of the Invention. As another example, sustained-release formulations of the invention comprising 10 mg donepezil would have a Cₘₐₓ of 48.4 ng/ml where Cₘₐₓ of a conventional, immediate release formulation was 60.5 ng/ml, as shown in Table A in the Background of the Invention. In another embodiment, the invention provides sustained-release formulation of at least one cholinesterase inhibitor wherein Cₘₐₓ of the sustained-release formulation is at least 30% less than, 40% less than, 50% less than, 60% less than, 70% less than, or 75% less than Cₘₐₓ of a conventional, immediate release formulation. In another embodiment, the invention provides sustained-release formulations of at least one cholinesterase inhibitor wherein Cₘₐₓ of the sustained-release formulation is at least 80% less than, 85% less than, 90% less than, or 95% less than Cₘₐₓ of a conventional, immediate release formulation. In one embodiment, the cholinesterase inhibitor is donepezil, a pharmaceutically acceptable salt thereof and/or a stereoisomer thereof.

In one embodiment, the sustained-release formulations of the invention provide 35% or more cortical enzyme inhibition in the brain. In other embodiments, the sustained-release formulations of the invention provide 40% or more; 45% or more; or 50% or more cortical enzyme inhibition in the brain. The cortical enzyme inhibited is cholinesterase, preferably acetylcholinesterase. The result of enzyme inhibition is a longer half life or “period of life” for acetylcholine. Cholinesterase are blocked from catalyzing the metabolism of acetylcholine, thus increasing the number of acetylcholine molecules available to trigger cholinergic receptors in the key areas of the brain. The sustained release formulations may comprise from 10 to 25 milligrams of a cholinesterase inhibitor; or from 14 to 23 milligrams of a cholinesterase inhibitor. In other embodiments, the formulations may comprise 14 milligrams, 15 milligrams, 20 milligrams, or 23 milligrams donepezil. In one embodiment, the cholinesterase inhibitor is donepezil, a pharmaceutically acceptable salt thereof and/or a stereoisomer thereof.

There are no particular limitations on the basic drug used in the invention. The term “basic drug” includes the basic drug, pharmaceutically acceptable salts of the basic drug, stereoisomers of the basic drug, and pharmaceutically acceptable salts of the stereoisomers of the basic drug. Exemplary basic drugs that may be used in the invention include anti-dementia drugs such as NMDA receptor antagonists such as memantine (e.g. memantine hydrochloride), anti-dementia drugs such as cholinesterase inhibitors such as donepezil (e.g. donepezil hydrochloride), galantamine (e.g., galantamine hydrobromide), rivastigmine (e.g., rivastigmine tartrate), tacrine, and the like; anti-anxiety drugs such as flurazepam (e.g., flurazepam hydrochloride), alprazolam, tandospirone (e.g., tandospirone citrate), rilmazafone (e.g., rilmazafone hydrochloride) and the like; antihistamines such as diphenylpyraline (e.g., diphenylpyraline hydrochloride), chlorpheniramine (e.g., chlorpheniramine maleate), cimetidine, isothipendyl (e.g., isothipendyl hydrochloride) and the like; circulatory drugs such as phenylephrine (e.g., phenylephrine hydrochloride), procainamide (e.g., procainamide hydrochloride), quinidine (e.g., quinidine sulfate), isosorbide dinitrate, nicorandil and the like; anti-hypertensive drugs such as amlodipine (e.g., amlodipine besylate), nifedipine, nicardipine (e.g., nicardipine hydrochloride), nilvadipine, atenolol (e.g. atenolol hydrochloride), and the like; anti-psychotic drugs such as perospirone (e.g., perospirone hydrochloride), and the like; anti-bacterial agents such as levofloxacin and the like; antibiotics such as cephalexin, cefcapene pivoxil (e.g., cefcapene pivoxil hydrochloride), ampicillin and the like as well as sulfamethoxazole, tetracycline, metronidazole, indapamide, diazepam, papaverine (e.g., papaverine hydrochloride), bromhexine (e.g., bromhexine hydrochloride), ticlopidine (e.g., ticlopidine hydrochloride), carbetapentane (e.g., carbetapentane citrate), phenylpropanolamine (e.g., phenylpropanolamine hydrochloride), ceterizine (e.g., ceterizine hydrochloride), and other drugs and macrolide antibiotics such as erythromycin, dirithromycin, josamycin, midecamycin, kitasamycin, roxithromycin, rokitamycin, oleandomycin, miokamycin, flurithromycin, rosaramycin, azithromycin, clarithromycin and the like. One, two or more of the basic drugs may be contained in the matrix type sustained-release formulations of the invention.

Of these basic drugs, the anti-dementia drugs are preferred, and donepezil or a pharmaceutically acceptable salt thereof and/or memantine or a pharmaceutically acceptable salt thereof are particularly preferred. The matrix type sustained-release formulations of the invention are also suitable for basic drugs which have a narrow drug safety range or which produce adverse effects dependent on maximum blood concentration of the drug. There are no particular limitations on the anti-dementia drug contained in the matrix type sustained-release formulations of the invention, but from the standpoint of controlling release it is effective to use basic drugs which are less soluble in an alkaline aqueous solution than in an acidic aqueous solutions, and to use basic drugs where the solubility of the basic drugs for a pH of an aqueous solution changes near neutral pH. Exemplary basic drugs include those with a pKa from 7.0 to 12.0; from 7.5 to 11.0; from 8.0 to 10.5; or from 8.5 to 10.5. Such pKa ranges would include drugs like donepezil hydrochloride (pKa=8.90) and memantine hydrochloride (pKa=10.27). In another embodiment of the invention, the basic drug has a pKa range from 8.5 to 9.5 (or from 8.5 to 9.0). In another embodiment of the invention, the basic drug has a pKa range from 10.0 to 10.5.

In one embodiment of the invention, the basic drug is a cholinesterase inhibitor. The cholinesterase inhibitor can be any in the art. The term “cholinesterase inhibitor” includes cholinesterase inhibitors, pharmaceutically acceptable salts of cholinesterase inhibitors, stereoisomers of cholinesterase inhibitors, and pharmaceutically acceptable salts of stereoisomers of cholinesterase inhibitors. Exemplary cholinesterase inhibitors include donepezil, tacrine, physostigmine, pyridostigmine, neostigmine, rivastigmine, galantamine, citicoline, velnacrine, huperzine (e.g., huperzine A), metrifonate, heptastigmine, edrophonium, phenserine, tolserine, phenethylnorcymserine, quilostigmine, ganstigmine, epastigmine, upreazine, TAK-147 (i.e., 3-[1-(phenylmethyl)-4-piperidinyl]-1-(2,3,4,5-tetrahydro-1H-1-benzazepin-8-yl)-1-propanone fumarate or other pharmaceutically acceptable salts thereof), T-82 (i.e., (2-[2-(1-benzylpiperidin-4-yl)ethyl]-2,3-dihydro-9-methoxy-1H-pyrrolo[3,4-b]quinolin-1-one hemifumarate or other pharmaceutically acceptable salts thereof)), and the like. In one embodiment, the cholinesterase inhibitor is donepezil, tacrine, galantamine, or rivastigmine.

In one embodiment, the cholinesterase inhibitors are compounds of formula (I), stereoisomers of the compounds of formula (I), pharmaceutically acceptable salts of the compounds of formula (I), or pharmaceutically acceptable salts of the stereoisomers of the compounds of formula (I): 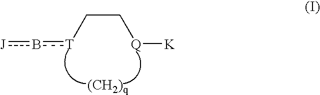
wherein J is (a) a substituted or unsubstituted group selected from (i) phenyl, (ii) pyridyl, (iii) pyrazyl, (iv) quinolyl, (v) cyclohexyl, (vi) quinoxalyl, and (vii) furyl; (b) a monovalent or divalent group, in which the phenyl can have one or more substituents selected from (i) indanyl, (ii) indanonyl, (iii) indenyl, (iv) indenonyl, (v) indanedionyl, (vi) tetralonyl, (vii) benzosuberonyl, (viii) indanolyl, and (ix) C₆H₅—CO—CH(CH₃)—; (c) a monovalent group derived from a cyclic amide compound; (d) a lower alkyl group; or (e) R²¹—CH═CH—, in which R²¹ is hydrogen or a lower alkoxycarbonyl group;

B is —(CHR²²)ᵣ—, —CO—(CHR²²)ᵣ—, —NR⁴—(CHR²²)ᵣ—, —CO—NR⁵—(CHR²²)ᵣ—, —CH═CH—(CHR²²)ᵣ—, —OCOO—(CHR²²)ᵣ—, —OOC—NH—(CHR²²)ᵣ—, —NH—CO—(CHR²²)ᵣ—CH₂—CO—NH—(CHR²²)ᵣ—, —(CH₂)₂—NH—(CHR²²)ᵣ—, —CH(OH)—(CHR²²)ᵣ—, ═(CH—CH═CH)_{b}—, ═CH—(CH₂)_{c}—, ═(CH—CH)_{d}═, —CO—CH═CH—CH₂—, —CO—CH₂—CH(OH)—CH₂—, —CH(CH₃)—CO—NH—CH₂—, —CH═CH═CO—NH—(CH₂)₂—, —NH—, —O—,—S—, a dialkylaminoalkyl-carbonyl or a lower alkoxycarbonyl;

R⁴ is hydrogen, lower alkyl, acyl, lower alkylsulfonyl, phenyl, substituted phenyl, benzyl, or substituted benzyl; R⁵ is hydrogen, lower alkyl or phenyl; r is zero or an integer of 1 to 10; R²² is hydrogen or methyl so that one alkylene group can have no methyl branch or one or more methyl branches; b is an integer of 1 to 3; c is zero or an integer of 1 to 9; d is zero or an integer of 1 to 5;

T is nitrogen or carbon;

Q is nitrogen, carbon or

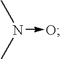

q is an integer of 1 to 3;

K is hydrogen, phenyl, substituted phenyl, arylalkyl in which the phenyl can have a substituent, cinnamyl, a lower alkyl, pyridylmethyl, cycloalkylalkyl, adamantanemethyl, furylmenthyl, cycloalkyl, lower alkoxycarbonyl or an acyl; and [symbol] is a single bond or a double bond.

In the compound of formula (I), J is preferably (a) or (b), more preferably (b). In the definition of (b), a monovalent group (2), (3) and (5) and a divalent group (2) are preferred. The group (b) preferably includes, for example, the groups having the formulae shown below: 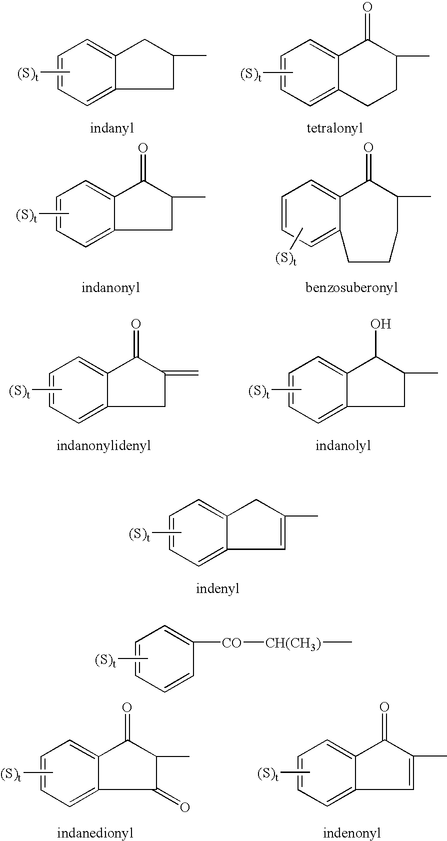
wherein t is an integer of 1 to 4; and each S is independently hydrogen or a substituent, such as a lower alkyl having 1 to 6 carbon atoms or a lower alkoxy having 1 to 6 carbon atoms. Among the substituents, methoxy is most preferred. The phenyl is most preferred to have 1 to 3 methoxy groups thereon. (S)ₜ, can form methylene dioxy groups or ethylene dioxy groups on two adjacent carbon atoms of the phenyl group. Of the above groups, indanonyl, indanedionyl and indenyl, optionally having substituents on the phenyl, are the most preferred.

In the definition of B, —(CHR²²)ᵣ—, —CO—(CHR²²)ᵣ—, ═(CH—CH═CH)_{b}—, ═CH—(CH₂)_{c}— and ═(CH—CH)_{d}═ are preferable. The group of ═(CHR²²)ᵣ in which R²² is hydrogen and r is an integer of 1 to 3, and the group of ═CH—(CH₂)_{c}— are most preferable. The preferable groups of B can be connected with (b) of J, in particular (b)(2). The ring containing T and Q in formula (I) can be 5-, 6- or 7-membered. It is preferred that Q is nitrogen, T is carbon or nitrogen, and q is 2; or that Q is nitrogen, T is carbon, and q is 1 or 3; or that Q is carbon, T is nitrogen and q is 2. It is preferable that K is a phenyl, arylalkyl, cinnamyl, phenylalkyl or a phenylalkyl having a substituent(s) on the phenyl.

In one embodiment, the cholinesterase inhibitors are compounds of formula (II), stereoisomers of the compounds of formula (II), pharmaceutically acceptable salts of the compounds of formula (II), or pharmaceutically acceptable salts of the stereoisomers of the compounds of formula (II): 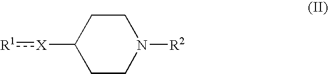
wherein R¹ is a (1) substituted or unsubstituted phenyl group; (2) a substituted or unsubstituted pyridyl group; (3) a substituted or unsubstituted pyrazyl group; (4) a substituted or unsubstituted quinolyl group; (5) a substituted or unsubstituted indanyl group; (6) a substituted or unsubstituted cyclohexyl group; (7) a substituted or unsubstituted quinoxalyl group; (8) a substituted or unsubstituted furyl group; (9) a monovalent or divalent group derived from an indanone having a substituted or unsubstituted phenyl ring; (10) a monovalent group derived from a cyclic amide compound; (11) a lower alkyl group; or (12) a group of the formula R³—CH═C—, where R³ is a hydrogen atom or a lower alkoxycarbonyl group;

X is —(CH₂)ₙ—, —C(O)—(CH₂)ₙ—, —N(R⁴)—(CH₂)ₙ—, —C(O)—N(R⁵)—(CH₂)ₙ—, —CH═CH—(CH₂)ₙ—, —O—C(O)—O—(CH₂)ₙ—, —O—C(O)—NH—(CH₂)ₙ—, —CH═CH—CH═CO—, —NH—C(O)—(CH₂)ₙ—, —CH₂—C(O)—NH—(CH₂)ₙ—, —(CH₂)₂—C(O)—NH—(CH₂)ₙ—, —CH(OH)—(CH₂)ₙ—, —C(O)—CH═CH—CH₂—, —C(O)—CH₂—CH(OH)—CH₂—, —CH(CH₃)—C(O)—NH—CH₂—, —CH═CH—C(O)—NH—(CH₂)₂—, a dialkylaminoalkylcarbonyl group, a lower alkoxycarbonyl group;

n is an integer of 0 to 6; R⁴ is a hydrogen atom, a lower alkyl group, an acyl group, a lower alkylsulfonyl group, a substituted or unsubstituted phenyl group, or a substituted or unsubstituted benzyl group; and R⁵ is a hydrogen atom a lower alkyl group or a phenyl group;

R² is a substituted or unsubstituted phenyl group; a substituted or unsubstituted arylalkyl group; a cinnamyl group; a lower alkyl group; a pyridylmethyl group; a cycloalkylalkyl group; an adamantanemethyl group; or a furoylmethyl group; and

[symbol] is a single bond or a double bond.

The term “lower alkyl group” means a straight or branched alkyl group having 1 to 6 carbon atoms. Exemplary “lower alkyl groups” include methyl, ethyl, propyl, isopropyl, butyl, isobutyl, sec-butyl, tert-butyl, pentyl (amyl), isopentyl, neopentyl, tert-pentyl, 1-methylbutyl, 2-methylbutyl, 1,2-dimethylpropyl, hexyl, isohexyl, 1-methylpentyl, 2-methyl-pentyl, 3-methylpentyl, 1,1-dimethylbutyl, 1,2-dimethylbutyl, 2,2-dimethylbutyl, 1,3-dimthyl-butyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, and the like. The lower alkyl group is preferably methyl, ethyl, propyl or isopropyl; more preferably methyl.

Specific examples of the substituents for the substituted or unsubstituted phenyl, pyridyl, pyrazyl, quinolyl, indanyl, cyclohexyl, quinoxalyl and furyl groups in the definition of R¹ include lower alkyl groups having 1 to 6 carbon atoms, such as methyl, ethyl, n-propyl, isopropyl, n-butyl, isobutyl, and tert-butyl groups; lower alkoxy groups corresponding to the above-described lower alkyl groups, such as methoxy and ethoxy groups; a nitro group; halogen atoms, such as chlorine, fluorine and bromine; a carboxyl group; lower alkoxycarbonyl groups corresponding to the above-described lower alkoxy groups, such as methoxycarbonyl, ethoxycarbonyl, isopropoxycarbonyl, n-propoxycarbonyl, and n-butyloxycarbonyl groups; an amino group; a lower monoalkylamino group; a lower dialkylamino group; a carbamoyl group; acylamino groups derived from aliphatic saturated monocarboxylic acids having 1 to 6 carbon atoms, such as acetylamino, propionylamino, butyrylamino, isobutyrylamino, valerylamino, and pivaloylamino groups; cycloalkyloxycarbonyl groups, such as a cyclohexyloxycarbonyl group; lower alkylaminocarbonyl groups, such as methylaminocarbonyl and ethylaminocarbonyl groups; lower alkylcarbonyloxy groups corresponding to the above-defined lower alkyl groups, such as methylcarbonyloxy, ethylcarbonyloxy, and n-propylcarbonyloxy groups; halogenated lower alkyl groups, such as a trifluoromethyl group; a hydroxyl group; a formyl group; and lower alkoxy lower alkyl groups, such as ethoxymethyl, methoxymethyl and methoxyethyl groups. The “lower alkyl groups” and “lower alkoxyl groups” in the above description of the substituent include all the groups derived from the above-mentioned groups. The substituent can be one to three of them, which can be the same or different.

When the substituent is a phenyl group, the following group is within the scope of the substituted phenyl group:

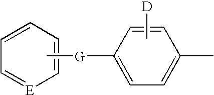

wherein G is —C(O)—, —O—C(O)—, —O—, —CH₂—NH—C(O)—, —CH₂—(O)—, —CH₂—SO₂—, —CH(OH)—, or —CH₂—S(→O)—; E is a carbon or nitrogen atom; and D is a substituent.

Preferred examples of the substituents (i.e., “D”) for the phenyl group include lower alkyl, lower alkoxy, nitro, halogenated lower alkyl, lower alkoxycarbonyl, formyl, hydroxyl, and lower alkoxy lower alkyl groups, halogen atoms, and benzyol and benzylsulfonyl groups. The substituent can be two or more of them, which can be the same or different. Preferred examples of the substituent for the pyridyl group include lower alkyl and amino groups and halogen atoms. Preferred examples of the substituent for the pyrazyl group include lower alkoxycarbonyl, carboxyl, acylamino, carbamoyl, and cycloalkyloxycarbonyl groups.

With respect to R¹, the pyridyl group is preferably a 2-pyridyl, 3-pyridyl, or 4-pyridyl group; the pyrazyl group is preferably a 2-pyrazinyl group; the quinolyl group is preferably a 2-quinolyl or 3-quinolyl group; the quinoxalinyl group is preferably a 2-quinoxalinyl or 3-quinoxalinyl group; and the furyl group is preferably a 2-furyl group.

Examples of monovalent or divalent groups derived from an indanone having an unsubstituted or substituted phenyl ring include those represented by formulas (A) and (B): 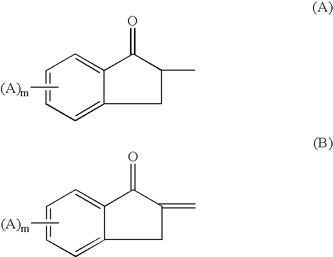
where m is an integer of from 1 to 4, and each A is independently a hydrogen atom, a lower alkyl group, a lower alkoxy group, a nitro group, a halogen atom, a carboxyl group, a lower alkoxycarbonyl group, an amino group, a lower monoalkylamino group, a lower dialkylamino group, a carbamoyl group, an acylamino group derived from aliphatic saturated monocarboxylic acids having 1 to 6 carbon atoms, a cycloalkyloxycarbonyl group, a lower alkylaminocarbonyl group, a lower alkylcarbonyloxy group, a halogenated lower alkyl group, a hydroxyl group, a formyl group, or a lower alkoxy lower alkyl group; preferably a hydrogen atom, a lower alkyl group or a lower alkoxy group; most preferably the indanone group is unsubstituted or substituted with 1 to 3 methoxy groups.

Examples of the monovalent group derived from a cyclic amide compound include quinazolone, tetrahydroisoquinolinone, tetrahydrobenzodiazepinone, and hexahydrobenzazocinone. However, the monovalent group can be any one having a cyclic amide group in the structural formula thereof, and is not limited to the above-described specific examples. The cyclic amide group can be one derived from a monocyclic or condensed heterocyclic ring. The condensed heterocyclic ring is preferably one formed by condensation with a phenyl ring. In this case, the phenyl ring can be substituted with a lower alkyl group having 1 to 6 carbon atoms, preferably a methyl group, or a lower alkoxy group having 1 to 6 carbon atoms, preferably a methoxy group.

Examples of the monovalent group include the following:

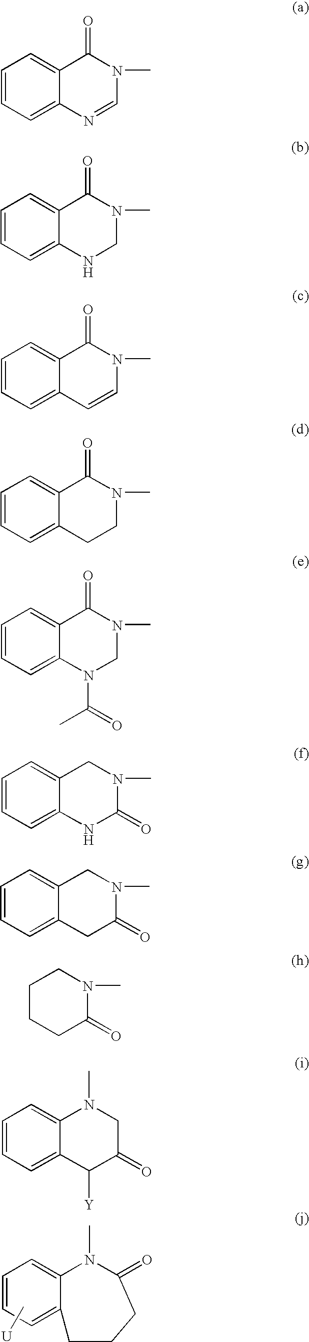

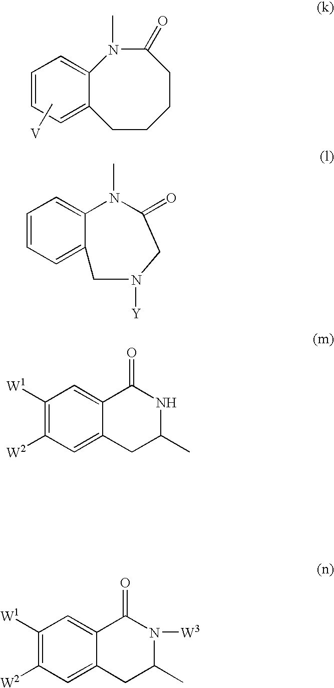

In the above formulae, Y is a hydrogen atom or a lower alkyl group; V and U are each a hydrogen atom or a lower alkoxy group (preferably dimethoxy); W¹ and W² are each a hydrogen atom, a lower alkyl group, or a lower alkoxy group; and W³ is a hydrogen atom or a lower alkyl group. The right hand ring in formulae (j) and (l) is a 7-membered ring, while the right hand ring in formula (k) is an 8-membered ring.

The most preferred examples of the above-defined R¹ include a monovalent group derived from an indanone having an unsubstituted or substituted phenyl group and a monovalent group derived from a cyclic amide compound.

The most preferred examples of the above-defined X include —(CH₂)ₙ—, an amide group, or groups represented by the above formulae where n is 2. Thus, it is most preferred that any portion of a group represented by the formula 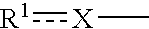
have a carbonyl or amide group.

The substituents involved in the expressions “a substituted or unsubstituted phenyl group” and “a substituted or unsubstituted arylalkyl group” in the above definition of R² are the same substituents as those described for the above definitions of a phenyl group, a pyridyl group, a pyrazyl group, a quinolyl group, an indanyl group, a cyclohexyl group, a quinoxalyl group or a furyl group in the definition of R¹.

The term “arylalkyl group” is intended to mean an unsubstituted benzyl or phenethyl group or the like.

Specific examples of the pyridylmethyl group include 2-pyridylmethyl, 3-pyridylmethyl, and 4-pyridylmethyl groups.

Preferred examples of R² include benzyl and phenethyl groups. The symbol [symbol] means a double or single bond. The bond is a double bond only when R¹ is the divalent group (B) derived from an indanone having an unsubstituted or substituted phenyl ring, while it is a single bond in other cases.

In one embodiment, the cholinesterase inhibitors are compounds of formula (III), stereoisomers of the compounds of formula (III), pharmaceutically acceptable salts of the compounds of formula (III), or pharmaceutically acceptable salts of the stereoisomers of the compounds of formula (III): 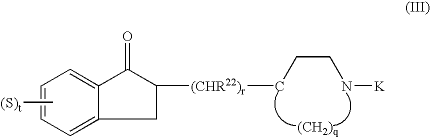
wherein r is an integer of 1 to 10; each R²² is independently hydrogen or methyl; K is a phenalkyl or a phenalkyl having a substituent on the phenyl ring; each S is independently a hydrogen, a lower alkyl group having 1 to 6 carbon atoms or a lower alkoxy group having 1 to 6 carbon atoms; t is an integer of 1 to 4; q is an integer of 1 to 3; with the proviso that (S)ₜ can be a methylenedioxy group or an ethylenedioxy group joined to two adjacent carbon atoms of the phenyl ring.

In other embodiments, the compound of formula (III) is 1-benzyl-4-((5,6-dimethoxy-1-indanon)-2-yl)methylpiperidine; 1-benzyl-4-((5,6-dimethoxy-1-indanon)-2-ylidenyl)methyl-piperidine; 1-benzyl-4-((5-methoxy-1-indanon)-2-yl)methylpiperidine; 1-benzyl-4-((5,6-diethoxy-1-indanon)-2-yl)methylpiperidine; 1-benzyl-4-((5,6-methnylenedioxy-1-indanon)-2-yl)methylpiperidine; 1-(m-nitrobenzyl)-4-((5,6-dimethoxy-1-indanon)-2-yl)methylpiperidine; 1-cyclohexylmethyl-4-((5,6-dimethoxy-1-indanon)-2-yl)methylpiperidine; 1-(m-fluorobenzyl)-4-((5,6-dimethoxy-1-indanon)-2-yl)methylpiperidine; 1 -benzyl-4-((5 ,6-dimethoxy-1 -indanon)-2 -yl)propylpiperidine; 1-benzyl-4-((5-isopropoxy-6-methoxy-1-indanon)-2-yl)methylpiperidine; 1-benzyl-4-((5,6-dimethoxy-1-oxoindanon)-2-yl)propenylpiperidine; pharmaceutically acceptable salts of one or more of the foregoing; stereoisomers of one or more of the foregoing; or pharmaceutically acceptable salts of stereoisomers of one or more of the foregoing.

In other embodiments, the compound of formula (III) is 1-benzyl-4-((5,6-dimethoxy-1-indanon)-2-yl)methylpiperidine; a pharmaceutically acceptable salt thereof; a stereoisomer thereof; or a pharmaceutically acceptable salt of a stereoisomer thereof; which is represented by formula (IV):

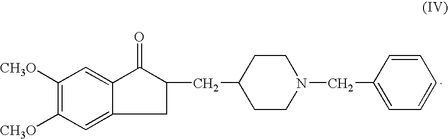

In still other embodiments, the compound of formula (III) is 1-benzyl-4-((5,6-dimethoxy-1-indanon)-2-yl)methylpiperidine hydrochloride or a stereoisomer thereof, which is also known as donepezil hydrochloride, and which is represented by formula (IVa):

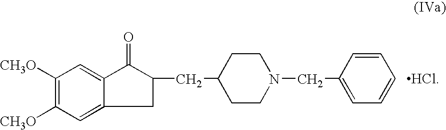

The compounds of the invention can have an asymmetric carbon atom(s), depending upon the substituents, and can have stereoisomers, which are within the scope of the invention. For example, donepezil or pharmaceutically acceptable salts thereof can be in the forms described in Japanese Patent Application Nos. 4-187674 and 4-21670, the disclosures of which are incorporated by reference herein in their entirety.

Japanese Patent Application No. 4-187674 describes a compound of formula (V): 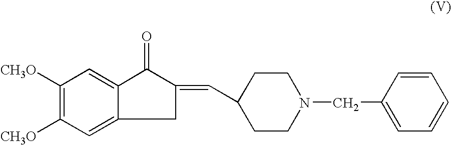
which can be in the form of a pharmaceutically acceptable salt, such as a hydrochloride salt.

Japanese Patent Application No. 4-21670 describes compounds of formula (VI): 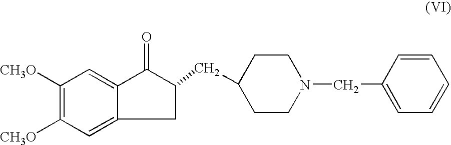
which can be in the form of a pharmaceutically acceptable salt, such as a hydrochloride salt; and compounds of formula (VII): 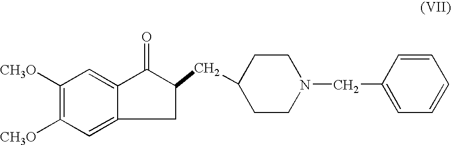
which can be in the form of a pharmaceutically acceptable salt, such as a hydrochloride salt; and compounds of formula (VIII):

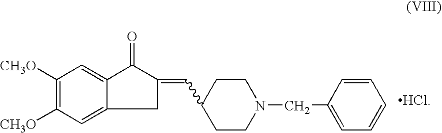

The basic drugs of the invention (e.g., cholinesterase inhibitors) may be administered in the form of pharmaceutically acceptable salts. Pharmaceutically acceptable salts are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the compounds of the invention can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the compounds of the invention can be made in the form of any other pharmaceutically acceptable salt (e.g., carbonates, mesylates, tartrates, citrates, tosylates, and the like).

The basic drugs of the invention, including cholinesterase inhibitors, are commercially available or can be prepared by processes known in the art, such as those described, for example, in U.S. Pat. No. 4,895,841, WO 98/39000, and Japanese Patent Application Nos. 4-187674 and 4-21670, the disclosures of which are incorporated by reference herein in their entirety. Memantine hydrochloride is commercially available as EBIXA® from H. Lunbeck A/S, Copenhagen, Denmark.

In addition to at least one cholinesterase inhibitor, the sustained release formulations of the invention may comprise other active ingredients that are useful for the disease being treated. For example, the sustained release formulations may further comprise memantine or pharmaceutically acceptable salts thereof for treating Alzheimer's disease. In other embodiments, the sustained release formulations may comprise one or more NSAIDs, such as naproxen, celecoxib, or rofecoxib for treating Alzheimer's disease. In still other embodiments, the sustained release formulations may further comprise vitamin E and/or ginkgo biloba for treating Alzheimer's disease. In still other embodiments, the sustained release formulations may comprise two or more cholinesterase inhibitors.

The dosage regimen for treating and preventing the diseases described herein with the basic compounds (e.g., cholinesterase inhibitors) can be selected in accordance with a variety of factors, including the age, weight, sex, and medical condition of the patient, the route of administration, pharmacological considerations such as the activity, efficacy, pharmacokinetic and toxicology profiles of the drugs, and whether a drug delivery system is used.

The cholinesterase inhibitors (e.g., donepezil) can be administered in doses of 0.01 to 50 milligrams per day, 0.1 to 40 milligrams per day; from 1 to 30 milligrams per day; from 5 to 25 milligrams per day; or from 10 to 23 milligrams per day. In another embodiment, the cholinesterase inhibitor is administered in an amount of 5 milligrams per day, 6 milligrams per day, 7 milligrams per day, 7.5 milligrams per day, 8 milligrams per day, 9 milligrams per day, 10, milligrams per day, 11, milligrams per day, 12, milligrams per day, 12.5 milligrams per day, 13 milligrams per day, 14 milligrams per day, 15 milligrams per day, 16 milligrams per day, 17 milligrams per day, 17.5 milligrams per day, 18 milligrams per day, 19 milligrams per day, 20 milligrams per day, 21 milligrams per day, 22 milligrams per day, 22.5 milligrams per day, 23 milligrams per day, 24 milligrams per day, 25 milligrams per day, 26 milligrams per day, 27 milligrams per day, 27.5 milligrams per day, or 28 milligrams per day. In other embodiments, the cholinesterase inhibitors are administered in amounts of 5 mg per day, 7.5 mg per day, 10 mg per day, 12.5 mg per day, 14 mg per day, 15 mg per day, 17.5 mg per day, 20 mg per day, 22.5 mg per day, 23 mg per day, 25 mg per day or 27.5 mg per day. In other embodiments, the cholinesterase inhibitors (e.g., donepezil) are administered in amounts of 10 mg per day, 12.5 mg per day, 14 mg per day, 15 mg per day, 17.5 mg per day, 20 mg per day, 22.5 mg per day, 23 mg per day, or 25 mg per day. In still other embodiments, the cholinesterase inhibitors (e.g., donepezil) are administered in amounts of 10 mg per day, 14 mg per day, 15 mg per day, 20 mg per day, or 23 mg per day. In still other embodiments, the cholinesterase inhibitors (e.g., donepezil) are administered in amounts of 14 mg per day, 15 mg per day, 20 mg per day, or 23 mg per day. The doses can be administered in one to four portions over the course of a day, preferably once a day.

The dose of NMDA receptor antagonist (e.g., memantine or pharmaceutically acceptable salts thereof (e.g., hydrochloride)) can be administered in amounts from 0.5 milligram to 100 milligrams per day. In other embodiments, memantine is administered in amounts from 0.1 milligram to 40 milligrams per day; in amounts from 1 milligram to 30 milligrams per day; or in amounts from 2 milligrams to 25 milligrams per day. In other embodiments, memantine is administered in amounts of 5 milligrams, 10 milligrams, 15 milligrams or 20 milligrams per day. The doses can be administered in one to four portions per day, preferably once a day.

The dose of rivastigmine or a pharmaceutically acceptable salt thereof (e.g., tartrate) is from 0.01 to 50 mg/day; from 0.1 to 30 mg/day; from 1 to 20 mg/day; or from 1 to 15 mg/day. The does of galantamine or pharmaceutically acceptable salt thereof (e.g., hydrobromide) is from 0.01 to 50 mg/day; from 0.1 to 40 mg/day, from 1 to 30 mg/day; or from 2 to 25 mg/day. The doses can be administered in one to four portions over the course of a day, preferably once a day. The doses can be administered in one to four portions over the course of a day, preferably once a day.

In one embodiment, the invention provides matrix type sustained release formulations. The matrix type sustained release formulations are capable not only of inhibiting initial drug bursts (i.e., immediate rapid release of the drug after dissolution) but also of ensuring dissolution with low pH dependence at early stages of dissolution in dissolution tests. At the same time, the invention provides matrix type sustained release formulations wherein, as the dissolution test proceeds, the ratio of the dissolution rate of the drug in an acidic solution to the dissolution rate in a neutral solution (i.e., dissolution rate in the acidic solution: dissolution rate in the neutral solution) decreases with dissolution time at the late stage of dissolution, as compared to the early stage of dissolution.

Taking the pH environment of the body into consideration, there are demands for sustained release formulations containing basic drugs (e.g., cholinesterase inhibitors) which inhibit unexpected increases in blood concentrations associated with rapid dissolution of the drug from formulations and which offer decreased risks of reduced bioavailability associated with the sustained release characteristics. There are demands in the art for matrix type sustained release formulations containing drugs which not only inhibit the initial drug burst (i.e., immediate rapid drug release after dissolution) in dissolution tests, but also ensure dissolution with low pH dependence of the drug at the early stage of dissolution, and wherein as the dissolution test proceeds, the dissolution speed with low pH dependence in a neutral pH solution is high at the late stage of dissolution. Accordingly, this is a demand for matrix type sustained release formulations containing a drug in which the ratio of the dissolution rate of the drug in an acidic solution to the dissolution rate of the drug in a neutral solution (dissolution rate in the acidic solution:dissolution rate in the neutral solution) decreases with dissolution time at the early stage of dissolution, as compared to the late stage of dissolution. In particular, there are demands for matrix type sustained release formulations which are capable of controlling the dissolution of the drug, so that the solubility of the drug decreases greatly with increased pH from a near-neutral to a weakly alkaline.

There are no particular limitations on the solubility of the basic drug used in the invention with respect to acidic aqueous solutions, neutral aqueous solutions or basic solutions, but the solubility of the basic drug in the acidic aqueous solution and the neutral aqueous solution is higher than its solubility in the basic aqueous solution. Herein, for use in preparations of these aqueous solutions, examples for this use include, but are not limited to, a phosphate buffer (e.g., buffers prepared with 50 mM sodium phosphate solution and hydrochloric acid), buffers such as G. L. Miller's buffer, Atkins-Pantin's buffer, Good's buffer, and the like, 0.1 N hydrochloric acid, 0.1 mol/l sodium hydroxide solution, and the like. The solubility refers to the solubility when the solution temperature is 25° C.

The term “solubility in an acidic aqueous solution” means the solubility of the basic drug in a solution exhibiting an acidic property when dissolving the basic drug in a buffer or the like. The term “solubility in a neutral aqueous solution” means the solubility of the basic drug in a solution exhibiting a neutral property when dissolving the basic drug in a buffer or the like. The term “solubility in a basic aqueous solution” means the solubility of the basic drug in a solution exhibiting a basic property when dissolving the basic drug in a buffer or the like.

By way of example, the basic drug of the invention has a higher solubility in an acidic aqueous solution (pH 3.0) and a neutral aqueous solution (pH 6.0) than in a basic aqueous solution (pH 8.0). The term “solubility in an acidic aqueous solution (pH 3.0)” means the solubility of the basic drug in a solution having a pH of 3.0 when dissolving the basic drug in a buffer or the like. The term “solubility in a neutral aqueous solution (pH 6.0)” means the solubility of the basic drug in a solution having a pH of 6.0 when dissolving the basic drug in a buffer or the like. The term “solubility in a basic aqueous solution (pH 8.0)” means the solubility of the basic drug in a solution having a pH of 8.0 when dissolving the basic drug in a buffer or the like.

By way of another example, the basic drug used in the invention has a higher solubility in a 0.1 N hydrochloric acid solution and a neutral aqueous solution (pH 6.0) than in a basic aqueous solution (pH 8.0). The term “solubility in a 0.1 N hydrochloric acid solution” means the solubility of the basic drug when dissolving the basic drug in a 0.1 N hydrochloric acid solution. For example, donepezil hydrochloride dissolved in a 0.1 N hydrochloric acid solution shows a pH range of 1 to 2.

The basic drug of the invention has a solubility in a 0.1 N hydrochloric acid solution and a neutral aqueous solution (pH 6.0) that is higher than in a basic aqueous solution (pH 8.0) and a solubility in the neutral aqueous solution (pH 6.8) is at least twice its solubility in a basic aqueous solution (pH 8.0), and is not more than half its solubility in a neutral aqueous solution (pH 6.0). The term “solubility in a neutral aqueous solution (pH 6.8)” means a solubility of the basic drug in a solution having a pH of 6.8 when dissolving the basic drug in a buffer or the like.

There are no particular limitations on the basic drug as long as the solubility in a 0.1 N hydrochloric acid solution and a neutral aqueous solution (pH 6.0) is 1 mg/ml or more; and the solubility of the basic drug in a basic aqueous solution (pH 8.0) is 0.2 mg/ml or less, and the solubility of the basic drug in a neutral aqueous solution (pH 6.8) is two or more times its solubility in a basic aqueous solution (pH 8.0) and is not more than half its solubility in a neutral aqueous solution (pH 6.0). The solubility of the basic drug in a 0.1 N hydrochloric acid solution and the neutral aqueous solution (pH 6.0) is not particularly limited as long as the solubility is 1 mg/ml or more. The solubility in a 0.1 N hydrochloric acid solution and a neutral aqueous solution (pH 6.0) may be from 1 to 1000 mg/ml; from 5 to 200 mg/ml; from 5 to 100 mg/ml; or from 10 to 80 mg/ml. The solubility of the basic drug in the basic aqueous solution (pH 8.0) is not particularly limited as long as it is 0.2 mg/ml or less. The solubility in the basic aqueous solution (pH 8.0) may be from 0.0001 to 0.2 mg/ml; from 0.0005 to 0.1 mg/ml; from 0.001 to 0.05 mg/ml; or from 0.002 to 0.03 mg/ml. The solubility of the basic drug in the neutral aqueous solution (pH 6.8) is not particularly limited as long as the solubility is at least twice its solubility in a basic aqueous solution (pH 8.0) and is not more than half its solubility in a neutral aqueous solution (pH 6.0). In one embodiment, the solubility of the basic drug in the neutral aqueous solution (pH 6.8) is at least three times its solubility in a basic aqueous solution (pH 8.0) and is not more than one-third its solubility in a neutral aqueous solution (pH 6.0). In one embodiment, the solubility of the basic drug in the neutral aqueous solution (pH 6.8) is at least five times its solubility in a basic aqueous solution (pH 8.0) and is not more than one-fifth its solubility in a neutral aqueous solution (pH 6.0). In one embodiment, the solubility of the basic drug in the neutral aqueous solution (pH 6.8) is at least ten times its solubility in a basic aqueous solution (pH 8.0) and is not more than one-tenth its solubility in a neutral aqueous solution (pH 6.0).

The solubility of the basic drug of the invention in a 0.1 N hydrochloric acid solution and a 50 mM phosphate buffer (pH 6.0) is higher than its solubility in a 50 mM phosphate buffer (pH 8.0). The term “solubility in a 50 mM phosphate buffer (pH 6.0)” means a solubility of the basic drug in a 50 mM phosphate buffer having a pH of 6.0 when dissolving the basic drug in a 50 mM phosphate buffer. The term “solubility in a 50 mM phosphate buffer (pH 8.0)” means a solubility of the basic drug in a 50 mM phosphate buffer having a pH of 8.0 when dissolving the basic drug in a 50 mM phosphate buffer.

The solubility of the basic drug in a 0.1 N hydrochloric acid solution and a 50 mM phosphate buffer (pH 6.0) is higher than its solubility in a 50 mM phosphate buffer (pH 8.0), and the solubility in the 50 mM phosphate buffer (pH 6.8) is at least twice its solubility in a 50 mM phosphate buffer (pH 8.0) and is not more than half its solubility in a 50 mM phosphate buffer (pH 6.0). The solubility of the basic drug in a 0.1 N hydrochloric acid solution and a 50 mM phosphate buffer (pH 6.0) is 1 mg/ml or more; and the solubility of the basic drug in a 50 mM phosphate buffer (pH 8.0) is 0.2 mg/ml or less; and the solubility of the basic drug in a 50 mM phosphate buffer (pH 6.8) is at least twice its solubility in a 50 mM phosphate buffer (pH 8.0) and is not more than half its solubility in a 50 mM phosphate buffer (pH 6.0). The solubility of the basic drug in a 0.1 N hydrochloric acid solution and a 50 mM phosphate buffer (pH 6.0) is at least 1 mg/ml or more; from 1 mg/ml to 1000 mg/ml; from 5 to 200 mg/ml; from 5 to 100 mg/ml; or from 10 to 80 mg/ml. The solubility of the basic drug in a 50 mM phosphate buffer (pH 8.0) is 0.2 mg/ml or less; from 0.0001 to 0.2 mg/ml; from 0.0005 to 0.1 mg/ml; from 0.001 to 0.05 mg/ml; or from 0.002 to 0.03 mg/ml. The solubility of the basic drug in a 50 mM phosphate buffer (pH 6.8) is not particularly limited as long as the solubility is at least twice its solubility in a 50 mM phosphate buffer (pH 8.0) and is not more than half its solubility in a 50 mM phosphate buffer (pH 6.0). The solubility of the basic drug in a 50 mM phosphate buffer (pH 6.8) is at least three times; at least five times; or at least ten times, its solubility in a 50 mM phosphate buffer (pH 8.0) and is not more than one-third; not more than one-fifth; or not more than one-tenth, respectively, its solubility in a 50 mM phosphate buffer (pH 6.0).

Donepezil hydrochloride has a solubility of 11 to 16 mg/ml in an acidic aqueous solution (pH 3.0) and a neutral aqueous solution (pH 6.0) and 0.1 mg/ml or less in a basic aqueous solution (pH 8.0). Donepezil hydrochloride is a weakly basic drug having one tertiary amino group, and is characterized by its solubility in a neutral aqueous solution (pH 6.8) being at least twice its solubility in a basic aqueous solution (pH 8.0) and not more than half its solubility in a neutral aqueous solution (pH 6.0). Alternatively, donepezil hydrochloride has a solubility of 11 to 16 mg/ml in a 0.1 N hydrochloric acid solution and a 50 mM phosphate buffer (pH 6.0) and 0.1 mg/ml or less in a 50 mM phosphate buffer (pH 8.0), and is characterized by its solubility in a 50 mM phosphate buffer (pH 6.8) being at least twice its solubility in a 50 mM phosphate buffer (pH 8.0) and not more than half its solubility in a 50 mM phosphate buffer (pH 6.0).

As a result of exhaustive research, the inventors have discovered that the desired objects can be achieved, for example, with the formulations shown below.

(I) The invention provides matrix type sustained release formulations comprising: (1) a basic drug which has higher solubility in a 0.1 N hydrochloric acid solution and a neutral aqueous solution (pH 6.0) than in a basic aqueous solution (pH 8.0); and (2) at least one enteric polymer. In one embodiment, the neutral aqueous solution is a 50 mM phosphate buffer, and the basic aqueous solution is 50 mM phosphate buffer.

(II) The invention provides matrix type sustained release formulations as described in (I) above, wherein in a dissolution test according to the Japanese Pharmacopoeia (14th Edition) paddle method, the ratio of the dissolution rate of the basic drug in the 0.1 N hydrochloric acid solution to the dissolution rate of the basic drug in a 50 mM phosphate buffer (pH 6.8) decreases with dissolution time until a dissolution time at which the dissolution rate of the drug in the 50 mM phosphate buffer (pH 6.8) is 90%.

(III) In another embodiment, the invention provides matrix type sustained release formulations as described in (I) or (II) above, wherein in the dissolution test according to the Japanese Pharmacopoeia (14th Edition) paddle method, the dissolution rate of the basic drug in a 0.1 N hydrochloric acid solution is not more than 60% at a dissolution time of 1 hour. Alternatively, the dissolution rate of the basic drug in the 0.1 N hydrochloric acid solution is not more than 50% at a dissolution time of 1 hour; or not more than 40% at a dissolution time of 1 hour.

(IV) In another embodiment, the invention provides matrix type sustained release formulations as described in one or more of (I), (II) and (III) above, wherein in the dissolution test according to the Japanese Pharmacopoeia (14th Edition) paddle method, the ratio of the dissolution rate of the basic drug in the 0.1 N hydrochloric acid solution to the dissolution rate of the drug in the 50 mM phosphate buffer (pH 6.8) is from 0.3 to 1.5 at a dissolution time of 3 hours. Alternatively, the ratio of the dissolution rate is from 0.3 to 1.4; from 0.3 to 1.3; or from 0.3 to 1.2.

(V) In another embodiment, the invention provides matrix type sustained release formulations as described in one or more of (I), (II), (III) and (IV) above, wherein in the dissolution test according to the Japanese Pharmacopoeia (14th Edition) paddle method, the ratio of the dissolution rate of the basic drug in the 0.1 N hydrochloric acid solution is not more than 60% at a dissolution time of 1 hour, and the ratio of the dissolution rate of the basic drug in the 0.1 N hydrochloric acid solution to the dissolution rate of the basic drug in the 50 mM phosphate buffer (pH 6.8) is from 0.3 to 1.5 at a dissolution time of 3 hours. Alternatively, the dissolution rate of the basic drug in the 0.1 N hydrochloric acid solution is not more than 50% at a dissolution time of 1 hour and the ratio of the dissolution rate of the basic drug in the 0.1 N hydrochloric acid solution to the dissolution rate of the drug in the 50 mM phosphate buffer (pH 6.8) is from 0.3 to 1.4. Alternatively, the dissolution rate of the basic drug in the 0.1 N hydrochloric acid solution is not more than 40% at a dissolution time of 1 hour and the ratio of the dissolution rate of the basic drug in the 0.1 N hydrochloric acid solution to the dissolution rate of the basic drug in the 50 mM phosphate buffer (pH 6.8) is from 0.3 to 1.2.

The matrix type sustained release formulations of the invention may also comprise at least one water-insoluble polymer. For example, the invention provides matrix type sustained release formulations comprising: (1) at least one basic drug which has a higher solubility in a 0.1 N hydrochloric acid solution and a 50 mM phosphate buffer (pH 6.0) than in a 50 mM phosphate buffer (pH 8.0); (2) at least one enteric polymer; and (3) at least one water-insoluble polymer.

In another embodiment, the invention provides matrix type sustained release formulations comprising (1) at least one basic drug wherein the solubility of the basic drug in the neutral aqueous solution (pH 6.8) is at least twice its solubility in the basic aqueous solution (pH 8.0) and is not more than half its solubility in the neutral aqueous solution (pH 6.0); (2) at least one enteric polymer; and (3) optionally at least one water insoluble polymer. In another embodiment, the invention provides matrix type sustained release formulations comprising (1) at least one basic drug wherein the solubility of the basic drug in a 50 mM phosphate buffer (pH 6.8) is at least twice its solubility in a 50 mM phosphate buffer (pH 8.0) and is not more than half its solubility in a 50 mM phosphate buffer (pH 6.0); (2) at least one enteric polymer; and (3) optionally at least one water insoluble polymer.

In another embodiment, the invention provides matrix type sustained release formulations comprising (1) at least one basic drug wherein the solubility of the basic drug in a 0.1 N hydrochloric acid solution and a 50 mM phosphate buffer (pH 6.0) is 1 mg/ml or more and the solubility of the basic drug in a 50 mM phosphate buffer (pH 8.0) is 0.2 mg/ml or less; (2) at least one enteric polymer; and (3) optionally at least one water insoluble polymer.

According to one embodiment, the invention provides matrix type sustained release formulations comprising: (1) at least one basic drug wherein the solubility is 1 mg/ml or more in a 0.1 N hydrochloric acid solution and a 50 mM phosphate buffer (pH 6.0), and is 0.2 mg/ml or less in a 50 mM phosphate buffer (pH 8.0), and the solubility of the basic drug in a 50 mM phosphate buffer (pH 6.8) is at least twice its solubility in a 50 mM phosphate buffer (pH 8.0), and is not more than half its solubility in a 50 mM phosphate buffer (pH 6.0); (2) at least one enteric polymer; and (3) optionally at least one water-insoluble polymer.

According to another embodiment, the invention provides matrix type sustained release formulations comprising (1) at least one basic drug wherein the solubility of the basic drug is 1 mg/ml or more in a 0.1 N hydrochloric acid solution and a 50 mM phosphate buffer (pH 6.0) and is 0.2 mg/ml or less in a 50 mM phosphate buffer (pH 8.0), and the solubility of the basic drug in a 50 mM phosphate buffer (pH 6.8) is at least twice its solubility in a 50 mM phosphate buffer (pH 8.0) and is not more than half its solubility if a 50 mM phosphate buffer (pH 6.0); (2) at least one enteric polymer; and (3) optionally at least one water insoluble polymer.

In another embodiment, the invention provides matrix type sustained release formulations comprising (1) at least one basic drug which has a higher solubility in a 0.1 N hydrochloric acid solution and a neutral aqueous solution (pH 6.0) than in a basic aqueous solution (pH 8.0), where the pH dependence of dissolution of the basic drug at the early stage of dissolution is reduced, and the ratio of the dissolution rate of the drug in the acidic test solution to the dissolution rate of the basic drug in the neutral test solution (dissolution rate in the acidic test solution/dissolution rate in the neutral test solution) decreases with dissolution time as the dissolution test proceeds (the ration being lower at the late stage than at the early stage of the dissolution test).

There are no particular limitations on the enteric polymer used in the invention, but it should dissolve in some aqueous buffer solutions at a pH anywhere in the range of 5.0 to 8.0 (in the range of 5.0 to 6.8; in the range of 5.0 to 6.0; or in the range of 5.0 to 5.5), although the enteric polymer does not dissolve in the 0.1 N hydrochloric acid solution. At least one enteric polymer can be used, or two or more enteric polymers may be mixed together. Exemplary enteric polymers include methacarylic acid copolymers, methacrylic acid-methyl methacrylate copolymers (EUDRAGIT® L100, EUDRAGIT® S100 and the like, Röhm GmbH, Germany), methacrylic acid-ethyl acrylate copolymers (EUDRAGIT® L100-55, EUDRAGIT® L30D-55, and the like, Röhm GmbH, Germany), hydroxypropyl methylcellulose phthalate (HP-55, HP-50, and the like, Shinetsu Chemical, Japan), hydroxypropyl methylcellulose acetate succinate (AQOAT®, Shinetsu Chemical, Japan), carboxymethyl ethylcellulose (CMEC, Freund Corporation, Japan), cellulose acetate phthalate and the like. Methacrylic acid-ethyl acrylate copolymers, methacrylic acid-methyl methacrylate copolymers, hydroxypropyl methylcellulose phthalate, hydroxypropyl methylcellulose acetate succinate, or mixtures of tow or more thereof are preferred. Methacrylic acid-ethyl acrylate copolymer, available as EUDRAGIT® L100-55, which is a water-dispersible enteric polymer powder, is particularly preferable. In one embodiment, the enteric polymer is methacrylic acid-ethyl acrylate copolymer, hydroxypropyl methylcellulose acetate succinate, or a mixture thereof. In another embodiment, the enteric polymer is hydroxypropyl methylcellulose acetate succinate (AQOAT® LF, AQOAT® MF, and the like, Shin-Etsu Chemical, Japan). There are no particular limitations on the mean particle size of the enteric polymer used in the invention, but generally the smaller the better, and the mean particle size may be from 0.05 to 100 μgm; from 0.05 to 70 μm; or from 0.05 to 50 μm.

The water-insoluble polymer refers to a sustained release base which does not dissolve in an aqueous buffer solution at a pH anywhere in the range of 1.0 to 8.0, and is not particularly limited. The matrix type sustained release formulations may comprise at least one water-insoluble polymer; or two or more water-insoluble polymers. Exemplary water-insoluble polymers include cellulose ethers (cellulose alkyl ethers, including cellulose C₁₋₆alkyl ethers, such as methylcellulose, ethylcellulose, propylcellulose, ethylmethylcellulose, ethylpropylcellulose, isopropylcellulose, butylcellulose and the like; cellulose aralkyl ethers such as benzyl cellulose and the like; cellulose cyanoalkyl ethers such as cyanoethyl cellulose, cyanomethyl cellulose, cyanoethylmethyl cellulose, cyanopropyl cellulose, and the like), cellulose esters (cellulose organic acid esters such as cellulose acetate butyrate, cellulose acetate, cellulose propionate, cellulose butyrate, cellulose acetate propionate and the like), methacrylic acid-acrylic acid copolymers (e.g., EUDRAGIT® RS, EUDRAGIT® RL, EUDRAGIT® NE, Röhm GmbH, Germany) and the like. Of these polymers, cellulose C₁₋₆alkyl ethers, aminoalkyl methacrylate copolymers (e.g., EUDRAGIT® RL, EUDRAGIT® RS, Röhm GmbH, Germany) and ethyl acrylate-methyl methacrylate copolymers (e.g., EUDRAGIT® NE, Röhm GmbH, Germany) are preferred. In one embodiment, ethylcellulose (ETHOCEL®, Dow Chemical) is preferred. There are no particular limitations on the mean particle size of the water-insoluble polymer, but generally the smaller the better. The mean particle size may be from 0.1 to 100 μm; from 1 to 50 μm; from 3 to 15 μm; or from 5 to 15 μm.

The amount of enteric polymer in the matrix type sustained release formulations is not particularly limited, but may be from 5 to 90% by weight; from 8 to 70% by weight; from 10 to 60% by weight; or from 15 to 50% by weight, based on 100% by weight of the matrix type sustained release formulation. In still other embodiments, the amount of enteric polymer in the matrix type sustained release formulations may be from 20 to 60% by weight; from 20 to 40% by weight; or from 20 to 30% by weight, based on 100% by weight of the matrix type sustained release formulation. In still other embodiments, the amount of enteric polymer in the matrix type sustained release formulations may be from 5% to 30% by weight; from 10% to 25% by weight; from 10% to 20% by weight; or from 15% to 20% by weight, based on 100% by weight of the matrix type sustained release formulation.

The amount of water-insoluble polymer in the matrix type sustained release formulations is not particularly limited, but may be from 1 to 90% by weight; from 3 to 70% by weight; from 5 to 50% by weight; or from 5 to 35% by weight, based on 100% by weight of the matrix type sustained release formulation. In other embodiments, the amount of water-insoluble polymer in the matrix type sustained release formulations may be from 10 to 15% by weight based on 100% by weight of the matrix type sustained release formulation. In still other embodiments, the amount of water-insoluble polymer in the matrix type sustained release formulations may be from 10% to 40% by weight; from 15% to 35% by weight; or from 20 to 30% by weight, based on 100% by weight of the matrix type sustained release formulation.

The amount of water-insoluble polymer and enteric polymer in the matrix type sustained release formulations is not particularly limited, but may be from 25% to 95% by weight; from 35% to 95% by weight; from 35% to 90% by weight, or from 35% to 75% by weight, based on 100% by weight of the matrix type sustained release formulation. In other embodiments, the amount of water-insoluble polymer and enteric polymer in the matrix type sustained release formulations may be from 30% to 80% by weight; from 40% to 70% by weight; or from 45% to 65% by weight, based on 100% by weight of the matrix type sustained release formulation. In other embodiments, the amount of water-insoluble polymer and enteric polymer in the matrix type sustained release formulations may be from 30% to 60% by weight; from 35% to 50% by weight; or from 40% to 45% by weight, based on 100% by weight of the matrix type sustained release formulation.

In one embodiment, the matrix type sustained release formulations of the invention, the enteric polymer may be a methacrylic acid-ethyl acrylate copolymer and/or hydroxypropyl methylcellulose acetate succinate, and the water-insoluble polymer may be ethylcellulose. In another embodiment, the enteric polymer may be a methacrylic acid-ethyl acrylate copolymer, a methacrylic acid-ethyl methacrylate copolymer, hydroxypropyl methylcellulose acetate succinate, or a mixture of two or more thereof, and the water-insoluble polymer may be ethylcellulose.

The matrix type sustained release formulations of the invention provide remarkable features, such that dissolution with low pH dependence of the basic drug at the early stage of dissolution can be ensured in the dissolution test and that, as the dissolution test proceeds, the ratio of the dissolution rate of the basic drug in an acidic dissolution test solution (hereafter “an acidic test solution”) to the dissolution rate of the basic drug in a neutral dissolution test solution (hereafter “a neutral test solution”) (dissolution rate in the acidic test solution/dissolution rate in the neutral test solution) decreases with dissolution time at the late stage of dissolution, as compared to the early stage of dissolution. In the matrix type sustained release formulations of the invention, by mixing the enteric polymer with the basic drug having higher solubility in the acidic aqueous solution and the neutral aqueous solution than in the basic aqueous solution described above, the dissolution of the basic drug can be inhibited in the acidic and neutral dissolution test solutions. When mixing with water-insoluble polymer and enteric polymer, the greater the amount of enteric polymer mixed with the water-insoluble polymer the greater the reduction in dissolution speed of the basic drug in the acidic and neutral dissolution test solutions, thus easily providing matrix type sustained release formulations wherein dissolution with low pH dependence of the basic drug at the early stage of dissolution can be ensured in the dissolution test, and wherein, as the dissolution test proceeds, the ratio of the dissolution rate of the basic drug in the acidic test solution to the dissolution rate of the basic drug in the neutral test solution (dissolution rate in the acidic test solution/dissolution rate in the neutral test solution) decrease with dissolution time (specifically the ratio decreases at the late stage of dissolution, as compared to the early stage of dissolution).

The characteristic features of the matrix type sustained release formulations of the invention can be demonstrated by dissolution profile in a 50 mM phosphate buffer (pH 6.8) as the neutral dissolution test solution and in 0.1 N hydrochloric acid solution as the acidic dissolution test in the dissolution test. When the basic drug is released from the matrix type sustained release formulation of the invention in a dissolution test according to the Japanese Pharmacopoeia (1 4th Edition) paddle method, the ratio of the dissolution rate in a 0.1 N hydrochloric acid solution to the dissolution rate of the basic drug in a 50 mM phosphate buffer (pH 6.8) decreases with dissolution time until a dissolution time at which the dissolution rate in a 50 mM phosphate buffer (pH 6.8) is 90%. Moreover, the invention provides matrix type sustained release formulations wherein, in the dissolution test according to the Japanese Pharmacopoeia (14th Edition) paddle method, the dissolution rate in the 0.1 N hydrochloric acid solution at a dissolution time of 1 hour is not more than 60%; not more than 50%; or not more than 40%. In the early stage of dissolution in the dissolution test according to the Japanese Pharmacopoeia (14th Edition) paddle method, the ratio of the dissolution rate in the 0.1 N hydrochloric acid solution to the dissolution rate in the 50 mM phosphate buffer (pH 6.8) is from 0.3 to 1.5; from 0.3 to 1.4; from 0.3 to 1.3; or from 0.3 to 1.2, at a dissolution time of 3 hours. The Japanese Pharmacopoeia (14th Edition) paddle method for dissolution tests is described in the Japanese Pharmacopoeia, 14th Edition, and, for example, the test can be performed at a paddle rate of 50 rpm.

The matrix type sustained release formulations of the invention may also comprise (i) one or more water-soluble sugars, (ii) one or more water-soluble sugar alcohols, or (iii) one or more water-soluble sugars and one or more water-soluble sugar alcohols. There are no particular limitations on the water-soluble sugars and/or water-soluble sugar alcohols. Exemplary water-soluble sugars include lactose, sucrose, glucose, dextrin, pullulan and the like. Exemplary water-soluble sugar alcohols include mannitol, erythritol, xylitol, sorbitol and the like. Lactose and mannitol may be used as the water-soluble sugar and water-soluble sugar alcohol, respectively. There are no particular limitations on the amount of water-soluble sugar and/or water-soluble sugar alcohol in the matrix type sustained release formulations, but the amount may be from 3% to 70% by weight; from 5% to 60% by weight; from 10% to 60% by weight; or from 12% to 60% by weight; based on 100% by weight of the matrix type sustained release formulation. In one embodiment, the amount of water-soluble sugar and/or water-soluble sugar alcohol in the matrix type sustained release formulation may be from 20% to 30% by weight. In another embodiment, the amount of water-soluble sugar and/or water-soluble sugar alcohol in the matrix type sustained release formulation may be from 35% to 55% by weight, or from 40% to 50% by weight.

The matrix type sustained release formulations of the invention may further comprise a variety of pharmaceutically acceptable excipients, such as diluents, lubricants, binders, disintegrators, preservatives, anti-oxidants, colorants, sweeteners, plasticizers, and the like. Exemplary diluents that may be used in the formulations include starch, pregelatinized starch, crystalline cellulose, light anhydrous silicic acid, synthetic aluminum silicate, magnesium aluminate metasilicate and the like. The amount of diluent in the formulations of the invention may be from 0 to 10% by weight. Exemplary lubricants include magnesium stearate, calcium stearate, talc, sodium stearyl fumarate and the like. The amount of lubricant in the formulations of the invention may be from 0 to 5% by weight; from 0.01% to 4% by weight; from 0.1% to 3% by weight; or from 0.3% to 1% by weight. Exemplary binders include hydroxypropylcellulose, methylcellulose, carboxymethylcellulose sodium, hydroxypropyl methylcellulose, polyvinylpyrrolidone and the like. The amount of binder may be 0 to 10% by weight; from 0.1 to 8% by weight; from 0.5 to 6% by weight; or from 1% to 3% by weight. Exemplary disintegrators include carboxymethyl cellulose, carboxymethyl cellulose calcium, croscarmellose sodium, carboxymethyl starch sodium, low-substituted hydroxypropylcellulose and the like. The amount of disintegrator may be 0 to 5% by weight. Exemplary preservatives include paraoxybenzoic acid esters, chlorobutanol, benzyl alcohol, phenethyl alcohol, dehydroacetic acid, sorbic acid and the like. The amount of preservative may be 0 to 5% by weight. Exemplary anti-oxidants include sulfites, ascorbates and the like. The amount of anti-oxidant may be 0 to 5% by weight. Exemplary colorants include non-water-soluble lake pigments, natural pigments (such as β-carotene, chlorophyll and iron oxide), yellow ferric oxide, red ferric oxide, yellow iron sesquioxide, red iron sesquioxide, black iron oxide and the like. The amount of colorant may be from 0 to 8% by weight. Exemplary sweeteners include sodium saccharin, dipotassium glycyrrhizate, aspartame, stevia and the like. The amount of sweetener may be from 0 to 10% by weight. Exemplary plasticizers include glycerin fatty acid esters (e.g., MYVACET®), triethyl citrate (e.g., CITROFLEX® 2), propylene glycol, polyethylene glycol and the like. The amount of plasticizer may be 0 to 10% by weight. The matrix type sustained release formulations may also have an outer film coating. Exemplary film coating bases include hydroxypropyl methylcellulose, hydroxypropyl cellulose and the like.

The matrix type sustained release formulations of the invention can be manufactured by methods comprising the steps of: mixing a basic drug which has higher solubility in a 0.1 N hydrochloric solution and a neutral aqueous solution (e.g., 50 mM phosphate buffer) (pH 6.0) than in a basic aqueous solution (e.g., 50 mM phosphate buffer) (pH 8.0) with at least one enteric polymer; and compression-molding the resulting mixture. The methods may further comprise mixing the basic drug with at least one enteric polymer and at least one water-insoluble polymer. One or more water-soluble sugars and/or water-soluble sugar alcohols and other pharmaceutically acceptable excipients may also be used in the matrix type sustained release formulations as necessary. Alternatively, the matrix type sustained release formulations of the invention can also be manufactured by the steps of mixing (1) at least one basic drug which has a solubility in a 0.1 N hydrochloric acid solution and a neutral aqueous solution (pH 6.0) of 1 mg/ml or more; a solubility in a basic aqueous solution (pH 8.0) of 0.2 mg/ml or less; wherein the solubility in the neutral aqueous solution (pH 6.8) is at least twice its solubility in the basic aqueous solution (pH 8.0), and the solubility is not more than half its solubility in the neutral aqueous solution (pH 6.0), with (2) at least one enteric polymer; and compression molding the resulting mixture. The methods may further comprise mixing the basic drug with at least one enteric polymer and at least one water-insoluble polymer. One or more water-soluble sugars and/or water-soluble sugar alcohols and other pharmaceutically acceptable excipients may also be used in the matrix type sustained release formulations as necessary. Mixing and compression-molding are accomplished by the ordinary methods commonly used in the formulation field. The matrix type sustained release formulations can be manufactured by the direct method of compression-molding using a tabletting machine after the mixing step. The matrix type sustained release formulations can also be manufactured by methods which comprise the step of granulating the mixture after mixing and before compression-molding. For example, any granulating methods can be used including wet granulation methods, dry granulation methods, fluidized bed granulation methods, wet screening methods, spray-drying methods and the like.

The matrix type sustained release formulations are not particularly limited as long as they are an oral preparation. For example, tablets, granules (e.g., coarse granules, fine granules), capsules and the like can be manufactured. Capsules can be packed with 1 or more tablets, and/or granules (e.g., fine granules, coarse granules). For example, hard capsules can be packed with a plurality of small-diameter mini-tablets, or with granules (e.g., coarse granules, fine granules), or with both tablets and granules (e.g., coarse granules fine granules). The matrix type sustained release formulations can also be given a film coating as necessary. It should be noted that the presence or absence of a water-soluble film coating has very little effect on the dissolution profile of the basic drug from the matrix type sustained release formulations.

The invention also provides methods of reducing the pH dependence of dissolution of a basic drug at a dissolution time of 2 to 3 hours (corresponding to gastric emptying time) at the early stage of the dissolution test by mixing the basic drug (the solubility of which is higher in a 0.1 N hydrochloric solution and a neutral aqueous solution (e.g., 50 mM phosphate buffer) (pH 6.0) than in a basic aqueous solution (e.g., 50 mM phosphate buffer) (pH 8.0) with at least one enteric polymer and, optionally, at least one water-insoluble polymer, and then compression-molding the mixture.

The invention also provides methods for controlling release of a basic drug with low pH dependence comprising the steps of mixing (1) a basic drug which has solubility in a 0.1 N hydrochloric acid solution and a 50 mM phosphate buffer (pH 6.0) of 1 mg/ml or more; a solubility in a 50 mM phosphate buffer (pH 8.0) of 0.2 mg/ml or less; and which has solubility in a 50 mM phosphate buffer (pH 6.8) of at least twice its solubility in a 50 mM phosphate buffer (pH 8.0) and is not more than half its solubility in a 50 mM phosphate buffer (pH 6.0), with (2) at least one enteric polymer and (3) at least one water-insoluble polymer; and compression molding the resulting mixture.

The matrix type sustained release formulations of the invention can be manufactured by, for example, the following methods. 130 grams of donepezil hydrochloride (Eisai Co., Ltd.), 624 grams of ETHOCEL® 10FP (ethylcellulose, Dow Chemical), 780 grams of EUDRAGIT® L100-55 (Röhm GmbH, Germany) and 988 grams of lactose will be mixed in a granulator. Wet granulation will be accomplished by adding an aqueous solution of 52 grams of hydroxypropyl cellulose dissolved in a suitable amount of purified water, and the resulting grains will be heat-dried using a tray dryer, and sieved to obtain the desired granule size. After sieving, 1 gram of magnesium stearate based on 99 grams of the granule will be added and mixed, and a rotary tabletting machine will then be used to obtain tablets with 8 mm diameters containing 10 mg of donepezil hydrochloride in a 200 mg tablet. A coating machine can also be used to coat these tablets with a water-soluble film containing hydroxypropyl methylcellulose or the like as its main component.

The matrix type sustained release preparation according to the present invention can also be manufactured by, for example, the following methods. 20 g of memantine hydrochloride (Lachema s.r.o., Czech Republic), 48 g of ETHOCEL® 10FP (ethylcellulose, Dow Chemical), 60 g of EUDRAGIT® L100-55 and 66 g of lactose will be mixed in a granulator. Wet granulation will be accomplished by adding an aqueous solution of 4 g of hydroxypropyl cellulose dissolved in a suitable amount of purified water, and the resulting grains will be heat-dried using a tray dryer, and sieved to the desired granule size. After sieving, 1 g of magnesium stearate based on 99 g of granule will be added and mixed, and a rotary tabletting machine will then be used to obtain tablets with 8 mm diameters containing 20 mg of memantine hydrochloride based on 200 mg of the granule. A coating machine can also be used to coat these tablets with a water-soluble film containing hydroxypropyl methylcellulose or the like as its main component.

The examples described herein are for purposes of illustration only, and are not intended to limit the scope of the appended claims.

### EXPERIMENTAL EXAMPLE 1

This examples shows the dissolution effects of an enteric polymer mixed with a water-insoluble polymer in the matrix type sustained release formulations of the invention.

Matrix type sustained release formulations were prepared using donepezil hydrochloride according to Comparative Example 1, and Examples 2 and 4 which are given below, and dissolution tests were performed thereon. The matrix type sustained release formulations were prepared using ethylcellulose as the water-insoluble polymer and EUDRAGIT® L100-55 as the enteric polymer. The ratios of ethylcellulose to EUDRAGIT® LI00-55 in Comparative Example 1, and Examples 2 and 4 were 25%:0% by weight, 25%:25% by weight and 25%: 50% by weight, respectively.

The dissolution tests were performed in test solutions A and B at a paddle frequency of 50 rpm in accordance with the dissolution test methods of the Japanese Pharmacopoeia, 14th Ed. Test solution A was a 0.1 N hydrochloric acid solution. Test solution B was a 50 mM phosphate buffer, pH 6.8 (i.e., buffer of 50 mM sodium phosphate solution with pH adjusted with hydrochloric acid to be from 6.75 to 6.84).

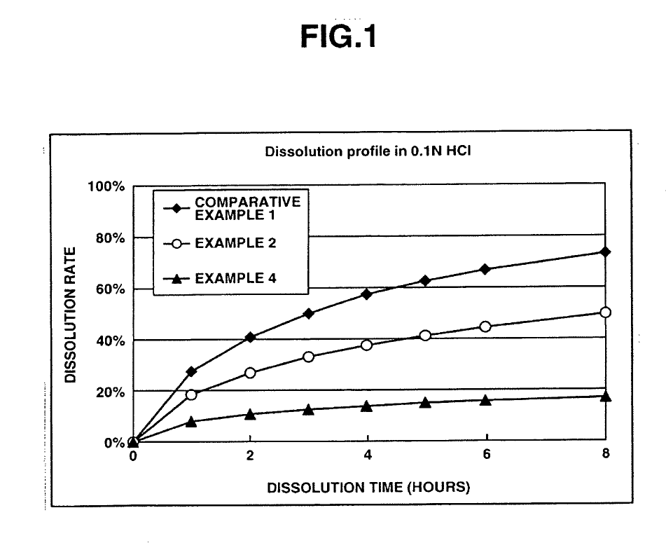

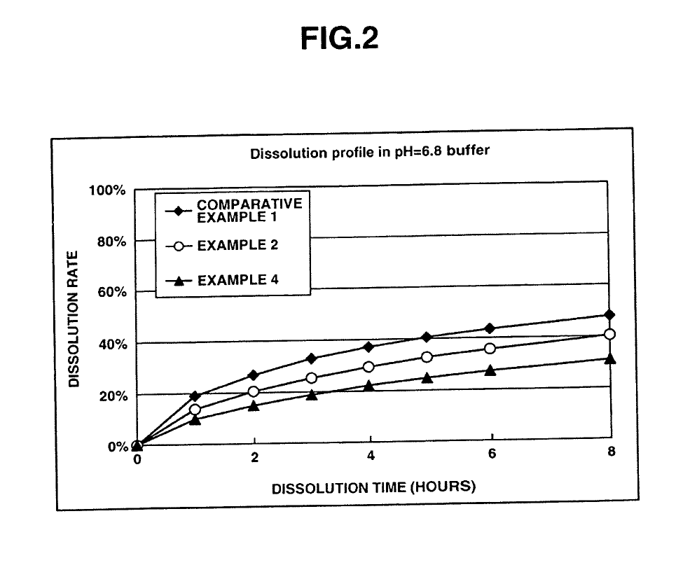

The dissolution rate was calculated from concentrations of donepezil hydrochloride in sample solutions collected with dissolution time and analyzed by a spectrophotometric method or HPLC analysis method. The spectrophotometric method was performed under measurement conditions of a wavelength at 315 nm, and a reference wavelength at 650 nm. HPLC analysis method was performed under measurement conditions of measurement column: Inertsil ODS-2 (GL Science), mobile phase: water/acetonitrile/70% aqueous perchloric acid solution=650/350/1 mixture, and detection wavelength at 271 nm. Comparative results of the dissolution tests are shown in FIGS. 1 and 2. Results for Comparative Example 1 and Examples 2 and 4 are shown in Tables 1 and 2.

In Comparative Example 1, and Examples 2 and 4, the weight percentage of EUDRAGIT® L100-55 in the tablets varied under a constant ratio of 25% by weight of ethylcellulose per 100% by weight of the tablets (0%, 25%, 50% by weight per 100% by weight of EUDRAGIT® L100-55 per 100% by weight of the tablets, respectively). As shown in FIGS. 1 and 2, it was confirmed that in dissolution tests using the same test solutions under the same conditions, the greater the content (content percentage) of the enteric polymer (EUDRAGIT® L100-55), the slower the dissolution of donepezil hydrochloride from the matrix sustained release formulation of the invention.

In the matrix sustained release formulation of the invention, when both the enteric polymer and the water-insoluble polymer were mixed into the formulation, the greater the amount of the enteric polymer mixed with the water-insoluble polymer, the more the dissolution speed was reduced, thus providing a long-acting sustained-release formulation.

### EXPERIMENTAL EXAMPLE 2

Set out below are the effects of ensuring dissolution with low pH dependence in the matrix type sustained release formulation, at the same time, of reducing the ratio of dissolution rate of the basic drug in an acidic test solution to the dissolution rate in a neutral test solution (dissolution rate in the acidic test solution/dissolution rate in the neutral test solution) in a dissolution test, as the dissolution tests proceeded.

EUDRAGIT® L100-55 was used as the enteric polymer and ethylcellulose was used as the water insoluble polymer in the matrix sustained-release preparation.

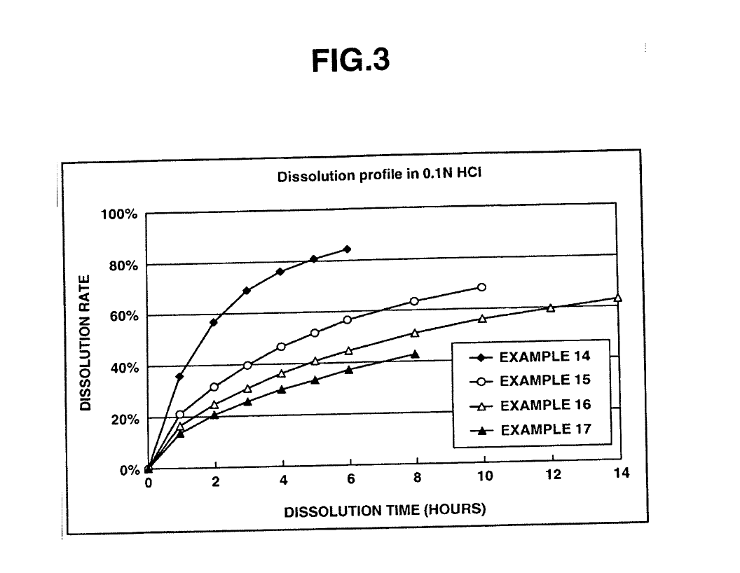

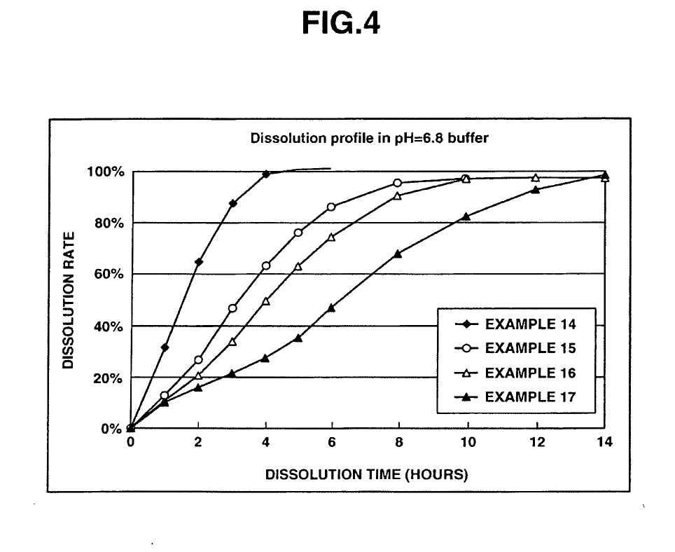

Matrix type sustained release formulations were prepared using donepezil hydrochloride according to Comparative Example 1, and Examples 1-11 and 14-17 below, and dissolution tests were performed thereon. The dissolution tests were performed to evaluate formulations in which the amounts of donepezil hydrochloride, the enteric polymer and the water-insoluble polymer varied (Examples 1-6), in which the type of excipients varied (Examples 5 and 7), in which wet granulation was performed using a binder (Examples 8, 11 and 14-17), in which the type of ethylcellulose varied (Examples 5, 9 and 10) and in which scale-up production was carried out (Examples 11, and 14-17). A preparation containing donepezil hydrochloride and the water-insoluble polymer as its main components without any enteric polymer was used as Comparative Example 1. The results for Comparative Example 1 and Examples 1, 2-6, 7-11 and 14-17 are shown in Tables 1, 2, 3 and 4, respectively. Comparative dissolution test results for Examples 14 to 17 are shown in FIGS. 3 and 4.

In Comparative Example 1, which did not contain an enteric polymer, the ratio of dissolution rate in the acidic test solution to the dissolution rate in the neutral test solution (dissolution rate in the acidic test solution/dissolution rate in the neutral test solution) increased slightly from a dissolution time of 1 hour to a dissolution time of 2 to 3 hours at the early stage, and the ratio subsequently remained at 1.5 with little change therein at the late stage of dissolution. On the other hand, in the inventive examples (i.e., Examples 1-11, and Examples 14-17) which contained an enteric polymer (i.e., EUDRAGIT® L100-55), the ratio of the dissolution rate decreased from a dissolution time of 1 hour to a dissolution time of 2 to 3 hours, and continued to decrease gradually as the dissolution test proceeded, until completion of the dissolution test or until a dissolution time at which the dissolution rate in the neutral test solution was 90% or more. The ratio of dissolution rates was from 0.6 to 1.3 at a dissolution time of 3 hours in these cases. Using an enteric polymer in the formulation of the invention provides a dissolution rate in an acidic test solution that is inhibited at the early stage of dissolution (corresponding to the gastric retention period) while reducing pH dependence of the basic drug, and a higher dissolution rate in the neutral test solution relative to the dissolution rate in the acidic test solution can be achieved at the late stage of dissolution (which is thought to correspond to the small intestinal retention stage). The effects at the early and late stages of dissolution were confirmed with all the formulations in which the amount of donepezil hydrochloride, enteric polymer and water-insoluble polymer varied (i.e., Examples 1-6), in which the type of diluents varied (i.e., Examples 5 and 7), in which wet granulation was achieved with a binder (i.e., Examples 8, 11, 14-17 and 20), in which the type of ethylcellulose varied (i.e., Examples 5, 9, and 10) and in which the manufacturing scale was altered (i.e., Examples 11, 14-17 and 20). In Examples 11 and 14-16, in particular, because 90% or more of the drug was released in the 50 mM phosphate buffer, pH 6.8, within 8 hours (which is estimated as the upper limit of large intestinal transit time in humans (Int. J Pharm., 53:107-117 (1989)), there is little risk of decreased bioavailability due to the sustained release characteristics, such that the formulations would be extremely useful.

### EXPERIMENTAL EXAMPLE 3

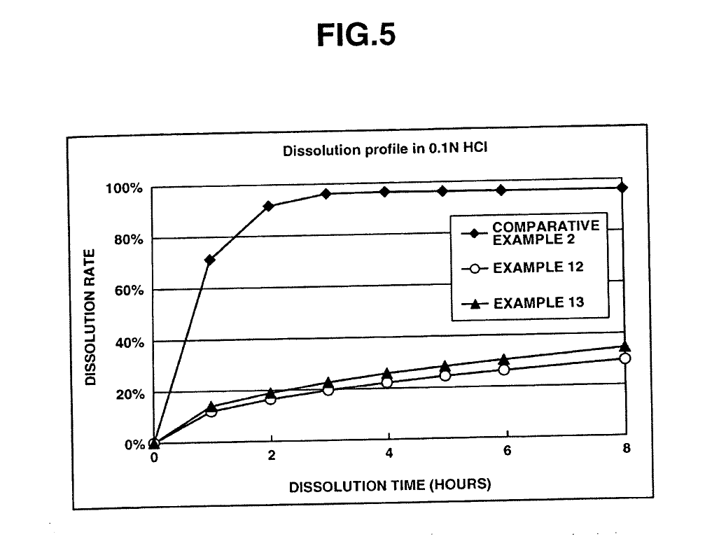

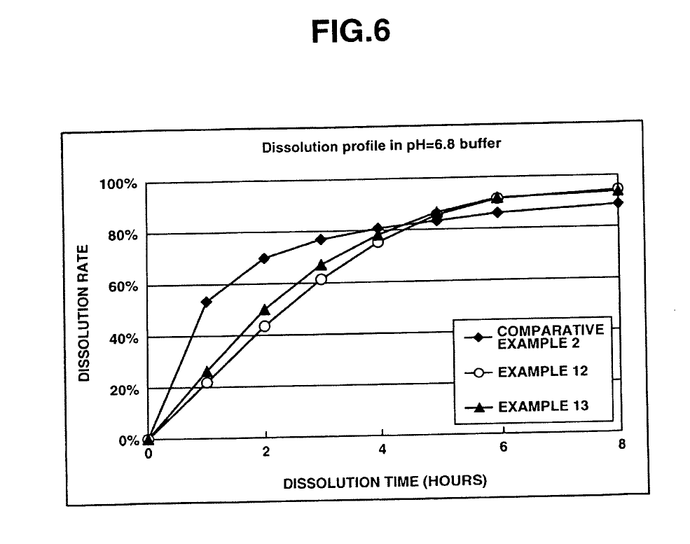

In this experimental examples, the types of enteric polymer and water insoluble polymer were evaluated for the matrix type sustained release formulation. The following experimental examples of the formulation of the invention use hydroxypropyl methylcellulose acetate succinate as the enteric polymer and ethylcellulose as the water-insoluble polymer. The formulations were prepared using donepezil hydrochloride according to Comparative Example 2, and Examples 12 and 13 which are given below, and dissolution tests were performed thereon. Hydroxypropyl methylcellulose acetate succinate (AQOAT® LF or AQOAT® MF, Shin-Etsu Chemical, Japan) was used as the enteric polymer and ethylcellulose was used as the water-insoluble polymer. The amount of hydroxypropyl methylcellulose acetate succinate in the preparations was 50% based on the total weight of the formulation. A formulation containing the same amount of donepezil hydrochloride and water-insoluble polymer as in Examples 12 and 13 but no enteric polymer was used as Comparative Example 2. Comparative results of the dissolution tests are shown in FIGS. 5 and 6, and results for Comparative Example 2, and Examples 12 and 13 are shown in Table 5.

As shown in FIGS. 5 and 6, there was a dramatically slower dissolution of donepezil hydrochloride in the acidic test solution as a result of using 50% enteric polymer in the formulation. The greater the amount of enteric polymer mixed with the water-insoluble polymer, the more dissolution speed can be retarded, thus allowing for the production of a sustained release formulation.

In Comparative Example 2, which did not contain an enteric polymer, the dissolution rate in the acidic test solution reached 90% at a dissolution time of 2 hours, and the ratio of dissolution rate in the acidic test solution to dissolution rate in the neutral test solution (dissolution rate in the acidic test solution/dissolution rate in the neutral test solution) remained roughly constant at 1.3 at the early stage of dissolution (i.e., 1 to 3 hours), while in Examples 12 and 13, which used 50.0% hydroxypropyl methylcellulose acetate succinate (AQOAT® LF or AQOAT® MF, Shin-Etsu Chemical, Japan) as the enteric polymer, the ratio of the dissolution rate was 0.38 to 0.55, which was lower than in Comparative Example 2. The enteric polymer retarded the dissolution rate of the drug in the acidic and neutral test solutions at the early stage of dissolution and, in particular, dramatically retarded the dissolution rate of the drug in the acidic test solution, thus bringing the dissolution rates in the two solutions closer to each other and reducing pH dependence. Moreover, at the late stage of dissolution it also retarded dissolution in the acidic test solution while increasing dissolution in the neutral test solution. This suggests that the risk of adverse events at the early stage of dissolution can be reduced, and the risk of reduced bioavailability can be inhibited in these formulations. Accordingly, by setting the added amount of hydroxypropyl cellulose acetate succinate (AQOAT® LF or AQOAT® MF, Shin-Etsu Chemical, Japan) to an appropriate value between 0 and 50%, it is possible to design a formulation in which dissolution behavior with the ratio of dissolution rate in the acidic test solution to dissolution rate in the neutral test solution (dissolution rate in the acidic test solution/dissolution rate in the neutral test solution) being near 1 at the early stage of dissolution can be ensured and in which this ratio of dissolution rates can be decreased until the dissolution rate in a neutral solution reaches 90% or more at the late stage of dissolution.

Table 6 shows that effects of a combination of ethylcellulose and EUDRAGIT® L100 on dissolution behavior of the formulation. As compared to Comparative Example 2 which contains 25% ethylcellulose, it was confirmed in Example 21, which contains 25% ethylcellulose and 50% EUDRAGIT® L100, that a ratio of dissolution rate of the basic drug in the acidic test solution to dissolution rate of the basic drug in the neutral test solution (dissolution rate in the acidic test solution/dissolution rate in the neutral test solution) decreased with dissolution time.

Moreover, effects of EUDRAGIT® RSPO as the water-insoluble polymer on the dissolution behavior of the formulation were also evaluated as follows. As shown in Table 7, Comparative Example 3, which does not contain enteric polymer but does contain EUDRAGIT® RSPO, exhibited a drug burst behavior and no effect on the sustained-release characteristics of the basic drug. Examples 22 and 23, which contained EUDRAGIT® L100 and AQOAT® LF, respectively, showed that the dissolution time was prolonged in both test solution A and test solution B, and the effect of the sustained-release characteristics was accomplished by mixing the enteric polymer into the formulations in these examples. In Examples 22 and 23, a ratio of the dissolution rate of the basic drug in the acidic test solution to dissolution rate of the drug in the neutral test solution (dissolution rate in the acidic test solution/dissolution rate in the neutral test solution) exhibited 0.34 and 0.7 at the dissolution time of 3 hours, respectively. It was confirmed that the above ratio of the dissolution rate of the basic drug in the acidic solution to dissolution rate of the basic drug in the neutral solution decreased with dissolution time, after dissolution time of 3 hours. Example 23 showed that 90% or more of the basic drug was released in the 50 mM phosphate buffer (pH 6.8) within 8 hours, which is estimated as the upper limit of large intestinal transit time in humans, such that the formulation of Example 23 should be extremely useful.

### EXPERIMENTAL EXAMPLE 4

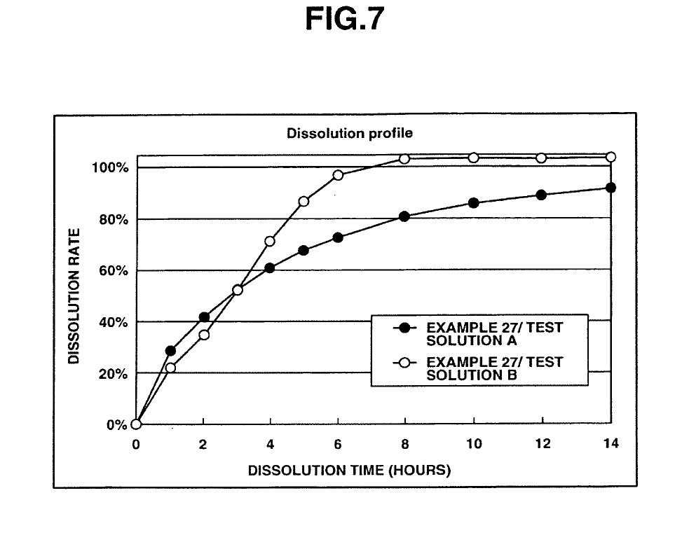

The dissolution tests were carried out using tablets prepared in Examples 27-31. The results of the dissolution tests are shown in Table 8 and in FIGS. 7-9. In the invention, it is evident that the matrix type sustained-release formulation has properties that ensure dissolution with low pH dependence of the basic drug at the early stage of dissolution and that allow the ratio of dissolution rate of the basic drug in the acidic test solution to the dissolution rate of the basic drug in the neutral test solution (dissolution rate in the acidic test solution/dissolution rate in the neutral test solution) to be decreased at the late stage of dissolution, as the dissolution test proceeds. These formulations exhibited that 90% or more of the basic drug was released in the 50 mM phosphate buffer (pH 6.8) within 8 hours, which is estimated as the upper limit of large intestinal transit time in humans, such that the formulations of the invention in Examples 25 and 26 should be extremely useful.

### EXPERIMENTAL EXAMPLE 5

In order to evaluate dissolution behavior of pharmaceutical compositions containing memantine hydrochloride, dissolution tests were carried out using the formulations obtained in the following Examples and Comparative Examples. The dissolution tests were performed in the following two types of test solutions at a paddle frequency of 50 rpm in accordance with the dissolution test methods of the Japanese Pharmacopoeia, 14th Edition. The dissolution tests were carried out using test solution A as the acidic test solution and test solution B as the neutral test solution. Test solution A was a 0.1 N hydrochloric acid solution. Test solution B was a 50 mM phosphate buffer, pH 6.8 (i.e., a buffer of 50 mM sodium phosphate solution with a pH adjusted with hydrochloric acid to be from pH 6.75 to pH 6.84).

The dissolution rate was calculated from concentrations of memantine hydrochloride in sample solutions collected with dissolution time and analyzed by an HPLC method after memantine hydrochloride was fluorescently labeled with Fluorescamine. The conditions for fluorescence labeling and HPLC analysis are as follows. After sample solutions (1 ml) collected with dissolution time were mixed with borate buffer, pH 9.0 (USP), an acetone solution (5 ml) containing Fluorescamine (1.2 mg/ml) was added and stirred. Water (10 ml) was also added into the above solution and mixed to obtain a test sample. The test sample was analyzed by HPLC. HPLC analysis was performed under measurement conditions of measurement column: CAPCELL PAK UG120 C18 (Shiseido) or a similar column, column temperature: 40° C.; mobile phase: borate buffer, pH 9.0 (USP)/acetonitrile=60/40 mixture, and detection conditions: fluorescence detector (excitation wavelength/detection wavelength=391 nm/474 nm) at 271 nm.

The dissolution test were performed using tablets obtained in Examples 40-42 and Comparative Example 4 in order to evaluate effects of an enteric polymer on the formulations containing memantine hydrochloride and ethylcellulose as the water-insoluble polymer.

Comparative Example 4, which does not contain an enteric polymer but does contain EUDRAGIT® RSPO, showed that the dissolution rate of memantine hydrochloride was inhibited to be from 30 to 40% at the dissolution time of 1 hour. The ratio of dissolution rate of the drug in the acidic test solution to dissolution rate of the basic drug in the neutral test solution (dissolution rate in the acidic test solution/dissolution rate in the neutral test solution) was constant without change in the dissolution time. Example 40-42, which contain the enteric polymer, showed that the dissolution rate of memantine hydrochloride at the early stage of the dissolution was much lower than that in Comparative Example 4, and it was confirmed that the dissolution rate of memantine hydrochloride could be inhibited at the early stage of dissolution. Moreover, it was confirmed in these Examples that the ratio of dissolution rate of the basic drug in the acidic test solution to dissolution rate of the basic drug in the neutral test solution (dissolution rate in the acidic test solution/dissolution rate in the neutral test solution) decreased with the dissolution time.

Dissolution tests were performed using tablets obtained in Example 43 and Comparative Example 5 in order to evaluate effects of an enteric polymer on dissolution behavior of the formulations containing memantine hydrochloride and EUDRAGIT® RSPO as the water insoluble polymer.

Comparative Example 5, which does not contain the enteric polymer but does contain EUDRAGIT® RSPO, showed that a dissolution rate of memantine hydrochloride in the acidic and the neutral test solutions was not less than 90%, a ratio of dissolution rate of the basic drug in the acidic test solution to dissolution rate of the basic drug in the neutral test solution (dissolution rate in the acidic test solution/dissolution rate in the neutral test solution) was constant without change in the dissolution time.

Example 43, which contains the enteric polymer, showed that the dissolution rate of memantine hydrochloride at the early stage of the dissolution was much lower than that in Comparative Example 5, and it was confirmed that the dissolution rate of memantine hydrochloride could be inhibited at the early stage of dissolution. Moreover, it was confirmed in Example 43 that the ratio of dissolution rate of the basic drug in the acidic test solution to dissolution rate of the basic drug in the neutral test solution (dissolution rate in the acidic test solution/dissolution rate in the neutral test solution) decreased with the dissolution time.

### EXAMPLE 1

300 mg of donepezil hydrochloride (Eisai Co. Ltd.), 1500 mg of EUDRAGIT® L100-55 (Röhm GmbH, Germany), 1170 mg of lactose and 30 mg of magnesium stearate (Mallinckrodt Baker, Inc.) were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 1.

### EXAMPLE 2

300 mg of donepezil hydrochloride (Eisai Co. Ltd.), 750 mg of ETHOCEL® 10FP (ethylcellulose, Dow Chemical), 750 mg of EUDRAGIT® L100-55, 1170 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 2.

### EXAMPLE 3

75 mg of donepezil hydrochloride (Eisai Co. Ltd.), 750 mg of ETHOCEL® 10FP, 750 mg of EUDRAGIT® L100-55, 1395 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 5 mg of donepezil hydrochloride. The results of the dissolution test are shown in Table 2.

### EXAMPLE 4

300 mg of donepezil hydrochloride (Eisai Co. Ltd.), 750 mg of ETHOCEL® 10FP, 1500 mg of EUDRAGIT® L100-55, 420 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 2.

### EXAMPLE 5

300 mg of donepezil hydrochloride (Eisai Co. Ltd.), 375 mg of ETHOCEL® 10FP (ethylcellulose, Dow Chemical), 1500 mg of EUDRAGIT® L100-55, 795 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 2.

### EXAMPLE 6

300 mg of donepezil hydrochloride (Eisai Co. Ltd.), 183 mg of ETHOCEL® 10FP, 1500 mg of EUDRAGIT® L100-55, 987 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 2.

### EXAMPLE 7

300 mg of donepezil hydrochloride (Eisai Co. Ltd.), 375 mg of ETHOCEL® 10FP, 1500 mg of EUDRAGIT® L100-55, 795 mg of D-mannitol and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 3.

### EXAMPLE 8

A suitable amount of purified water was added to and mixed with 300 mg of donepezil hydrochloride (Eisai Co. Ltd.), 375 mg of ETHOCEL® 10FP, 1500 mg of EUDRAGIT® L100-55, 705 mg of lactose and 90 mg of hydroxypropyl cellulose (HPC-L, Nippon Soda Co., Ltd.), and the mixture was heat-dried in a hydrostatic chamber. 30 mg of magnesium stearate was added to and mixed with the dried granules. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 3.

### EXAMPLE 9

300 mg of donepezil hydrochloride (Eisai Co. Ltd.), 375 mg of ETHOCEL® 10STD, 1500 mg of EUDRAGIT® L100-55, 795 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 3.

### EXAMPLE 10

300 mg of donepezil hydrochloride (Eisai Co. Ltd.), 375 mg of ETHOCEL® 10FP, 1500 mg of EUDRAGIT® L100-55, 795 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 3.

### EXAMPLE 11

70 g of donepezil hydrochloride, 336 g of ETHOCEL® 10FP, 364 g of EUDRAGIT® L100-55, and 588 g of lactose were mixed. Wet granulation was carried out by adding an aqueous solution of 28 g of hydroxypropyl cellulose (HPC-L, Nippon Soda Co., Ltd.) dissolved in a suitable amount of purified water to this mixture. The resulting granules were heat-dried in a tray dryer, and sieved to obtain the desired granule size. After sieving, 1 g of magnesium stearate based on 99 g of granules was added and mixed. A rotary tabletting machine was used to make the granules into tablets with 8 mm diameters containing 10 mg of donepezil hydrochloride in a 200 mg tablet. The results of the dissolution test are shown in Table 3.

### EXAMPLE 12

300 mg of donepezil hydrochloride, 375 mg of ETHOCEL® 10FP, 1500 mg of AQOAT® LF (hydroxypropyl methylcellulose acetate succinate, Shinetsu Chemical), 795 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 5.

### EXAMPLE 13

300 mg of donepezil hydrochloride (Eisai Co. Ltd.), 375 mg of ETHOCEL® 10FP (ethylcellulose, Dow Chemical), 1500 mg of AQOAT® MF (hydroxypropyl methylcellulose acetate succinate, Shinetsu Chemical), 795 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 5.

### EXAMPLE 14

130 g of donepezil hydrochloride (Eisai Co. Ltd.), 312 g of ETHOCEL® 10FP (ethylcellulose, Dow Chemical), 624 g of EUDRAGIT® L100-55 (Rohm Pharma) and 1456 g of lactose were mixed in a granulator. Wet granulation was carried out by adding an aqueous solution of 52 g of hydroxypropyl cellulose dissolved in a suitable amount of purified water to this mixture. The resulting granules were heat-dried in a tray dryer, and sieved to obtain the desired granule size. After sieving, 1 g of magnesium stearate based on 99 g of granules was added and mixed, and a rotary tabletting machine was used to make tablets with 8 mm diameters containing 10 mg of donepezil hydrochloride in a 200 mg tablet. Opadry yellow (Colorcon Japan Limited) was used to give the resulting tablets a water-soluble coating containing hydroxypropyl methylcellulose as its main component (coating amount: 8 mg/tablet) to obtain film-coated tablets. The results of the dissolution test are shown in Table 4.

### EXAMPLE 15

130 g of donepezil hydrochloride (Eisai Co., Ltd.), 624 g of ETHOCEL® 10FP (ethylcellulose, Dow Chemical), 780 g of EUDRAGIT® L100-55 (Rohm Pharma) and 988 g of lactose were mixed in a granulator. Wet granulation was carried out by adding an aqueous solution of 52 g of hydroxypropyl cellulose dissolved in a suitable amount of purified water to this mixture. The resulting granules were heat-dried in a tray dryer, and sieved to obtain the desired granule size. After sieving, 1 g of magnesium stearate based on 99 g of granules was added and mixed, and a rotary tabletting machine was used to make tablets with 8 mm diameters containing 10 mg of donepezil hydrochloride in a 200 mg tablet. Opadry yellow (Colorcon Japan Limited) was used to give the resulting tablets a water-soluble coating containing hydroxypropyl methylcellulose as its main component (coating amount: 8 mg/tablet) to obtain film-coated tablets. The results of the dissolution test are shown in Table 4.

### EXAMPLE 16

130 g of donepezil hydrochloride (Eisai Co. Ltd), 780 g of ETHOCEL® 10FP (ethylcellulose, Dow Chemical), 858 g of EUDRAGIT® L100-55, and 754 g of lactose were mixed in a granulator. Wet granulation was carried out by adding an aqueous solution of 52 g of hydroxypropyl cellulose dissolved in a suitable amount of purified water to this mixture. The resulting granules were heat-dried in a tray dryer, and sieved to obtain the desired granule size. After sieving, 1 g of magnesium stearate based on 99 g of granules was added and mixed, and a rotary tabletting machine was used to make tablets with 8 mm diameters containing 10 mg of donepezil hydrochloride in a 200 mg tablet. Opadry yellow (Colorcon Japan Limited) was used to give the resulting tablets a water-soluble coating containing hydroxypropyl methylcellulose as its main component (coating amount: 8 mg/tablet) to obtain film-coated tablets. The results of the dissolution test are shown in Table 4.

### EXAMPLE 17

130 g of donepezil hydrochloride (Eisai Co. Ltd.), 832 g of ETHOCEL® 10FP (ethylcellulose, Dow Chemical), 962 g of EUDRAGIT® L100-55, and 598 g of lactose were mixed in a granulator. Wet granulation was carried out by adding an aqueous solution of 52 g of hydroxypropyl cellulose dissolved in a suitable amount of purified water to the mixture, and the resulting granules were heat-dried using a tray drier, and sieved to obtain the desired granule size. After sieving, 1 g of magnesium stearate based on 99 g of granules was added and mixed, and a rotary tabletting machine was used to form tablets with 8 mm diameters containing 10 mg of donepezil hydrochloride in a 200 mg tablet. Using Opadry Yellow (Colorcon Japan Limited), these tablets were then given a water-soluble film coating (coating amount: 8 mg/tablet) containing hydroxypropyl methylcellulose as its main component to obtain film-coated tablets. The results of the dissolution test are shown in Table 4.

### EXAMPLE 18

12 g of memantine hydrochloride (Lachema s.r.o., Czech Republic), 28.8 g of ETHOCEL® 10FP (ethylcellulose, Dow Chemical), 36 g of EUDRAGIT® L100-55, and 39.6 g of lactose were mixed in a granulator. Wet granulation was carried out by adding an aqueous solution of 2.4 g of hydroxypropyl cellulose dissolved in a suitable amount of purified water to the mixture, and the resulting granules were heat-dried using a tray drier, and sieved to obtain the desired granule size. After sieving, 1 g of magnesium stearate based on 99 g of granules was added and mixed, and a rotary tabletting machine was used to form tablets with 8 mm diameters containing 10 mg of memantine hydrochloride in a 200 mg tablet.

### EXAMPLE 19

6 g of donepezil hydrochloride (Eisai Co. Ltd.), 12 g of memantine hydrochloride (Lachema s.r.o.), 28.8 g of ETHOCEL® 10FP (ethylcellulose, Dow Chemical), 36 g of EUDRAGIT® L100-55, and 45.6 g of lactose were mixed in a granulator. Wet granulation was carried out by adding an aqueous solution of 2.4 g of hydroxypropyl cellulose dissolved in a suitable amount of purified water to the mixture, and the resulting granules were heat-dried in a tray drier, and sieved to obtain the desired granule size. After sieving, 1 g of magnesium stearate based on 109 g of granules was added and mixed, and a rotary tabletting machine was used for tabletting, resulting in a compression molded product with an 8 mm diameter containing 10 mg of donepezil hydrochloride and 20 mg of memantine hydrochloride in a 220 mg tablet. Opadry yellow (Colorcon Japan Limited) was used to give this compression molded product a water-soluble film coating containing hydroxypropyl methylcellulose as its main component (coating amount: 8 mg/tablet), resulting in film-coated tablets.

### COMPARATIVE EXAMPLE 1

300 mg of donepezil hydrochloride (Eisai Co. Ltd.), 750 mg of ETHOCEL® 10FP (ethylcellulose, Dow Chemical), 1920 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 1.

### COMPARATIVE EXAMPLE 2

300 mg of donepezil hydrochloride (Eisai Co. Ltd.), 375 mg of ETHOCELO® 10FP (ethylcellulose, Dow Chemical), 2295 mg of lactose and 30 mg of magnesium stearate were mixed in a mortar. 200 mg of this mixture was taken and made into tablets using an Autograph AG5000A (Shimazu Corporation) to obtain tablets with 8 mm diameters containing 20 mg of donepezil hydrochloride. The results of the dissolution test are shown in Table 5.

### EXAMPLE 20

7 grams donepezil hydrochloride, 37.8 grams ETHOCEL® 10FP, 22.4 g EUDRAGIT® L100-55, and 68.18 g lactose were mixed in a granulator. Wet granulation was carried out by adding an aqueous solution of 4.2 g of hydroxypropyl cellulose dissolved in a suitable amount of purified water to the mixture, and the resulting granules were heat-dried in a tray drier, and sieved to obtain the desired granule size. After sieving, 0.3 grams magnesium stearate based on 99.7 grams of granules was added and mixed, and a single punch tabletting machine was used to form a tablet with 8 mm in diameter containing 10 mg donepezil hydrochloride in 200 mg of the tablet. The results of the dissolution test are shown in Table 4.

### EXAMPLES 21-23 and COMPARATIVE EXAMPLE 3

In accordance with component amounts in Table 11, each component was mixed in a mortar. 200 mg of this mixture was taken and made into a tablet using an Autograph AG5000A to obtain a tablet weighing 200 mg with an 8 mm diameter containing 20 mg donepezil hydrochloride. The results of the dissolution test are shown in Tables 6 and 7.

### EXAMPLE 24

3.5 grams donepezil hydrochloride, 37.8 grams ETHOCEL® 10FP, 22.4 grams EUDRAGIT® LI100-55, and 73.5 g lactose (Pharmatose 200M manufactured by DMV Corporation) were mixed in a granulator. Wet granulation was carried out by adding an aqueous solution of 2.8 grams hydroxypropyl cellulose dissolved in a suitable amount of purified water to the mixture, and the resulting granules were heat dried in a tray drier, and sieved to obtain the desired granules size by a power mill. After sizing, 500 mg calcium stearate based on 5000 mg of granules was added and mixed, and an Autograph AG5000A was used to make a compression molded product with 8 mm in diameter containing 5 mg donepezil hydrochloride in 202 mg of the product with a compression pressure of 1200 Kgf.

### EXAMPLE 25

700 g donepezil hydrochloride, 2700 g ETHOCEL® 10FP, 2100 grams EUDRAGIT® L100-55, and 4250 g lactose were mixed in a granulator. Wet granulation was carried out by adding an aqueous solution of 220 grams of hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams granules was added and mixed, and a rotary tabletting machine was used to form a tablet with 8 mm in diameter containing 14 mg donepezil hydrochloride in 200 mg of the tablet. Opadry purple was used to give the resulting tablet a water-soluble film coating containing hydroxypropyl methylcellulose as its main component (coating amount: 8 mg/tablet), resulting in a film-coated tablet.

### EXAMPLE 26

700 grams donepezil hydrochloride, 2700 grams ETHOCEL® 10FP, 1900 grams EUDRAGIT® L100-55, and 4450 grams lactose were mixed in a granulator. Wet granulation was carried out by adding an aqueous solution of 220 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 14 mg of donepezil hydrochloride in 200 mg of the tablet. Opadry purple was used to give the resulting tablet a water-soluble film coating containing hydroxypropyl methylcellulose as its main component (coating amount: 8 mg/tablet), resulting in a film-coating tablet.

### EXAMPLE 27

700 grams donepezil hydrochloride, 2700 grams ETHOCEL® 10FP, 1900 grams EUDRAGIT® L100-55, and 4420 grams lactose were mixed in a granulator. Wet granulation was carried out by adding an aqueous solution of 250 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 14 mg of donepezil hydrochloride in 200 mg of the tablet. Opadry purple was used to give the resulting tablet a water-soluble film coating containing hydroxypropyl methylcellulose as its main component (coating amount: 8 mg/tablet), resulting in a film-coating tablet. The results of dissolution tests are shown in Table 8 and FIG. 7.

### EXAMPLE 28

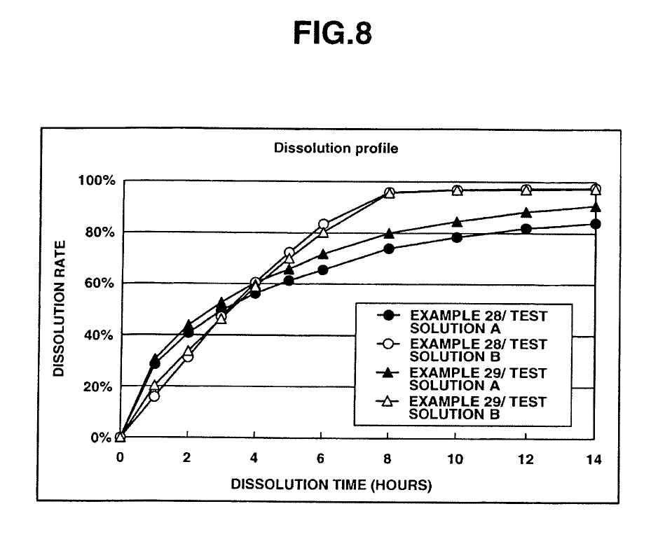

1050 grams donepezil hydrochloride, 3780 grams ETHOCEL® 10FP, 2240 grams EUDRAGIT® L100-55, and 6538 grams lactose were mixed in a granulator. Wet granulation was carried out by adding an aqueous solution of 350 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 15 mg of donepezil hydrochloride in 200 mg of the tablet. Opadry red was used to give the resulting tablet a water-soluble film coating containing hydroxypropyl methylcellulose as its main component (coating amount: 8 mg/tablet), resulting in a film-coating tablet. The results of dissolution tests are shown in Table 8 and FIG. 8.

### EXAMPLE 29

1400 grams donepezil hydrochloride, 3500 grams ETHOCEL® 10FP, 2520 grams EUDRAGIT® L100-55, and 6118 grams lactose were mixed in a granulator. Wet granulation was carried out by adding an aqueous solution of 420 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 20 mg of donepezil hydrochloride in 200 mg of the tablet. Opadry red was used to give the resulting tablet a water-soluble film coating containing hydroxypropyl methylcellulose as its main component (coating amount: 8 mg/tablet), resulting in a film-coating tablet. The results of dissolution tests are shown in Table 8 and FIG. 8.

### EXAMPLE 30

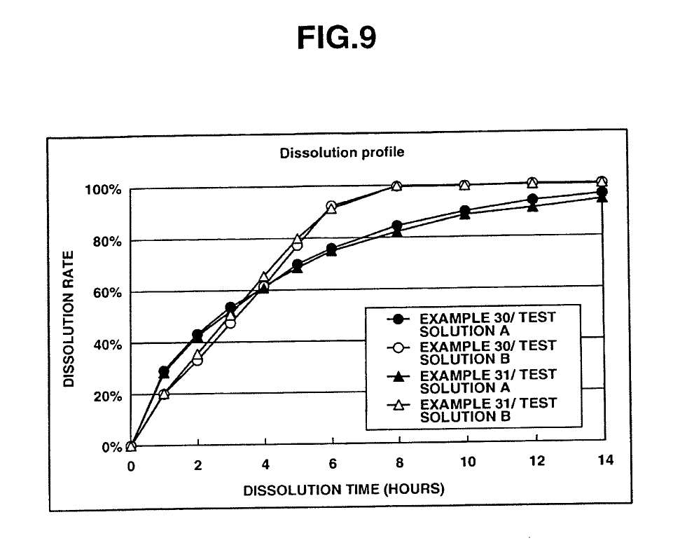

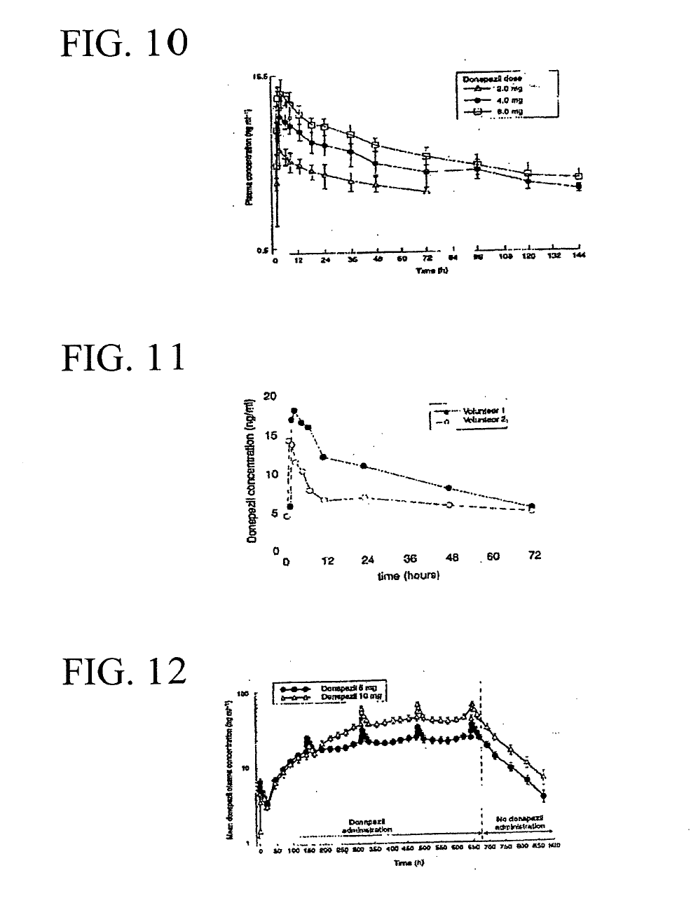

1150 grams donepezil hydrochloride, 2500 grams ETHOCEL® 10FP, 1800 grams EUDRAGIT® L100-55, and 4220 grams lactose were mixed in a granulator. Wet granulation was carried out by adding an aqueous solution of 300 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 23 mg of donepezil hydrochloride in 200 mg of the tablet. Opadry red was used to give the resulting tablet a water-soluble film coating containing hydroxypropyl methylcellulose as its main component (coating amount: 8 mg/tablet), resulting in a film-coating tablet. The results of dissolution tests are shown in Table 8 and FIG. 9.

### EXAMPLE 31

1150 grams donepezil hydrochloride, 2200 grams ETHOCEL® 10FP, 2100 grams EUDRAGIT® L100-55, and 4220 grams lactose were mixed in a granulator. Wet granulation was carried out by adding an aqueous solution of 300 grams hydroxypropyl cellulose dissolved in a suitable amount of purified water to the mixture, and the resulting granules were heat-dried in a fluidized bed drier, and sieved to obtain the desired granule size. After sizing, 0.3 grams magnesium stearate based on 99.7 grams of granules was added and mixed, and a rotary tabletting machine was used to form a tablet with 8 mm in diameter containing 23 mg of donepezil hydrochloride in 200 mg of the tablet. Opadry red was used to give the resulting tablet a water-soluble film coating containing hydroxypropyl methylcellulose as its main component (coating amount: 8 mg/tablet), resulting in a film-coating tablet. The results of dissolution tests are shown in Table 8 and FIG. 9.

### EXAMPLES 32 to 38

The film-coated tablets shown in Table 12 can be prepared according to the methods described herein. Table 12 shows amounts (milligrams) of each component in one film-coated table.

### EXAMPLE 39 to 44 and COMPARARIVE EXAMPLES 4 and 5

In accordance with component amounts in Table 13, each component was mixed in a mortar. 200 mg of this mixture was taken and made into tablets using an Autograph AG5000A to obtain a tablet (tablet weight: 200 mg) with 8 mm in diameter containing 20 mg memantine hydrochloride.

In yet embodiments, the invention provides other sustained-release formulations comprising at least one cholinesterase inhibitor in a matrix. The matrix may be any matrix that affords in vitro dissolution rates of the cholinesterase inhibitor within the ranges required and that releases the cholinesterase inhibitor in a pH independent manner. Preferably the matrix is a sustained release matrix, although normal release matrices having a coating that controls the release of the cholinesterase inhibitor may be used. In other embodiments of the invention, suitable materials for inclusion in the matrix of the sustained release formulations, in addition to one or more cholinesterase inhibitors are, for example:

(1) Hydrophilic polymers, such as gums (e.g., xanthan gum, locust bean gum), cellulose ethers (e.g., hydroxyalkylcelluloses and carboxyalkylcelluloses), acrylic resins and protein derived materials. The formulation may contain between 1% and 80% by weight of at least one hydrophilic polymer. The hydrophilic polymers can be any of those described in the application for any embodiment of the invention.

(2) Digestible, long chain (C₈-C₅₀, especially C₁₂ -C₄₀), substituted or unsubstituted hydrocarbons, such as fatty acids, fatty alcohols, glyceryl esters of fatty acids, mineral and vegetable oils and waxes. Hydrocarbons having a melting point of between 25° C. and 90° C. are preferred. Of these long chain hydrocarbon materials, fatty (aliphatic) alcohols are preferred. The formulation may contain up to 60% by weight of at least one digestible, long chain hydrocarbon.

(3) Polyalkylene glycols. The sustained release formulation may contain up to 60% by weight of at least one polyalkylene glycol.

One suitable matrix comprises at least one water soluble hydroxyalkyl cellulose, at least one C₁₂-C₃₆, preferably C₁₄ -C₂₂, aliphatic alcohol and, optionally, at least one polyalkylene glycol. The at least one hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the formulation will be determined by the rate of basic drug (e.g., cholinesterase inhibitor) release required. Preferably however, the sustained release formulation contains between 1% and 25%, especially between 5% and 15% by weight of the at least one hydroxyalkyl cellulose.

The at least one aliphatic alcohol may be, for example, lauryl alcohol, myristyl alcohol, stearyl alcohol or mixtures of two or more thereof. In other embodiments, the at least one aliphatic alcohol is cetyl alcohol, cetostearyl alcohol or a mixture thereof. The amount of the at least one aliphatic alcohol in the sustained release composition will be determined by the rate of basic drug (e.g., cholinesterase inhibitor) release required. It will also depend on whether at least one polyalkylene glycol is present in or absent from the formulation. In the absence of at least one polyalkylene glycol, the formulation preferably contains between 20% and 50% by weight of the at least one aliphatic alcohol. When at least one polyalkylene glycol is present in the formulation then the combined weight of the at least one aliphatic alcohol and the at least one polyalkylene glycol preferably constitutes between 20% and 50% by weight of the total formulation.

In one embodiment, the sustained release formulation comprises from 5 to 25% acrylic resin and from 8 to 40% by weight aliphatic alcohol by weight of the total formulation. A preferred acrylic resin comprises EUDRAGIT® RS PM, commercially available from Rohm Pharma.

In the formulation, the ratio of, e.g., the at least one hydroxyalkyl cellulose or acrylic resin to the at least one aliphatic alcohol/polyalkylene glycol determines, to a considerable extent, the release rate of the basic drug (e.g., cholinesterase inhibitor) from the formulation. A ratio of the at least one hydroxyalkyl cellulose to the at least one aliphatic alcohol/polyalkylene glycol of between 1:2 and 1:4 (or between 1:3 and 1:4) may be used.

The at least one polyalkylene glycol may be, for example, polypropylene glycol or polyethylene glycol. The number average molecular weight of the at least one polyalkylene glycol is preferred between 1000 and 15000 especially between 1500 and 12000. Another suitable sustained release matrix would comprise an alkylcellulose (especially ethyl cellulose), a C₁₂ to C₃₆ aliphatic alcohol and, optionally, a polyalkylene glycol.

In addition to the above ingredients, a sustained release matrix may also contain suitable quantities of other materials, e.g. diluents, lubricants, binders, granulating aids, colorants, flavorants and glidants that are conventional in the pharmaceutical art.

Matrix formulations can be prepared by methods known in the art, using compositions and materials known in the art, as described, for example, by Kydonieus, Controlled Release Technologies:Methods, Theory, and Applications, Volume II, pages 134-143, CRC Press, the disclosure of which is incorporated by reference herein in its entirety. For example, a drug and one or more of (i) a hydrophilic polymer, (ii) a digestible, long chain, substituted or unsubstituted hydrocarbon; and/or (iii) polyalkylene glycol can be used to form a matrix tablet. When the matrix tablet comes into contact with water, the outer layer forms a gel. The drug slowly diffuses through the gel layer over a period of time.

### EXAMPLE 45

Donepezil hydrochloride (150 grams), hydroxypropylmethylcellulose (HPMC) (650 grams, METHOCEL® K100M Premium, Dow Chemical Company), ethylcellulose (100 grams; Etocel 10FP, Dow Chemical Company), lactose (200 grams, Pharmatose 200M, DMV International) and citric acid (50 grams) will be blended and compressed with a roller compactor to form granules. Thereafter the compressed granules (1035 grams) will be blended with magnesium stearate (9 grams, Tyco International. Ltd.), and the resulting blend will be compressed into tablets with a tabletting machine.

EXAMPLE 45

| | |
|---|---|
| mg/tablet | Batch Scale (grams) |

Granules:

| | | |
|---|---|---|
| donepezil hydrochloride | 15 | 150 |
| hydroxypropylmethylcellulose | 65 | 650 |
| ethylcellulose (fine powder) | 10 | 100 |
| lactose | 20 | 200 |
| citric acid | 5 | 50 |
| Total: | 115 | 1150 |

Lubricant:

| | | |
|---|---|---|
| magnesium stearate | 1 | 9 |
| Total: | 116 | — |

As an alternative to or in addition to having a sustained release matrix, the matrix may be a normal release matrix having a coating that controls the release of the basic drug. In one embodiment, the formulation comprises film coated spheroids containing one or more basic drugs and a non-water soluble spheronizing agent. The term spheroid means a spherical granule having a diameter of between 0.5 mm and 2.5 mm especially between 0.5 mm and 2 mm. The spheronizing agent may be any pharmaceutically acceptable material that, together with the basic drug, can be spheronized to form spheroids. Microcrystalline cellulose is preferred. A suitable microcrystalline cellulose is, for example, the material sold as AVICEL® PH 101. The film coated spheroids can contain between 70% and 99% by weight, especially between 80% and 95% by weight, of the spheronizing agent, especially microcrystalline cellulose.

In addition to the basic drug and spheronizing agent, the spheroids may also contain a binder. Suitable binders, such as low viscosity, water soluble polymers, will be well known to those skilled in the pharmaceutical art. However, water soluble hydroxy C₁₋₆ alkyl cellulose, such as hydroxy propyl cellulose, are preferred. Additionally or alternatively, the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the basic drug at a sustained rate in an aqueous medium. The film coat is chosen so as to achieve, in combination with the other ingredients, the release rate described above. The film coat will generally include a water insoluble material such as (a) a wax, either alone or in admixture with a fatty alcohol, (b) shellac or zein, (c) a water insoluble cellulose, especially ethyl cellulose, (d) a polymethacrylate, especially EUDRAGIT®. Preferably, the film coat comprises a mixture of the water insoluble material and a water soluble material. The ratio of water insoluble to water soluble material is determined by, amongst other factors, the release rate required and the solubility characteristics of the materials selected. The water soluble material may be, for example, polyvinylpyrrolidone or, which is preferred, a water soluble cellulose, especially hydroxypropylmethyl cellulose. Suitable combinations of water insoluble and water soluble materials for the film coat include shellac and polyvinylpyrrolidone or, which is preferred, ethyl cellulose and hydroxypropylmethyl cellulose. Methods for preparing the sustained release formulations described in this embodiment of the invention are described, for example, in U.S. Pat. Nos. 5,656,295, 5,549,912, 5,508,042, 5,266,331 and 4,970,075 the disclosures of which are incorporated by reference herein in their entirety.

In another embodiment, the invention provides sustained release formulations that are membrane diffusion formulations (e.g., film coating(s) on a core; microencapsulation). In membrane diffusion formulations, a drug is released over time through one or more coatings which are each optionally composed of film-forming materials, plasticizers, pigments, and the like. In membrane diffusion formulations, the drug may be present within a core that has one or more coatings; on the surface of the core that has one or more coatings; or within one or more coatings that surround the core. Compositions of membrane diffusion formulations and methods for making them are described, for example, in WO 00/38686; WO 00/19985; U.S. Pat. No. 4,994,279; U.S. Pat. No. 4,894,239; Kydonieus, Controlled Release Technologies: Methods, Theory, and Applications, Volume II, pages 134-143, CRC Press; and Robinson, “Regulatory Guidelines for In-Vivo versus In-Vitro Correlations of Controlled Release Oral Products,” pages 73-87; the disclosures of each of which are incorporated by reference herein in their entirety.

Coating materials used to make membrane diffusion formulations are well known in the art. Exemplary coating materials used in membrane diffusion formulations include ammonio methacrylate copolymer Type B (EUDRAGIT® RS, Rohm); methacrylic acid copolymer Type B (EUDRAGIT® S, Rohm); ethylcellulose (ETOCEL®, Dow Chemical Company); an aqueous dispersion of ethylcellulose (AQUACOAT® ECD, FMC Biopolymer, which is a 30 percent by weight aqueous dispersion of ethylcellulose polymer); polyvinyl acetate; shellac; and combinations of two more thereof.

Any suitable material known in the art may be used as a core. Generally, the core must be pharmaceutically acceptable and have appropriate dimensions (e.g., 16-60 mesh) and firmness. Exemplary core materials include polymers (e.g., plastic resins); inorganic substances (e.g. silica, glass, hydroxyapatite, salts (e.g., sodium or potassium chloride, calcium or magnesium carbonate) and the like); organic substances (e.g., activated carbon), acids (e.g., citric, fumaric, tartaric, ascorbic and the like), and saccharides and derivatives thereof. In one embodiment, the core is a saccharide, such as sugars, oligosaccharides, polysaccharides and their derivatives. Exemplary saccharides suitable for use as a core material include glucose, rhamnose, galactose, lactose, sucrose, mannitol, sorbitol, dextrin, maltodextrin, cellulose, microcrystalline cellulose, sodium carboxymethyl cellulose, starches (e.g., maize, nice, potato, wheat, tapioca) and the like.

In one embodiment, the cores are 16-60 mesh sugar spheres (USP 22/NF XVII, page 1989) which comprise 62.5% to 91.5% (w/w) sucrose, where the remainder is starch and possibly dextrines, which are pharmaceutically inert or neutral. These cores are known in the art as neutral pellets. In one embodiment, nonpareil or microcrystalline cellulose are used as core materials. Exemplary nonpareil core materials include sucrose starch spheres (NP-101); purified sucrose spheres (NP-103), and lactose microcrystalline cellulose spheres (NP-105). There are three particle sizes for the sucrose starch spheres and the purified sucrose spheres: 20-24 Mesh type (850-710 μm); 24-32 Mesh type (710-500 μm); and 32-42 Mesh type (500-355 μm). Microcrystalline cellulose (CELPHERE™, Asahi Kasei) is available as grades: SCP-100 (75-212 μm), CP-203 (150-300 μm), CP-305 (300-500 μm), and CP-507 (500-710 μm).

### EXAMPLE 46

A suspension containing donepezil hydrochloride will be sprayed on nonpareil seeds using a centrifugal fluidized bed granulator. After coating the drug layer on the nonpareil seeds, a controlled release film will subsequently be coated on the drug layer using a fluid bed coater (e.g., Wurster type).

### EXAMPLE 47

Sustained release granules comprising 15 mg donepezil hydrochloride will be prepared. A drug suspension will be prepared as follows. Hydroxypropylcellulose (2.8 grams; HPC-L, Shin-Etsu Chemical Co., Ltd) will be dissolved in 192 ml ethanol. Light anhydrous silicic acid (5 grams; Aerosil 200, Degussa), talc (22 grams, NIPPON TALC Co., Ltd) and donepezil hydrochloride (60 grams; Eisai Co., Ltd) will be suspended in the HPC/ethanol solution whilst stirring. A sustained release film suspension will be prepared as follows. Talc (150 grams, NIPPON TALC Co., Ltd) and triethyl citrate will be suspended in ammonio methacrylate copolymer type B dispersion (500 grams; EUDRAGIT® RS 30D, Rohm). The suspension will then be further diluted with purified water (639.7 grams) with polyoxyethylene (20 grams) and sorbitan monooleate (0.3 grams; Tween 80, Nikko Chemicals).

A centrifugal fluidized-bed granulator equipped with a spray will be loaded with nonpareil seeds (270.2 grams, NP-101, Freund Corporation). The drug suspension will be sprayed on the nonpareil seeds to accumulate the drug layer on the nonpareil seeds by slow degrees in the granulator. The drug-coated nonpareil seeds will then be filled into a stainless steel drum and dried in a tray dryer at 40° C. The suspension for the sustained release film will be sprayed onto the drug-coated nonpareil seeds in a fluid bed coater. The nonpareil seeds will then be filled into a stainless steel drum as granules.

EXAMPLE NO. 47

| | |
|---|---|
| mg/day | Batch Scale (grams) |

Core:

| | | |
|---|---|---|
| nonpareil seed | 67.55 | 270.2 |

Drug Suspension:

| | | |
|---|---|---|
| donepezil hydrochloride | 15 | 60 |
| talc | 5.5 | 22 |
| Aerosil 200 | 1.25 | 5 |
| hydroxypropylcellulose | 0.7 | 2.8 |
| ethanol | — | 192 |

Sustained Release Film:

| | | |
|---|---|---|
| EUDRAGIT ® RS 30D | 5 | 500 |
| talc | 1.5 | 150 |
| triethyl citrate | 0.3 | 30 |
| Tween 80 | 0.003 | 0.3 |
| Purified water | — | 639.7 |
| Total | 96.083 | |

### EXAMPLE 48

Sustained release granules comprising 20 mg donepezil hydrochloride will be prepared. A drug suspension will be prepared as follows. Hydroxypropylcellulose (42 grams; HPC-L, Shin-Etsu Chemical Co., Ltd) will be dissolved in 2800 ml ethanol. Light anhydrous silicic acid (70 grams; Aerosil 200, Degussa), talc (280 grams, NIPPON TALC Co., Ltd) and donepezil hydrochloride (700 grams; Eisai Co., Ltd) will be suspended in the HPC/ethanol solution whilst stirring.

A sustained release film suspension will be prepared as follows. Hydroxypropylcellulose (31.5 grams; HPC-L, Shin-Etsu Chemical Co., Ltd) and shellac (140 grams, Japan Shellac Industries, Ltd.) will be dissolved in 800 ml ethanol. Talc (140 grams, NIPPON TALC Co., Ltd.), hydrogenated oil (595 grams; Lubriwax101, Freund Corporation) and ethylcellulose (26.25 grams, Etocel 10, Dow Chemical Company) will be suspended in the ethanol solution.

A centrifugal fluidized-bed granulator equipped with a spray will be loaded with nonpareil seeds (2870 grams, NP-103, Freund Corporation). The drug suspension will be sprayed on the nonpareil seeds to accumulate the drug layer by slow degrees in the granulator. The drug-coated seeds will then be filled into a stainless steel drum and dried in a tray dryer at 40° C.

The suspension for the sustained release film will be sprayed on the drug-coated nonpareil seeds in a fluid bed coater. The nonpareil seeds will then be filled into a stainless steel drum as granules.

EXAMPLE 48

| | |
|---|---|
| mg/day | Batch Scale (grams) |

Core:

| | | |
|---|---|---|
| nonpareil seed | 82 | 2870 |

Drug Suspension:

| | | |
|---|---|---|
| donepezil hydrochloride | 20 | 700 |
| talc | 8 | 280 |
| Aerosil 200 | 2 | 70 |
| hydroxypropylcellulose | 1.2 | 42 |
| ethanol | — | 2800 |

Sustained Release Film:

| | | |
|---|---|---|
| ethylcellulose | 0.75 | 26.25 |
| hydroxypropylcellulose | 0.9 | 31.5 |
| hydrogenated oil | 17 | 595 |
| shellac | 0.75 | 26.25 |
| talc | 4 | 140 |
| ethanol | — | 800 |
| Total | 136.6 | |

Microencapsulation technology includes (1) chemical methods, (2) physicochemical methods, and (3) physical methods. Chemical methods include interfacial polymerization methods; in-situ polymerization methods; and orifice methods (e.g., solidifying in liquid method; dripping method). Physicochemical methods include phase separation methods; solvent evaporation methods (e.g., drying in liquid method); and methods involving cooling melted dispersions. Physical methods include the Wurster method (e.g., fluidized bed technology); and spray-drying methods.

The phase separation method is one of the more common approaches to preparing sustained release formulations because it is a simple, well-known process and described, for example, by Lee and Robinson, “Methods to Achieve Sustained Drug Delivery,” Sustained and Controlled Release Drug Delivery Systems, pages 161-166, the disclosure of which is incorporated by reference in its entirety.

The solvent evaporation method includes the process of preparing a water-oil-water (w/o/w) emulsion or an oil-water-oil (o/w/o) emulsion. Microencapsulation by a water-oil-water emulsion method is described below. An aqueous solution (W1: internal water phase) is emulsified in an organic solvent having dissolved therein a hydrophobic polymer (oil phase) to obtain a water in oil emulsion. Thereafter, the water in oil emulsion is emulsified in a second aqueous solution comprising a protecting colloid (W2: outer water phase).

To cure the hydrophobic polymer, the solvent in the oil phase is evaporated from the water-oil-water emulsion by heating, pressure reduction, solvent extraction, cooling, lyophilization, or other methods known in the art.

Appropriate capsule materials and their corresponding solvents are listed in the table below.

| | Good Solvent for Use with |
|---|---|
| Capsule Material | Capsule Material |
| lactic acid/glycolic acid copolymer | methylene chloride |
| ethylcellulose | cyclohexane |
| polyvinyl acetate | chloroform |
| cellulose acetate phthalate | chloroform |
| dimethylaminoethylmethacrylate/ | chloroform/diethyl ether |
| methylmethacrylate copolymer | |

### EXAMPLE 49

A mixture of hydroxypropylmethylcellulose phthalate (800 grams; HPMCP, Shin-Etsu Chemical Co., Ltd) and donepezil hydrochloride (200 grams; Eisai Co., Ltd) will be granulated in a 30% ethanol solution (500 ml). After drying the wet granules, the core granules will be obtained with a No. 42 mesh screen.

A polydimethylsiloxane/silicone dioxide mixture (210 grams, Shin-Etsu Chemical Co Ltd), ethylcellulose (175 grams; ETOCEL® 10, Dow Chemical Company) and the core granules (750 grmas) will be suspended in 7000 ml cyclohexane at 80±5° C. Upon rapid cooling to room temperature (25° C.) with stirring, microcapsules will be produced in the suspension. The suspension will be stirred at a temperature of 5±3° C. The resulting microcapsules will be separated by filtration, and washed with hexane. After drying, the microcapsules will be selected to pass through a No. 30 mesh screen and a No. 140 mesh screen.

Thereafter, the microcapsules (681 grams) will be mixed with lactose (660 grams; Pharmatose, DMV International) and crospovidone (120 grams; BASF), and the dry mass will be passed through a roller compaction machine to make granules. The granules will be mixed with magnesium stearate (9 grams; Tyco International Ltd.) with a blender and will be compressed into tablets using a tableting machine.

EXAMPLE 49

| | | | | Batch Scale | |
|---|---|---|---|---|---|
| | | | mg/tablet | (grams) | |
| microcapsule | core | donepezil | 15 | 200 | |
| | | hydrochloride | | | |
| | | HPMCP | 60 | 800 | |
| | | 30% ethanolic solution | q.s. | 500 | |
| | capsule | polydimethylsiloxande | 21 | 210 | |
| | | and silicon | | | |
| | | dioxide mixture | | | |
| | | ethylcellulose | 17.5 | 175 | |
| | | cyclohexane | q.s. | 7000 | ml |

| | | |
|---|---|---|
| Total | 113.5 | — |

| | | | |
|---|---|---|---|
| diluent | lactose | 110 | 660 |
| disintegrator | crospovidone | 20 | 120 |
| lubricant | magnesium stearate | 1.5 | 9 |

| | | |
|---|---|---|
| Total | 245 | — |

### EXAMPLE 50

The oil phase will be prepared by dissolving a lactic acid/glycolic acid copolymer (1:1) (120 grams; PLGA-5015; Wako Pure Chemical Industries, Ltd.) in 140 ml methylene dichloride. The first water phase (W1) will be prepared by dissolving donepezil hydrochloride (1.5 grams; Eisai Co., Ltd) and D-mannitol (1 gram; Towa Chemical Industry Co., Ltd) in 27.5 ml purified water. The water phase will be added to the oil phase, and the water phase will be emulsified in the oil phase with stirring by the homogenizer (POLYTRON® dispersing machines, KINEMATICA) to produce a water-in-oil emulsion (i.e., W1/O emulsion).

The second water phase (W2) will be prepared by dissolving polyvinyl alcohol (150 grams; PVA, Kuraray Co., Ltd.) in 30 L purified water to produce a 0.5% polyvinyl alcohol solution for a protecting colloid. The water-in-oil emulsion (i.e., W1/O emulsion) and the 0.5% polyvinyl alcohol solution will be mixed with stirring to produce a water-oil-water emulsion.

The water-oil-water emulsion will be stirred for 5 hours using a propeller-type mixer to evaporate the methylene chloride. The microcapsules will be separated by filtration and repeatedly washed with purified water. The microcapsules will be dried for 20 hours at 25° C. with a vacuum drying machine. Thereafter, the microcapsules will be mixed with magnesium stearate (0.3 grams; Tyco International. Ltd.) to produce a powder comprising microcapsules.

EXAMPLE 50
(powder)

| | | | | Batch Scale | |
|---|---|---|---|---|---|
| | | | mg/day | (grams) | |
| microcapsule | W1 Phase | donepezil | 15 | 1.5 | |
| | | hydrochloride | | | |
| | | D-mannitol | 10 | 1 | |
| | | purified water | — | 27.5 | |
| | Oil Phase | copolymer of lactic | 1200 | 120 | |
| | | acid and glycolic acid | | | |
| | | (1:1) | | | |
| | | methylene dichloride | — | 140 | ml |
| | W2 Phase | PVA | 1500 | 150 | |
| | | purified water | — | 30 | L |

| | | | |
|---|---|---|---|
| Lubricant | magnesium stearate | 3 | 0.3 |

In other embodiments of the invention, a drug (e.g., cholinesterase inhibitor) and wax materials can be used to form sustained release formulations. Exemplary wax materials include sugar esters of fatty acids; glycerin esters of fatty acids; hydrogenated oils; and long chain alkyl alcohols.

In one embodiment, the invention provides a multi-layered granule comprising an inner, slow-releasing layer, an outer, rapid-releasing layer and an intermediate layer, provided between the slow-releasing layer and the rapid-releasing layer, which intermediate layer comprises a hardened oil and hydroxypropylcellulose and/or methylcellulose.

The granules of the invention comprise (i) a core, (ii) an inner, slow-releasing layer comprising a pharmacologically effective ingredient, (iii) an outer, rapid-releasing layer comprising a pharmacologically effective ingredient and (iv) an intermediate layer between the slow-releasing layer and the rapid-releasing layer, which intermediate layer comprises a hardened oil and hydroxypropylcellulose or methylcellulose.

It is preferable that the intermediate layer comprises 20 to 90 percent by weight of a hardened oil, 1 to 10 percent by weight of hydroxypropylcellulose and/or methylcellulose and the balance comprising a third component listed below. Examples of hardened oils suitable for use in the invention include hardened castor oil, rape oil, soybean oil, or a mixture of two or more thereof. The membranous intermediate layer between the slow and rapid release layers may contain hardened oil within the range of 20 to 90% by weight, or 20 to 80% by weight, based on the total weight of the membranous intermediate layer. A preferred content of hydroxypropylcellulose and/or methylcellulose suitable for use in the invention is between 1 to 10% by weight based on the total weight of the membranous layer.

Multi-layer granules can be produced with slow release granules as a starting material or with a granular seed. Suitable granular seeds (referred to as NPS) include generally-available granules formed of white sugar or a white sugar/corn starch mixture. The starting granule is, for instance, a pellet made by a process comprising kneading a mixture of drug to be slowly-released and other ingredients together with a binder, and extruding the resultant mixture. The invention is not limited to these processes. Seeds may be used in a conventional way to form a slow release layer surrounding it.

A membranous intermediate layer is formed over the slow release layer. The membranous intermediate layer comprises a hardened oil, hydroxypropylcellulose, methylcellulose, or a mixture of two or more thereof. The intermediate layer may be applied by spraying a liquid preparation onto the flowing and rolling materials that are to be coated. The liquid preparation is prepared in a procedure comprising mixing the ingredients with sucrose-fatty acid ester, talc, ethyl cellulose, or the like, and dissolving or dispersing the resulting mixture in a solvent such as ethyl alcohol. A rapid release layer is formed over the intermediate layer. This may be done using the same method that was used to form the intermediate layer. The thus-obtained multi-layer granular drugs may be used alone or in combination.

### EXAMPLE 51

Donepezil hydrochloride (150 grams; Eisai Co., Ltd.), sugar esters of fatty acids (600 grams; S-370, Mitsubishi-Kagaku Foods Corporation) and ethylcellulose (50 grams; ETCOCEL® 10, Dow Chemical Company) will be mixed with a high shear granulator; thereafter, ethanol and silicon oil (100 grams; Shin-Etsu Chemical Co., Ltd.) will be added and the mixture will be granulated. The granules will be oscillated in a granulator through a 0.5 mm screen, and the wet granules will be dried in a tray dryer at 40° C. After drying the wet granules, the sustained granules will be obtained by No. 30 sieve. The sustained release granules (810 grams) will be mixed with magnesium stearate (18 grams; Tyco International, Ltd.). The mixture will then be filled into HPMC capsules (Size No. 5; Shionogi Qualicaps Co., Ltd.) by 92 mg/capsule using an automatic capsule machine.

EXAMPLE 51

| | |
|---|---|
| mg/capsule | Batch Scale (grams) |

Granules:

| | | | |
|---|---|---|---|
| donepezil hydrochloride | 15 | 150 | |
| sugar esters of fatty acids (S-370) | 60 | 600 | |
| ethylcellulose | 5 | 50 | |
| silicon oil | 10 | 100 | |
| ethanol | q.s. | 100 | ml |

| | | |
|---|---|---|
| Total: | 90 | — |

Lubricant:

| | | |
|---|---|---|
| magnesium stearate | 2 | 18 |

Capsule:

| | | |
|---|---|---|
| HPMC Capsule No. 5 | — | — |

### EXAMPLE 52

Donepezil hydrochloride (150 grams; Eisai Co., Ltd.) and stearic monoglyceride (300 grams; MGS, Nikko Chemicals Co., Ltd) will be mixed with a high shear granulator. Octyldodecyl glyceride (50 grams, EXCEPARL® TGO, Kao Corporation) and ethanol (100 ml) will be slowly added into the mixer. Then, the resulting mixture will be kneaded for a few minutes, and will be granulated in a cylindrical granulator equipped with a screen having openings with a diameter of 0.5 mm. After drying the wet granules in a tray dryer, the sustained release granules will be obtained by putting them through a No. 30 sieve. The sustained release granules (400 grams) will then be mixed with lactose (8 grams; Pharmatose, DMV international) and crosscarmellose sodium (784 grams; Ac-Di-Sol, Asahi Kasei Corporation). A binder solution comprising Povidone (16 g; PVP-K30 BASF) will also be added by spraying. The resulting mixture will be granulated. The granules will be put through a screen in an oscillating granulator, and the wet granules will be dried in a tray dryer at 40° C. Thereafter, the granules and magnesium stearate (4 grams; Tyco International. Ltd.) will be mixed by a blender and compressed into tablets using a tableting machine.

EXAMPLE 52

| | | mg/tablet | Batch Scale (grams) | |
|---|---|---|---|---|
| Sustained | donepezil | 15 | 150 | |
| Release | hydrochloride | | | |
| Granule | stearic monoglyceride | 30 | 300 | |
| | octyldodecyl glyceride | 5 | 50 | |
| | Ethanol | q.s. | 100 | ml |
| | Total | 50 | — | |
| Disintegrating | crosscarmellose | 1 | 8 | |
| Agent | sodium | | | |
| Diluent | lactose | 98 | 784 | |
| Binder | Povidone | 2 | 16 | |
| Solvent | purified water | q.s. | — | |
| Lubricant | Magnesium stearate | 0.5 | 4 | |

| | | |
|---|---|---|
| Total: | 151.5 | — |

### EXAMPLE 53

Sustained release granules containing donepezil hydrochloride will be prepared in the same manner as Example 51.

Placebo granules will be prepared as follows. Hydroxypropylcellulose (100 grams) will be dissolved in purified water 400 ml. The HPC solution will be sprayed into D-mannitol (3950 grams; Towa Chemical Industry Co., Ltd.) and will be granulated. After drying the wet granules in a tray dryer, the placebo granules will be obtained by putting them through a sieve of 30 Mesh.

The sustained release granules comprising donepezil hydrochloride (810 grams) and the placebo granules (3645 grams) will be mixed with magnesium stearate (45 grams; Tyco International. Ltd.) to obtain mixed granules.

EXAMPLE 53

| | | | Batch |
|---|---|---|---|
| | | | Scale |
| | | mg/day | (grams) |
| Sustained Release | donepezil hydrochloride | 15 | 150 |
| Granule | sugar esters of fatty acids (S-370) | 60 | 600 |
| | ethylcellulose | 5 | 50 |
| | silicon oil | 10 | 100 |
| | ethanol | — | 200 |
| | Total: | 90 | — |
| Placebo Granule | D-mannitol | 395 | 3950 |
| | hydroxypropylcellulose | 10 | 100 |
| | purified water | — | 400 |
| | Total: | 405 | — |
| Lubricant | magnesium stearate | 5 | 45 |

| | | |
|---|---|---|
| Total | 500 | — |

In other embodiments, matrix diffusion formulations comprising one or more wax materials can be produced by melt granulation methods and can be used as the sustained release formulations of the invention.

Using melt granulation methods, spherical shaped particles can be obtained by spraying the melting wax in cooling air. The drug is suspended in the melting wax in advance. Exemplary waxes that are suitable for melt granulation methods include carnauba wax, hydrogenated oil, stearyl alcohol, glyceryl monostearate, paraffin, stearic acid, and the like.

### EXAMPLE 54

A sustained release granule will be prepared as follows. Donepezil hydrochloride (150 grams; Eisai Co., Ltd.) will be added to the molten mixture made by heating hydrogenated oil (2100 grams) and glyceryl monostearate (150 grams; MGS, Nikko Chemicals) at 85±3° C. CARBOPOL® 980 (260 g) and HPC-L (70 grams) will be suspended in the molten mixture while keeping the temperature at 85±3° C. The suspension will be sprayed in cool air to produce spherical granules. The granules will be passed through a No. 30 mesh screen and a No. 60 mesh screen. The granules (2184 grams) will be mixed with magnesium stearate (16 grams; Tyco International, Ltd.). Then the mixture will be filled into HPMC capsules (Size No. 2; Shionogi Qualicaps Co., Ltd.) at 275 mg/capsule using an automatic capsule machine.

| | | | Batch Scale |
|---|---|---|---|
| Example 54 (capsule) | | mg/capsule | (grams) |
| sustained | donepezil hydrochloride | 15 | 150 |
| release | hydrogenated oil | 210 | 2100 |
| granule | glyceryl monostearate | 15 | 150 |
| | Carbopol 980 | 26 | 260 |
| | HPC-L | 7 | 70 |
| | (Total) | 273 | — |
| lubricant | magnesium stearate | 2 | 16 |

| | | |
|---|---|---|
| total | 275 | — |

| | |
|---|---|
| Capsule | HPMC Capsule No. 2 |

### EXAMPLE 55

A sustained release granule will be prepared as follows. Donepezil hydrochloride (75 grams; Eisai Co., Ltd.) will be added to the molten mixture made by heating hydrogenated oil (3500 grams) and glyceryl monostearate (925 grams) at 93±3° C. CARBOPOL® 980 (450 grams) and xanthan gum (45 grams; Keltrol, CP Kelco, Inc) will be suspended in the molten mixture while keeping the temperature at 93±3° C. The suspension will be sprayed in cool air to produce spherical granules. The granules will be selected to pass through a No. 30 mesh screen and a No. 60 mesh screen. The granules (2997 grams) will be mixed with magnesium stearate (3 grams; Tyco International, Ltd.) to obtain the resulting product in the form of granules.

| | | | Batch Scale |
|---|---|---|---|
| Example 55 (granule) | | mg/capsule | (grams) |
| sustained | donepezil hydrochloride | 15 | 75 |
| release | carnauba wax | 700 | 3500 |
| granule | glyceryl monostearate | 185 | 925 |
| | Carbopol 980 | 90 | 450 |
| | xanthan gum | 9 | 45 |
| | (Total) | 999 | — |
| lubricant | magnesium stearate | 1 | 3 |

| | | |
|---|---|---|
| total | 1000 | — |

Another embodiment of the invention for sustained release formulations uses a drug carrier, wherein the drug is adsorbed on the surface of porous particle. Exemplary porous particles include calcium silicate (FLORITE®, Tokuyama Corporation), light anhydrous silicic acid (AEROSIL®, Degussa AG), synthetic aluminum silicate, silicon dioxide, magnesium aluminometasilicate, and the like.

### EXAMPLE 56

Donepezil hydrochloride (45 grams; Eisai Co., Ltd.) and succinic acid (15 grams) will be dissolved in 50% ethanol (2000 ml) comprising purified water. The solution will be added drop wise to calcium silicate (894 grams) and they will be sufficiently mixed. The mixture will be evaporated to dryness under reduced pressure to produce drug carriers. A molten mixture will be prepared by heating hydrogenated oil (1224 grams) and polyethylene glycol 6000 (136 grams, PEG6000, Sanyo Chemical Industries, Ltd.) to 90±3° C., and the molten mixture will be added to the drug carriers (636 grams) to produce granules. After cooling, the granules will be mixed with magnesium stearate (4 grams; Tyco International, Ltd.) to produce the resulting product in the form of granules (1000 mg/day).

| | | | | Batch Scale | |
|---|---|---|---|---|---|
| Example 56 (granule) | | | mg | (grams) | |
| sustained | drug carrier | donepezil hydrochloride | 15 | 45 | |
| release | | succinic acid | 5 | 15 | |
| granule | | calcium silicate | 298 | 894 | |
| | | 50% ethanol | q.s. | 2000 | ml |
| | | (total) | 318 | — | |
| | oil matrix | hydrogenated oil | 612 | 1224 | |
| | | PEG 6000 | 68 | 136 | |
| | | (total) | 998 | — | |
| Lubricant | | magnesium stearate | 2 | 4 | |

| | | |
|---|---|---|
| Total | 1000 | — |

### EXAMPLE 57

Donepezil hydrochloride (150 grams; Eisai Co., Ltd.) and citric acid (50 grams) will be dissolved in 50% ethanol (5000 ml) comprising purified water. The solution will be added drop wise to calcium silicate (2800 grams) and they will be sufficiently mixed. The mixture will be evaporated to dryness under reduced pressure to produce the drug carriers.

CARBOPOL® 980 (180 grams) and HPC-L (10 grams) will be suspended in a molten mixture comprising hydrogenated oil (1500 grams) and MGS-B (50 grams) at 90±3° C. The oil-based suspension will be added to the drug carriers (1500 grams). They will be fully mixed while gradually cooling to room temperature. The granules (2592 grams) will be mixed with magnesium stearate (8 grams; Tyco International, Ltd) to obtain the resulting product as granules (650 mg/day).

| | | | Batch Scale | |
|---|---|---|---|---|
| Example 57 (granule) | | mg | (grams) | |
| sustained release granule | donepezil hydrochloride | 15 | 150 | |
| | citric acid | 5 | 50 | |
| | silicon dioxide | 280 | 2800 | |
| | 50% ethanol | q.s. | 5000 | ml |
| | (total) | 300 | — | |
| | hydrogenated oil | 300 | 1500 | |
| | MGS | 10 | 50 | |
| | Carbopol 980 | 36 | 180 | |
| | HPC-L | 2 | 10 | |
| | (total) | 648 | — | |
| Lubricant | magnesium stearate | 2 | 8 | |

| | | |
|---|---|---|
| Total | 650 | — |

### EXAMPLE 58

Donepezil hydrochloride (450 grams; Eisai Co., Ltd.) and succinic acid (60 grams) will be dissolved in 50% ethanol (5000 ml) comprising purified water. The solution will be added drop wise to silicon dioxide (990 grams) and they will be sufficiently mixed. The mixture will be evaporated to dryness under reduced pressure to produce the drug carriers.

HPC-L (40 grams) will be suspended in a molten mixture comprising hydrogenated oil (3000 grams) and stearyl alcohol (60 grams; NOF CORPORATION) at a temperature of 90±3° C. The oil-based suspension will be added to the drug carriers (1000 grams). They will be fully mixed while gradually cooling to room temperature.

The granules (2050 grams) will be mixed with lactose (700 grams; Pharmatose, DMV International), Povidone (30 grams; Kollidon, BASF) and magnesium stearate (20 grams; Tyco International, Ltd.) in a blender. The mixture will be compressed into tablets using a tableting machine.

| | | | Batch | |
|---|---|---|---|---|
| Example 58 (tablet) | | mg | Scale (grams) | |
| Drug Carrier | donepezil hydrochloride | 15 | 450 | |
| | succinic acid | 2 | 60 | |
| | silicon dioxide | 33 | 990 | |
| | 50% ethanol | q.s. | 5000 | ml |
| | (total) | 50 | — | |
| Wax Matrix | Drug Carrier | 50 | 1000 | |
| | hydrogenated oil | 150 | 3000 | |
| | stearyl alcohol | 3 | 60 | |
| | HPC-L | 2 | 40 | |
| | (total) | 205 | — | |
| Diluent | lactose | 70 | 700 | |
| Binder | Povidone | 3 | 30 | |
| Lubricant | magnesium stearate | 2 | 20 | |

| | | |
|---|---|---|
| Total | 280 | — |

In other embodiments, the membrane diffusion formulations can be combined with the matrix formulations to form sustained release formulations.

### EXAMPLE 59

The core of the sustained granules will be prepared as follows. Donepezil hydrochloride (160 grams; Eisai Co., Ltd.), hydrogenated oil (680 grams; LUBRIWAX® 101, Freund Corporation), light anhydrous silicic acid (160 grams;Aerosil 200, Degussa ), and polyethylene glycol 6000 (120 grams; PEG-6000, NOF Corporation) will be mixed with-a high shear granulator. Thereafter, a solution of anhydrous citric acid (40g) and hydroxypropylcellulose (160 grams; HPC-L, Shin-Etsu Chemical Co., Ltd) in purified water (220 ml) will be sprayed into the previously-prepared donepezil mixture in a fluidized bed granulator. The wet granules will be dried in the fluidized bed dryer at 60° C. The core of the sustained release granules will be obtained by putting the granules through a sieve of 30 Mesh. The core will be a matrix diffusion formulation that functions as a sustained release formulation.

A coating for the cores will be prepared. Triethyl citrate (35 grams), talc (150 grams, Nippon Talc Co., Ltd.) and hydroxypropylcellulose (HPC-L; 60 grams) will be mixed in AQUACOAT® ECD (720 grams; FMC Biopolymer) to form a dispersion. AQUACOAT® ECD is a 30 percent by weight aqueous dispersion of ethylcellulose polymer. The dispersion will be sprayed onto the cores to produce sustained release granules. After drying the coated granules will be put through both sieves of 30 Mesh and 150 Mesh.

Thereafter, the coated granules (1281 grams) and magnesium stearate (21 grams; Tyco International. Ltd.) will be mixed together, and filled into HPMC capsules (Size No. 3; Shionogi Qualicaps Co., Ltd.) by 217 mg/capsule using standard automatic capsule filling machines.

| | | | | Batch Scale | |
|---|---|---|---|---|---|
| Example 59 (Capsule) | | | mg/day | (grams) | |
| Sustained | Core | donepezil hydrochloride | 20 | 160 | |
| Release | | hydrogenated oil | 85 | 680 | |
| Granule | | Aerosil 200 | 20 | 160 | |
| | | PEG 6000 | 15 | 120 | |
| | | hydroxypropylcellulose | 20 | 160 | |
| | | anhydrous citric acid | 5 | 40 | |
| | | purified water | — | 220 | ml |
| | | Total: | 165 | — | |
| | Controlled | AQUACOAT ® ECD | 72 (24) | 720 | |
| | Release | hydroxypropylcellulose | 6 | 60 | |
| | Coating | triethyl citrate | 3.5 | 35 | |
| | | talc | 15 | 150 | |

| | Total: | 48.5 | — |
|---|---|---|---|
| Lubricant | magnesium stearate | 3.5 | 6 |

| | | | |
|---|---|---|---|
| Capsule | HPMC Capsule No. 2 | — | — |

| | | |
|---|---|---|
| Total | 217 | — |

### EXAMPLE 60

Sustained release granules will be prepared in the same manner as Example 57.

Placebo granules will be prepared as follows. Hydroxypropylcellulose (100 grams) will be dissolved in purified water 300 ml. The HPC solution will be sprayed into D-mannitol (2735 grams; Towa Chemical Industry Co., Ltd.) and granulated. After drying the wet granules in a tray dryer, the placebo granules will be obtained by putting them through a sieve of 30 Mesh.

The sustained release granules comprising donepezil hydrochloride (1708 grams) and the placebo granules (2268 grams) will be mixed with magnesium stearate (24 grams; Tyco International. Ltd.) to obtain mixed granules.

| | | | | Batch Scale | |
|---|---|---|---|---|---|
| Example 60 (Granule) | | | mg/day | (grams) | |
| Sustained | Core | donepezil hydrochloride | 20 | 160 | |
| Release | | hydrogenated oil | 85 | 680 | |
| Granule | | Aerosil 200 | 20 | 160 | |
| | | PEG 6000 | 15 | 120 | |
| | | hydroxypropylcellulose | 20 | 160 | |
| | | anhydrous citric acid | 5 | 40 | |
| | | purified water | — | 220 | ml |
| | | Total: | 165 | — | |
| | Controlled | AQUACOAT ® ECD | 72 (24) | 720 | |
| | Release | hydroxypropylcellulose | 6 | 60 | |
| | Coating | triethyl citrate | 3.5 | 35 | |
| | | talc | 15 | 150 | |

| | Total: | 48.5 | — |
|---|---|---|---|
| Placebo Granule | D-mannitol | 273.5 | 2735 |
| | hydroxypropylcellulose | 10 | 100 |
| | purified water | — | 300 |
| | Total: | 283.5 | — |
| Lubricant | magnesium stearate | 3 | 24 |

| | | |
|---|---|---|
| Total | 500 | — |

In other embodiments, the sustained release formulations of the invention can be made using ion-exchange resin complexes. Ion-exchange resin complexes and methods for preparing them are known in the art and described for example, in U.S. Pat. No. 4,894,239 and by Lee and Robinson, “Methods to Achieve Sustained Drug Delivery,” Sustained and Controlled Release Drug Delivery Systems, pages 170-171, the disclosures of which are incorporated by reference in their entirety. Ion exchange resin materials are commercially available as Dowex® from Dow Chemical Company.

### EXAMPLE 61

A sustained release formulation comprising donepezil hydrochloride in the form of an ion-exchange resin complex will be prepared. Donepezil hydrochloride (60 grams; Eisai Co., Ltd.) and a positive ion exchange resin (80 grams) will be mixed well, with the addition of purified water, with a high shear mixer. The wet mass will be dried in a fluidized bed dryer at 60° C. to form a dried complex. The dried complex (140 grams), lactose (184 grams; Pharmatose, DMV International), hydroxypropylcellulose (HPC-L; 12 grams), microcrystalline cellulose (60 grams; AVICEL® PH103; Asahi Kasei), low-substituted hydroxypropylcellulose (40 grams; L-HPC, Shin-Etsu Chemical Co., Ltd) and magnesium stearate (1 gram; Tyco International, Ltd.) will be mixed in a blender and compressed into granules with a roller compactor. The granules and magnesium stearate (1 gram) will be mixed with a blender and compressed into tablets.

| | | | Batch | |
|---|---|---|---|---|
| Example 61 (capsule) | | mg/day | Scale (grams) | |
| Ion Exchange | donepezil hydrochloride | 15 | 60 | |
| Complex | positive ion exchange resin | 20 | 80 | |
| | purified water | q.s. | 500 | ml |
| | Total: | 35 | — | |
| Granule | lactose | 46 | 184 | |
| | hydroxypropylcellulose | 3 | 12 | |
| | microcrystalline cellulose | 15 | 60 | |
| | low-substituted | 10 | 40 | |
| | hydroxypropylcellulose | | | |
| | magnesium stearate | 0.25 | 1 | |

| | | |
|---|---|---|
| Total: | 74.25 | — |

| | | | |
|---|---|---|---|
| Lubricant | magnesium stearate | 0.25 | 1 |

| | | |
|---|---|---|
| Total | 109.5 | — |

In another example, a pulsed-release formulation may be used to achieve the objects of the invention. Pulsed-release formulations are designed to release the drug in pulses over a sustained period of time following administration to the patient. Pulsed-release formulations may combine an immediate-release formulation with a delayed-release formulation. A delayed-release formulation is achieved by releasing the drug after a pre-determined period of time. After that pre-determined period of time has elapsed, the release of the drug may be immediate, sustained or controlled. Pulsed-release formulations include, for example, multi-layered tablets (e.g., two or more layers); granules; and capsules comprising one or more immediate-release tablets and one or more delayed-release tablets.

### EXAMPLE 62

A sustained release granule will be prepared. Donepezil hydrochloride (1500 grams;

Eisai Co., Ltd), D-mannitol (5440 grams; Towa Chemical Industry Co., Ltd.) and crospovidone (2400 grams; Kollidon, BASF) will be mixed with a high shear granulator. Thereafter, a solution of hydroxypropylcellulose (300 grams; HPC-L, Shin-Etsu Chemical Co. Ltd.) in purified water (3600 ml) will be sprayed into the mixture in a fluidized bed granulator. The wet granules will be dried in the fluidized bed dryer at 60° C. The granules will be obtained by putting them through a sieve of 16 Mesh.

The granule (9640 grams) will be mixed with crospovidone (300 grams; Kollidon, BASF) and magnesium stearate (60 grams; Tyco International. Ltd) using a blender, and the resulting blend will be compressed into tablets using a tableting machine with a 4.8 mm diameter punch and die to prepare the core tablet.

Methacrylic acid copolymer, type A (1020 grams; EUDRAGIT® L-100, Rohm/Degussa), ethylcellulose (170 grams; Etocel, Dow Chemical Corporation) and triethyl citrate (220 grams) will be dissolved in ethanol. Talc (180 grams; Nippon Talc Co., Ltd.), titanium oxide (110 grams; Merck), and calcium stearate (700 grams; Taihei Chemical Industrial Co., Ltd.) will be suspended in the ethanol solution. The suspension will be sprayed onto the core tablets in a tablet coating machine (HICOARTER, Freund Corporation). Thereafter, the powder of carnauba wax will be added to the machine to provide a gloss over the tablet. The resulting table is a delayed-release tablet that will release the drug 8 hours after administration to the patient.

Both a core tablet (i.e., a tablet that does not have a coating) and a delayed-release tablet will be placed in a capsule to produce a sustained release capsule that will provide a pulse release of donepezil hydrochloride.

| Example 62 (tablet) | | mg | Batch Scale (grams) |
|---|---|---|---|
| Core Tablet | donepezil hydrochloride | 7.5 | 1500 |
| | D-mannitol | 27.2 | 5440 |
| | Crospovidone | 12 | 2400 |
| | HPC-L | 1.5 | 300 |
| | purified water | q.s. | 3600 |
| | Crospovidone | 1.5 | 300 |
| | magnesium stearate | 0.3 | 60 |
| | (total) | 50 | — |
| Coating | EUDRAGIT ® L-100 | 5.1 | 1020 |
| | ethylcellulose | 0.85 | 170 |
| | talc | 0.9 | 180 |
| | titanium oxide | 0.55 | 110 |
| | triethyl citrate | 1.1 | 220 |
| | calcium stearate | 3.5 | 700 |
| | ethanol | q.s. | 33000 |
| | carnauba wax | 0.001 | 0.2 |
| | (total) | 12 | — |
| Total | | 62 | — |

HPMC Capsule No. 2

The sustained release formulations of the invention can be used to treat or prevent various diseases characterized by symptoms of dementia and/or cognitive impairments. Such diseases include, for example, Alzheimer's disease (e.g., mild Alzheimer's disease, moderate Alzheimer's disease and/or severe Alzheimer's disease), Parkinson's disease, Huntington's disease, Pick's disease, vascular dementia, Lewy body dementia, and AIDS dementia.

The sustained release formulations of the invention can be used to treat cognitive impairments and/or dementia associated with neurologic and/or psychiatric conditions, including epilepsy, brain tumors, brain lesions, multiple sclerosis, Down's syndrome, Rett's syndrome, progressive supranuclear palsy, frontal lobe syndrome, and schizophrenia and related psychiatric disorders.

The sustained release formulations of the invention can be used to treat cognitive impairments caused by or associated with traumatic brain injuries (e.g., closed head injuries); post coronary artery by-pass surgery; electroconvulsive shock therapy; and chemotherapy.

The sustained release formulations of the invention can be used to treat or prevent attention deficit hyperactivity disorder (ADHD or ADD); prion diseases (e.g., Creutzfeldt-Jakob disease, variant Creutzfeldt-Jakob disease, Gerstmann-Straussler-Scheinker disease, familial insomnia); mild cognitive impairments; age-associated memory impairments; delirium; Tourette's syndrome; myasthenia gravis; autism; dyslexia; mania; depression; apathy; myopathy associated with or caused by diabetes; migraines (e.g., classic migraines, common migraines, complicated migraines, cluster headaches, menstrual and pre-menstrual migraines, abdominal migraines); and headaches (e.g., tension headaches).

The sustained release formulations of the invention can be used to delay the onset of Alzheimer's disease, to enhance cognitive functions, to treat and prevent sleep apnea and to alleviate withdrawal syndromes associated with tobacco, alcohol and/or drugs.

TABLE 1

| | | | |
|---|---|---|---|
| | TEST | TEST | TEST SOLUTION A/ |
| HOUR | SOLUTION A | SOLUTION B | TEST SOLUTION B |

COMPARATIVE EXAMPLE 1

| | | | |
|---|---|---|---|
| 1 h | 27% | 19% | 1.41 |
| 2 h | 41% | 27% | 1.50 |
| 3 h | 50% | 33% | 1.52 |
| 4 h | 57% | 37% | 1.54 |
| 5 h | 63% | 41% | 1.54 |
| 6 h | 67% | 44% | 1.54 |
| 8 h | 73% | 48% | 1.53 |

EXAMPLE 1

| | | | |
|---|---|---|---|
| 1 h | 22% | 15% | 1.52 |
| 2 h | 32% | 29% | 1.13 |
| 3 h | 39% | 40% | 0.97 |
| 4 h | 44% | 50% | 0.87 |
| 5 h | 47% | 58% | 0.81 |
| 6 h | 50% | 65% | 0.76 |
| 8 h | 55% | 78% | 0.71 |

TABLE 2

| | | | |
|---|---|---|---|
| | TEST | TEST | TEST SOLUTION A/ |
| HOUR | SOLUTION A | SOLUTION B | TEST SOLUTION B |

EXAMPLE 2

| | | | |
|---|---|---|---|
| 1 h | 18% | 14% | 1.28 |
| 2 h | 27% | 21% | 1.29 |
| 3 h | 33% | 26% | 1.27 |
| 4 h | 37% | 30% | 1.26 |
| 5 h | 41% | 33% | 1.25 |
| 6 h | 44% | 36% | 1.24 |
| 8 h | 50% | 41% | 1.23 |

EXAMPLE 3

| | | | |
|---|---|---|---|
| 1 h | 19% | 13% | 1.42 |
| 2 h | 25% | 19% | 1.36 |
| 3 h | 30% | 23% | 1.33 |
| 4 h | 34% | 26% | 1.31 |
| 5 h | 37% | 29% | 1.29 |
| 6 h | 40% | 31% | 1.28 |
| 8 h | 45% | 36% | 1.24 |

EXAMPLE 4

| | | | |
|---|---|---|---|
| 1 h | 8% | 10% | 0.75 |
| 2 h | 11% | 15% | 0.69 |
| 3 h | 12% | 19% | 0.64 |
| 4 h | 14% | 23% | 0.61 |
| 5 h | 15% | 25% | 0.59 |
| 6 h | 16% | 28% | 0.57 |
| 8 h | 17% | 31% | 0.54 |

EXAMPLE 5

| | | | |
|---|---|---|---|
| 1 h | 14% | 13% | 1.07 |
| 2 h | 20% | 20% | 0.96 |
| 3 h | 23% | 25% | 0.92 |
| 4 h | 26% | 29% | 0.88 |
| 5 h | 28% | 34% | 0.84 |
| 6 h | 30% | 41% | 0.74 |
| 8 h | 33% | 56% | 0.59 |

EXAMPLE 6

| | | | |
|---|---|---|---|
| 1 h | 17% | 15% | 1.15 |
| 2 h | 24% | 29% | 0.83 |
| 3 h | 29% | 41% | 0.70 |
| 4 h | 33% | 51% | 0.63 |
| 5 h | 35% | 60% | 0.59 |
| 6 h | 38% | 67% | 0.57 |
| 8 h | 42% | 77% | 0.55 |

TABLE 3

| | | | |
|---|---|---|---|
| | TEST | TEST | TEST SOLUTION A/ |
| HOUR | SOLUTION A | SOLUTION B | TEST SOLUTION B |

EXAMPLE 7

| | | | |
|---|---|---|---|
| 1 h | 15% | 16% | 0.98 |
| 2 h | 22% | 24% | 0.92 |
| 3 h | 26% | 30% | 0.86 |
| 4 h | 29% | 36% | 0.81 |
| 5 h | 31% | 45% | 0.68 |
| 6 h | 33% | 57% | 0.59 |
| 8 h | 36% | 69% | 0.53 |

EXAMPLE 8

| | | | |
|---|---|---|---|
| 1 h | 17% | 13% | 1.35 |
| 2 h | 25% | 26% | 0.98 |
| 3 h | 32% | 39% | 0.83 |
| 4 h | 37% | 51% | 0.73 |
| 5 h | 42% | 62% | 0.68 |
| 6 h | 45% | 71% | 0.63 |
| 8 h | 51% | 87% | 0.59 |

EXAMPLE 9

| | | | |
|---|---|---|---|
| 1 h | 16% | 15% | 1.06 |
| 2 h | 22% | 28% | 0.80 |
| 3 h | 26% | 39% | 0.68 |
| 4 h | 30% | 49% | 0.61 |
| 5 h | 33% | 57% | 0.57 |
| 6 h | 35% | 64% | 0.55 |
| 8 h | 39% | 76% | 0.51 |

EXAMPLE 10

| | | | |
|---|---|---|---|
| 1 h | 15% | 14% | 1.11 |
| 2 h | 22% | 27% | 0.82 |
| 3 h | 26% | 37% | 0.70 |
| 4 h | 30% | 46% | 0.64 |
| 5 h | 33% | 53% | 0.61 |
| 6 h | 35% | 60% | 0.58 |
| 8 h | 39% | 71% | 0.55 |

EXAMPLE 11

| | | | |
|---|---|---|---|
| 1 h | 25% | 16% | 1.56 |
| 2 h | 37% | 33% | 1.12 |
| 3 h | 46% | 52% | 0.88 |
| 4 h | 53% | 69% | 0.77 |
| 5 h | 59% | 83% | 0.71 |
| 6 h | 64% | 92% | 0.69 |
| 8 h | 70% | 99% | 0.71 |

TABLE 4

| | | | |
|---|---|---|---|
| | TEST | TEST | TEST SOLUTION A/ |
| HOUR | SOLUTION A | SOLUTION B | TEST SOLUTION B |

EXAMPLE 14

| | | | |
|---|---|---|---|
| 1 h | 36% | 32% | 1.13 |
| 2 h | 56% | 65% | 0.87 |
| 3 h | 69% | 87% | 0.79 |
| 4 h | 76% | 99% | 0.77 |
| 5 h | 81% | 101% | 0.80 |
| 6 h | 84% | 101% | 0.83 |

EXAMPLE 15

| | | | |
|---|---|---|---|
| 1 h | 21% | 13% | 1.60 |
| 2 h | 31% | 27% | 1.16 |
| 3 h | 40% | 47% | 0.85 |
| 5 h | 52% | 76% | 0.68 |
| 6 h | 57% | 86% | 0.66 |
| 8 h | 63% | 95% | 0.66 |
| 10 h | 68% | 97% | 0.71 |

EXAMPLE 16

| | | | |
|---|---|---|---|
| 1 h | 17% | 11% | 1.49 |
| 2 h | 25% | 21% | 1.17 |
| 3 h | 31% | 34% | 0.90 |
| 4 h | 35% | 50% | 0.72 |
| 5 h | 41% | 63% | 0.64 |
| 6 h | 45% | 75% | 0.60 |
| 8 h | 51% | 90% | 0.56 |
| 10 h | 56% | 97% | 0.58 |
| 12 h | 60% | 97% | 0.61 |
| 14 h | 63% | 97% | 0.65 |

EXAMPLE 17

| | | | |
|---|---|---|---|
| 1 h | 14% | 10% | 1.37 |
| 2 h | 21% | 16% | 1.27 |
| 3 h | 26% | 22% | 1.18 |
| 4 h | 30% | 28% | 1.08 |
| 5 h | 34% | 36% | 0.94 |
| 6 h | 37% | 47% | 0.79 |
| 8 h | 43% | 68% | 0.63 |
| 10 h | — | 82% | — |
| 12 h | — | 92% | — |
| 14 h | — | 98% | — |

EXAMPLE 20

| | | | |
|---|---|---|---|
| 1 h | 32.0 | 16.7 | 1.92 |
| 2 h | 43.0 | 27.0 | 1.59 |
| 3 h | 52.0 | 46.0 | 1.13 |
| 4 h | 58.0 | 65.3 | 0.89 |
| 5 h | 65.0 | 80.3 | 0.81 |
| 6 h | 72.0 | 92.3 | 0.78 |
| 8 h | 79.0 | 103.0 | 0.77 |
| 10 h | 84.0 | 104.0 | 0.81 |
| 12 h | 87.0 | 103.3 | 0.84 |
| 14 h | 90.0 | 103.7 | 0.87 |

TABLE 5

| | | | |
|---|---|---|---|
| | TEST | TEST | TEST SOLUTION A/ |
| HOUR | SOLUTION A | SOLUTION B | TEST SOLUTION B |

EXAMPLE 12

| | | | |
|---|---|---|---|
| 1 h | 12% | 22% | 0.55 |
| 2 h | 17% | 44% | 0.38 |
| 3 h | 20% | 61% | 0.32 |
| 4 h | 22% | 75% | 0.30 |
| 5 h | 25% | 85% | 0.29 |
| 6 h | 27% | 91% | 0.29 |
| 8 h | 30% | 94% | 0.32 |

EXAMPLE 13

| | | | |
|---|---|---|---|
| 1 h | 14% | 27% | 0.52 |
| 2 h | 19% | 50% | 0.38 |
| 3 h | 23% | 67% | 0.34 |
| 4 h | 26% | 78% | 0.34 |
| 5 h | 29% | 86% | 0.33 |
| 6 h | 31% | 91% | 0.34 |
| 8 h | 35% | 93% | 0.38 |

COMPARATIVE EXAMPLE 2

| | | | |
|---|---|---|---|
| 1 h | 71% | 53% | 1.34 |
| 2 h | 92% | 70% | 1.32 |
| 3 h | 96% | 76% | 1.26 |
| 4 h | 97% | 80% | 1.20 |
| 5 h | 97% | 83% | 1.16 |
| 6 h | 97% | 86% | 1.13 |
| 8 h | 97% | 88% | 1.09 |

TABLE 6

| | | | |
|---|---|---|---|
| | TEST | TEST | TEST SOLUTION A/ |
| HOUR | SOLUTION A | SOLUTION B | TEST SOLUTION B |

EXAMPLE 21

| | | | |
|---|---|---|---|
| 1 h | 12% | 8% | 1.50 |
| 2 h | 19% | 13% | 1.46 |
| 3 h | 25% | 19% | 1.32 |
| 4 h | 29% | 23% | 1.26 |
| 5 h | 32% | 26% | 1.23 |
| 6 h | 35% | 29% | 1.21 |
| 8 h | 39% | 34% | 1.15 |
| 10 h | 43% | 38% | 1.13 |
| 12 h | 46% | 41% | 1.12 |
| 14 h | 49% | 44% | 1.11 |

COMPARATIVE EXAMPLE 1

| | | | |
|---|---|---|---|
| 1 h | 27% | 19% | 1.41 |
| 2 h | 41% | 27% | 1.50 |
| 3 h | 50% | 33% | 1.52 |
| 4 h | 57% | 37% | 1.54 |
| 5 h | 63% | 41% | 1.54 |
| 6 h | 67% | 44% | 1.54 |
| 8 h | 73% | 48% | 1.53 |

TABLE 7

| | | | |
|---|---|---|---|
| | TEST | TEST | TEST SOLUTION A/ |
| HOUR | SOLUTION A | SOLUTION B | TEST SOLUTION B |

EXAMPLE 22

| | | | |
|---|---|---|---|
| 1 h | 11% | 12% | 0.92 |
| 2 h | 17% | 22% | 0.77 |
| 3 h | 21% | 30% | 0.70 |
| 4 h | 25% | 38% | 0.66 |
| 5 h | 28% | 46% | 0.61 |
| 6 h | 30% | 52% | 0.58 |
| 8 h | 34% | 64% | 0.53 |
| 10 h | 37% | 74% | 0.50 |
| 12 h | 40% | 82% | 0.49 |
| 14 h | 42% | 88% | 0.48 |

EXAMPLE 23

| | | | |
|---|---|---|---|
| 1 h | 10% | 21% | 0.48 |
| 2 h | 16% | 41% | 0.39 |
| 3 h | 19% | 56% | 0.34 |
| 4 h | 21% | 71% | 0.30 |
| 5 h | 23% | 82% | 0.28 |
| 6 h | 26% | 91% | 0.29 |
| 8 h | 29% | 105% | 0.28 |
| 10 h | 32% | 108% | 0.30 |
| 12 h | 35% | 109% | 0.32 |
| 14 h | 38% | 109% | 0.35 |

COMPARATIVE EXAMPLE 3

| | | | |
|---|---|---|---|
| 1 h | 68% | 88% | 0.77 |
| 2 h | 94% | 98% | 0.96 |
| 3 h | 97% | 101% | 0.96 |
| 4 h | 97% | 101% | 0.96 |
| 5 h | 97% | 102% | 0.95 |
| 6 h | 98% | 102% | 0.96 |
| 8 h | 97% | 102% | 0.95 |
| 10 h | 98% | 102% | 0.96 |
| 12 h | 98% | 102% | 0.96 |
| 14 h | 98% | 103% | 0.95 |

TABLE 8

| | | | |
|---|---|---|---|
| | TEST | TEST | TEST SOLUTION A/ |
| HOUR | SOLUTION A | SOLUTION B | TEST SOLUTION B |

EXAMPLE 27

| | | | |
|---|---|---|---|
| 1 h | 29% | 22% | 1.30 |
| 2 h | 42% | 35% | 1.20 |
| 3 h | 53% | 52% | 1.01 |
| 4 h | 61% | 71% | 0.86 |
| 5 h | 68% | 87% | 0.78 |
| 6 h | 73% | 97% | 0.75 |
| 8 h | 81% | 103% | 0.78 |
| 10 h | 86% | 104% | 0.83 |
| 12 h | 89% | 104% | 0.86 |
| 14 h | 92% | 104% | 0.88 |

EXAMPLE 28

| | | | |
|---|---|---|---|
| 1 h | 29% | 15% | 1.87 |
| 2 h | 41% | 31% | 1.32 |
| 3 h | 50% | 47% | 1.06 |
| 4 h | 56% | 61% | 0.93 |
| 5 h | 61% | 72% | 0.85 |
| 6 h | 66% | 84% | 0.78 |
| 8 h | 74% | 96% | 0.77 |
| 10 h | 79% | 97% | 0.81 |
| 12 h | 82% | 97% | 0.84 |
| 14 h | 84% | 98% | 0.86 |

EXAMPLE 29

| | | | |
|---|---|---|---|
| 1 h | 31% | 20% | 1.51 |
| 2 h | 44% | 34% | 1.29 |
| 3 h | 53% | 47% | 1.14 |
| 4 h | 60% | 59% | 1.02 |
| 5 h | 66% | 70% | 0.94 |
| 6 h | 72% | 81% | 0.89 |
| 8 h | 80% | 96% | 0.83 |
| 10 h | 85% | 97% | 0.87 |
| 12 h | 89% | 97% | 0.91 |
| 14 h | 91% | 97% | 0.94 |

EXAMPLE 30

| | | | |
|---|---|---|---|
| 1 h | 29% | 20% | 1.45 |
| 2 h | 43% | 33% | 1.30 |
| 3 h | 54% | 47% | 1.14 |
| 4 h | 62% | 61% | 1.01 |
| 5 h | 70% | 77% | 0.90 |
| 6 h | 76% | 92% | 0.82 |
| 8 h | 85% | 99% | 0.85 |
| 10 h | 90% | 99% | 0.90 |
| 12 h | 94% | 100% | 0.94 |
| 14 h | 96% | 100% | 0.96 |

EXAMPLE 31

| | | | |
|---|---|---|---|
| 1 h | 29% | 21% | 1.39 |
| 2 h | 43% | 36% | 1.20 |
| 3 h | 52% | 51% | 1.03 |
| 4 h | 61% | 66% | 0.93 |
| 5 h | 69% | 80% | 0.86 |
| 6 h | 75% | 91% | 0.82 |
| 8 h | 83% | 100% | 0.83 |
| 10 h | 88% | 100% | 0.88 |
| 12 h | 91% | 100% | 0.91 |
| 14 h | 94% | 100% | 0.94 |

TABLE 9

| | | | |
|---|---|---|---|
| | TEST | TEST | TEST SOLUTION A/ |
| HOUR | SOLUTION A | SOLUTION B | TEST SOLUTION B |

EXAMPLE 40

| | | | |
|---|---|---|---|
| 1 h | 10% | 12% | 0.91 |
| 2 h | 14% | 18% | 0.81 |
| 3 h | 18% | 22% | 0.80 |
| 4 h | 19% | 27% | 0.70 |
| 6 h | 22% | 34% | 0.64 |
| 8 h | 23% | 41% | 0.57 |
| 12 h | 28% | 49% | 0.57 |

EXAMPLE 41

| | | | |
|---|---|---|---|
| 1 h | 26% | 20% | 1.33 |
| 2 h | 39% | 30% | 1.32 |
| 3 h | 49% | 39% | 1.25 |
| 4 h | 57% | 46% | 1.23 |
| 6 h | 67% | 56% | 1.20 |
| 8 h | 77% | 63% | 1.22 |
| 12 h | 87% | 70% | 1.24 |

EXAMPLE 42

| | | | |
|---|---|---|---|
| 1 h | 10% | 21% | 0.49 |
| 2 h | 14% | 32% | 0.44 |
| 3 h | 18% | 39% | 0.46 |
| 4 h | 20% | 44% | 0.46 |
| 6 h | 25% | 59% | 0.42 |
| 8 h | 29% | 64% | 0.46 |
| 12 h | 35% | 68% | 0.51 |

COMPARATIVE EXAMPLE 4

| | | | |
|---|---|---|---|
| 1 h | 40% | 37% | 1.09 |
| 2 h | 57% | 51% | 1.12 |
| 3 h | 66% | 60% | 1.10 |
| 4 h | 74% | 69% | 1.08 |
| 6 h | 86% | 80% | 1.07 |
| 8 h | 91% | 84% | 1.08 |
| 12 h | 97% | 94% | 1.03 |

TABLE 10

| | | | |
|---|---|---|---|
| | TEST | TEST | TEST SOLUTION A/ |
| HOUR | SOLUTION A | SOLUTION B | TEST SOLUTION B |

EXAMPLE 43

| | | | |
|---|---|---|---|
| 1 h | 31% | 26% | 1.16 |
| 2 h | 46% | 44% | 1.04 |
| 3 h | 57% | 59% | 0.96 |
| 4 h | 64% | 68% | 0.94 |
| 6 h | 74% | 86% | 0.88 |
| 8 h | 83% | 90% | 0.92 |
| 12 h | 91% | 97% | 0.94 |

COMPARATIVE EXAMPLE 5

| | | | |
|---|---|---|---|
| 1 h | 67% | 87% | 0.77 |
| 2 h | 95% | 90% | 1.06 |
| 3 h | 96% | 94% | 1.02 |
| 4 h | 96% | 95% | 1.01 |
| 6 h | 97% | 94% | 1.03 |
| 8 h | 95% | 93% | 1.02 |
| 12 h | 94% | 93% | 1.01 |

TABLE 11

| | | | | | COMPARATIVE |
|---|---|---|---|---|---|
| NAME OF COMPONENT | VENDOR | EXAMPLE 21 | EXAMPLE 22 | EXAMPLE 23 | EXAMPLE 3 |
| Donepezil.HCl | Eisai | 300 | 300 | 300 | 300 |
| Ethocel 10 FP | Dow Chemical | 750 | — | — | — |
| Eudragit RS PO | Röhm | — | 750 | 750 | 750 |
| Eudragit L100-55 | Röhm | — | 1500 | — | — |
| Eudragit L100 | Röhm | 1500 | — | — | — |
| AQOAT LF | SHIN-ETSU CHEMICAL | — | — | 1500 | — |
| LACTOSE (FlowLac 100) | Meggle | 420 | 435 | 435 | 1935 |
| MAGNESIUM STEARATE | Mallinckrodt | 30 | 15 | 15 | 15 |

| | | | | |
|---|---|---|---|---|
| Total (mg) | 3000 | 3000 | 3000 | 3000 |

TABLE 12

| | | EXAMPLE | EXAMPLE | EXAMPLE | EXAMPLE | EXAMPLE | EXAMPLE | EXAMPLE |
|---|---|---|---|---|---|---|---|---|
| NAME OF COMPONENT | VENDOR | 32 | 33 | 34 | 35 | 36 | 37 | 38 |
| Donepezil.HCl | Eisai | 12 | 12 | 14 | 18 | 18 | 30 | 30 |
| Ethocel 10 FP | Dow Chemical | 54 | 54 | 48 | 54 | 44 | 50 | 44 |
| Eudragit L100-55 | Röhm | 32 | 38 | 44 | 38 | 42 | 36 | 42 |
| Lactose (Pharmatose 200M) | DMV International | 97.4 | 91.4 | 88.4 | 84.4 | 90.4 | 77.4 | 77.4 |
| HPC-L | Nippon Soda | 4 | 4 | 5 | 5 | 5 | 6 | 6 |
| Magnesium Stearate | Mallinckrodt | 0.6 | 0.6 | 0.6 | 0.6 | 0.6 | 0.6 | 0.6 |
| tablet total (mg) | | 200 | 200 | 200 | 200 | 200 | 200 | 200 |
| Opadry purple (mg/tablet) | Colorcon Japan | 8 | 8 | 8 | 8 | 8 | 8 | 8 |

| | | | | | | | |
|---|---|---|---|---|---|---|---|
| Film-coated total (mg) | 208 | 208 | 208 | 208 | 208 | 208 | 208 |

TABLE 13

| | | EXAM- | | EXAM- | | | | | |
|---|---|---|---|---|---|---|---|---|---|
| NAME OF | | PLE | EXAMPLE | PLE | EXAMPLE | EXAMPLE | EXAMPLE | COMPARATIVE | COMPARATIVE |
| COMPONENT | VENDOR | 39 | 40 | 41 | 42 | 43 | 44 | EXAMPLE 4 | EXAMPLE 5 |
| Memantine.HCl | Lachema s.r.o | 300 | 300 | 300 | 300 | 300 | 300 | 300 | 300 |
| Ethocel 10 FP | Dow Chemical | — | 750 | 750 | 750 | — | — | 750 | — |
| Eudragit RS PO | Röhm | — | — | — | — | 750 | 750 | — | 750 |
| Eudragit L100-55 | Röhm | 1500 | 1500 | — | — | 1500 | — | — | — |
| Eudragit L100 | Röhm | — | — | 1500 | — | — | — | — | — |
| AQOAT LF | SHIN-ETSU | — | — | — | 1500 | — | 1500 | — | — |
| | CHEMICAL | | | | | | | | |
| LACTOSE | Meggle | 1170 | 420 | 420 | 420 | 435 | 435 | 1920 | 1935 |
| (FlowLac 100) | | | | | | | | | |
| MAGNESIUM | Mallinckrodt | 30 | 30 | 30 | 30 | 15 | 15 | 30 | 15 |
| STEARATE | | | | | | | | | |

| | | | | | | | | |
|---|---|---|---|---|---|---|---|---|
| Total (mg) | 3000 | 3000 | 3000 | 3000 | 3000 | 3000 | 3000 | 3000 |

Each of the patents, patent applications, and publications cited herein are incorporated by reference herein in their entirety.

It will be apparent to one skilled in the art that various modifications can be made to the invention without departing from the spirit or scope of the appended claims.

What is claimed is:

1. An orally administrable formulation comprising from 1 milligram to 50 milligrams donepezil or a pharmaceutically acceptable salt thereof; wherein the formulation provides a blood plasma concentration level in a patient wherein the ratio of the maximum steady state plasma concentration to the minimum steady state plasma concentration is from 1.00 to 1.50; and wherein the steady state plasma concentration is measured after a patient is administered the formulation on a daily basis for at least three weeks.

2. The formulation of claim 1, comprising from 10 milligrams to 25 milligrams donepezil or a pharmaceutically acceptable salt thereof.

3. The formulation of claim 1, wherein the ratio of the maximum steady state plasma concentration to the minimum steady state plasma concentration is from 1.05 to 1.30.

4. An orally administrable formulation comprising from 10 milligrams to 25 milligrams donepezil or a pharmaceutically acceptable salt thereof; wherein the formulation provides a blood plasma concentration level in a patient wherein the maximum steady state plasma concentration is 5% to 15% higher than the average steady state plasma concentration; and wherein the steady state plasma concentration is measured after a patient is administered the formulation on a daily basis for at least three weeks.

5. The formulation of claim 4, wherein the maximum steady state plasma concentration is 5% to 12% higher than the average steady state plasma concentration.

6. An orally administrable formulation comprising from 10 milligrams to 25 milligrams donepezil or a pharmaceutically acceptable salt thereof; wherein the formulation provides a steady state plasma concentration in a patient from 2.0 to 3.0 ng/ml per milligram of donepezil or the pharmaceutically acceptable salt thereof in the formulation; and wherein the steady state plasma concentration is measured after a patient is administered the formulation on a daily basis for at least three weeks.

7. The formulation of claim 6, wherein the formulation provides a steady state plasma concentration from 28 to 42 ng/ml.

8. The formulation of claim 7, wherein the formulation comprises 14 milligrams donepezil or a pharmaceutically acceptable salt thereof.

9. The formulation of claim 6, wherein the formulation provides a steady state plasma concentration from 30 to 45 ng/ml.

10. The formulation of claim 9, wherein the formulation comprises 15 milligrams donepezil or a pharmaceutically acceptable salt thereof.

11. The formulation of claim 6, wherein the formulation provides a steady state plasma concentration from 40 to 60 ng/ml.

12. The formulation of claim 11, wherein the formulation comprises 20 milligrams donepezil or a pharmaceutically acceptable salt thereof.

13. The formulation of claim 6, wherein the formulation provides a steady state plasma concentration from 46 to 69 ng/ml.

14. The formulation of claim 13, wherein the formulation comprises 23 milligrams donepezil or a pharmaceutically acceptable salt thereof.

15. An orally administrable formulation comprising from 1 milligram to 50 milligrams donepezil or a pharmaceutically acceptable salt thereof; wherein the formulation provides a blood plasma concentration level in a patient wherein the maximum steady state plasma concentration is 1% to 40% higher than the minimum steady state plasma concentration; and wherein the steady state plasma concentration is measured after a patient is administered the formulation on a daily basis for at least three weeks.

16. The formulation of claim 15, wherein the maximum steady state plasma concentration is 5% to 30% higher than the minimum steady state plasma concentration.

17. The formulation of claim 16, wherein the maximum steady state plasma concentration is 5% to 20% higher than the minimum steady state plasma concentration.

18. An orally administrable formulation comprising from 1 milligram to 50 milligrams donepezil or a pharmaceutically acceptable salt thereof; wherein the formulation provides a blood plasma concentration level in a patient having a maximum steady state plasma concentration that is 5% to 25% higher than an average steady state plasma concentration; and wherein the steady state plasma concentration is measured after a patient is administered the formulation on a daily basis for at least three weeks.

19. The formulation of claim 18, wherein the maximum steady state plasma concentration is 5% to 23% higher than the average steady state plasma concentration.

20. The formulation of claim 18, wherein the maximum steady state plasma concentration is 5% to 20% higher than the average steady state plasma concentration.

21. The formulation of claim 18, wherein the maximum steady state plasma concentration is 5% to 18% higher than the average steady state plasma concentration.

22. The formulation of claim 18, comprising from 10 milligrams to 25 milligrams donepezil or a pharmaceutically acceptable salt thereof.

23. The formulation of claim 22, comprising 14 milligrams, 15 milligrams, 20 milligrams, or 23 milligrams donepezil or a pharmaceutically acceptable salt thereof.
